SEQUENCE LISTING (22 sequences)

SEQ ID NO: 1 - DNA, 19 bp, Artificial Sequence
  Description of Artificial Sequence Synthetic primer
ttggtcaccttcagcttgg

SEQ ID NO: 2 - DNA, 20 bp, Artificial Sequence
  Description of Artificial Sequence Synthetic oligonucleotide
ggcgagccgcgccgcgaagg

SEQ ID NO: 3 - DNA, 20 bp, Artificial Sequence
  Description of Artificial Sequence Synthetic oligonucleotide
gctcgccggggatccccgag

SEQ ID NO: 4 - DNA, 5420 bp, Artificial Sequence
  Description of Artificial Sequence Synthetic polynucleotide
atgagcaagctggagaagtttacaaactgctactccctgtctaagaccctgaggttcaaggccatccctgtgggcaagacccaggagaacatcgacaataagcggctgctggtggaggacgagaagagagccgaggattataagggcgtgaagaagctgctggatcgctactatctgtcttttatcaacgacgtgctgcacagcatcaagctgaagaatctgaacaattacatcagcctgttccggaagaaaaccagaaccgagaaggagaataaggagctggagaacctggagatcaatctgcggaaggagatcgccaaggccttcaagggcaacgagggctacaagtccctgtttaagaaggatatcatcgagacaatcctgccagagttcctggacgataaggacgagatcgccctggtgaacagcttcaatggctttaccacagccttcaccggcttctttgataacagagagaatatgttttccgaggaggccaagagcacatccatcgccttcaggtgtatcaacgagaatctgacccgctacatctctaatatggacatcttcgagaaggtggacgccatctttgataagcacgaggtgcaggagatcaaggagaagatcctgaacagcgactatgatgtggaggatttctttgagggcgagttctttaactttgtgctgacacaggagggcatcgacgtgtataacgccatcatcggcggcttcgtgaccgagagcggcgagaagatcaagggcctgaacgagtacatcaacctgtataatcagaaaaccaagcagaagctgcctaagtttaagccactgtataagcaggtgctgagcgatcgggagtctctgagcttctacggcgagggctatacatccgatgaggaggtgctggaggtgtttagaaacaccctgaacaagaacagcgagatcttcagctccatcaagaagctggagaagctgttcaagaattttgacgagtactctagcgccggcatctttgtgaagaacggccccgccatcagcacaatctccaaggatatcttcggcgagtggaacgtgatccgggacaagtggaatgccgagtatgacgatatccacctgaagaagaaggccgtggtgaccgagaagtacgaggacgatcggagaaagtccttcaagaagatcggctccttttctctggagcagctgcaggagtacgccgacgccgatctgtctgtggtggagaagctgaaggagatcatcatccagaaggtggatgagatctacaaggtgtatggctcctctgagaagctgttcgacgccgattttgtgctggagaagagcctgaagaagaacgacgccgtggtggccatcatgaaggacctgctggattctgtgaagagcttcgagaattacatcaaggccttctttggcgagggcaaggagacaaacagggacgagtccttctatggcgattttgtgctggcctacgacatcctgctgaaggtggaccacatctacgatgccatccgcaattatgtgacccagaagccctactctaaggataagttcaagctgtattttcagaaccctcagttcatgggcggctgggacaaggataaggagacagactatcgggccaccatcctgagatacggctccaagtactatctggccatcatggataagaagtacgccaagtgcctgcagaagatcgacaaggacgatgtgaacggcaattacgagaagatcaactataagctgctgcccggccctaataagatgctgccaaaggtgttcttttctaagaagtggatggcctactataaccccagcgaggacatccagaagatctacaagaatggcacattcaagaagggcgatatgtttaacctgaatgactgtcacaagctgatcgacttctttaaggatagcatctcccggtatccaaagtggtccaatgcctacgatttcaacttttctgagacagagaagtataaggacatcgccggcttttacagagaggtggaggagcagggctataaggtgagcttcgagtctgccagcaagaaggaggtggataagctggtggaggagggcaagctgtatatgttccagatctataacaaggacttttccgataagtctcacggcacacccaatctgcacaccatgtacttcaagctgctgtttgacgagaacaatcacggacagatcaggctgagcggaggagcagagctgttcatgaggcgcgcctccctgaagaaggaggagctggtggtgcacccagccaactcccctatcgccaacaagaatccagataatcccaagaaaaccacaaccctgtcctacgacgtgtataaggataagaggttttctgaggaccagtacgagctgcacatcccaatcgccatcaataagtgccccaagaacatcttcaagatcaatacagaggtgcgcgtgctgctgaagcacgacgataacccctatgtgatcggcatcgccaggggcgagcgcaatctgctgtatatcgtggtggtggacggcaagggcaacatcgtggagcagtattccctgaacgagatcatcaacaacttcaacggcatcaggatcaagacagattaccactctctgctggacaagaaggagaaggagaggttcgaggcccgccagaactggacctccatcgagaatatcaaggagctgaaggccggctatatctctcaggtggtgcacaagatctgcgagctggtggagaagtacgatgccgtgatcgccctggaggacctgaactctggctttaagaatagccgcgtgaaggtggagaagcaggtgtatcagaagttcgagaagatgctgatcgataagctgaactacatggtggacaagaagtctaatccttgtgcaacaggcggcgccctgaagggctatcagatcaccaataagttcgagagctttaagtccatgtctacccagaacggcttcatcttttacatccctgcctggctgacatccaagatcgatccatctaccggctttgtgaacctgctgaaaaccaagtataccagcatcgccgattccaagaagttcatcagctcctttgacaggatcatgtacgtgcccgaggaggatctgttcgagtttgccctggactataagaacttctctcgcacagacgccgattacatcaagaagtggaagctgtactcctacggcaaccggatcagaatcttccggaatcctaagaagaacaacgtgttcgactgggaggaggtgtgcctgaccagcgcctataaggagctgttcaacaagtacggcatcaattatcagcagggcgatatcagagccctgctgtgcgagcagtccgacaaggccttctactctagctttatggccctgatgagcctgatgctgcagatgcggaacagcatcacaggccgcaccgacgtggattttctgatcagccctgtgaagaactccgacggcatcttctacgatagccggaactatgaggcccaggagaatgccatcctgccaaagaacgccgacgccaatggcgcctataacatcgccagaaaggtgctgtgggccatcggccagttcaagaaggccgaggacgagaagctggataaggtgaagatcgccatctctaacaaggagtggctggagtacgcccagaccagcgtgaagcacaaaaggccggcggccacgaaaaaggccggccaggcaaaaaagaaaaagggatcctacccatacgatgttccagattacgcttatccctacgacgtgcctgattatgcatacccatatgatgtccccgactatgccggaagcgaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgtttta

SEQ ID NO: 5 - DNA, 5797 bp, Artificial Sequence
  Description of Artificial Sequence Synthetic polynucleotide
tggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgataaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttg

SEQ ID NO: 6 - DNA, 24 bp, Artificial Sequence
  Description of Artificial Sequence Synthetic oligonucleotide
gcgtaccatgaaaagtacccaacc

SEQ ID NO: 7 - DNA, 22 bp, Artificial Sequence
  Description of Artificial Sequence Synthetic oligonucleotide
tgctcagttgaagcttggcatc

SEQ ID NO: 8 - DNA, 5799 bp, Artificial Sequence
  Description of Artificial Sequence Synthetic polynucleotide
atggacaagaagtactccattgggctcgctatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgataaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggctgctatcgtgccccagtcttttctcaaagatgattctattgataataaagtgttgacaagatccgataaagctagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtcgccagggatccgtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctccgaaaaagaaacgcaaagttggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttga

SEQ ID NO: 9 - DNA, 26 bp, Artificial Sequence
  Description of Artificial Sequence Synthetic oligonucleotide
agagaacgtatgtcgagtttatccct

SEQ ID NO: 10 - DNA, 22 bp, Artificial Sequence
  Description of Artificial Sequence Synthetic oligonucleotide
cacccttggtcaccttcagctt

SEQ ID NO: 11 - DNA, 1498 bp, Artificial Sequence
  Description of Artificial Sequence Synthetic polynucleotide
aatattttgttaaaattggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttcgactcctctgcagaatgcggcgatgtttcggtaaggggtccgctatctagacgagtttactccctatcagtgatagagaacgtatgtcgagtttactccctatcagtgatagagaacgatgtcgagtttactccctatcagtgatagagaacgtatgtcgagtttactccctatcagtgatagagaacgtatgtcgagtttactccctatcagtgatagagaacgtatgtcgagtttatccctatcagtgatagagaacgtatgtcgagtttactccctatcagtgatagagaacgtatgtcgattaggcgtgtacggtgggcgcctataaaagcagagctcgtttagtgaaccgtcagatcgcctggagccaattccaactcttttgccttatacctactttccgtaccacttcctaccctcgaaaggcctctgaggccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaataggtgctgatgagtccgtgaggacgaaacgagtaagctcgtccacctactgcaggagatcgggttttagagctagaaatagcaagtt

SEQ ID NO: 12 - DNA, 4382 bp, Artificial Sequence
  Description of Artificial Sequence Synthetic polynucleotide
aaggcgggcctggcgggtctggtctccccatgcgggccaccagaggccctgcagccttcagtcgcttgaaggggtaatggcgcttccactcacaaacatggcggacagagcgtgtgaacgagatgaacagcccctcaaaaatatggccgccgaggctggacggccgtgccccagcagcaccgcctccgcgccccacgtgatctctcgccgggcacagcgctgaccgcggaggtccaaccggaagaatgtccggattggacattcggaagagggcccgccttccctggggaatctctgcgcacgcgcagaacgcttcgaccaatgaaaacacaggaagccgtccgcgcaaccgcgttgcgtcacttctgccgcccctgtttcaaggtatatagccgtagacggaacttcgcctttctctcggccttagcgccatttttttgggtgagtgttttttggttcctgcgttgggattccgtgtacaatccatagacatctgacctcggcacttagcatcatcacagcaaactaactgtagcctttctctctttccctgtagaaacctctgcacctgaggccaccatgtctagactggacaagagcaaagtcataaacggcgctctggaattactcaatggagtcggtatcgaaggcctgacgacaaggaaactcgctcaaaagctgggagttgagcagcctaccctgtactggcacgtgaagaacaagcgggccctgctcgatgccctgccaatcgagatgctggacaggcatcatacccacttctgccccctggaaggcgagtcatggcaagactttctgcggaacaacgccaagtcattccgctgtgctctcctctcacatcgcgacggggctaaagtgcatctcggcacccgcccaacagagaaacagtacgaaaccctggaaaatcagctcgcgttcctgtgtcagcaaggcttctccctggagaacgcactgtacgctctgtccgccgtgggccactttacactgggctgcgtattggaggaacaggagcatcaagtagcaaaagaggaaagagagacacctaccaccgattctatgcccccacttctgagacaagcaattgagctgttcgaccggcagggagccgaacctgccttccttttcggcctggaactaatcatatgtggcctggagaaacagctaaagtgcgaaagcggcgggccggccgacgcccttgacgattttgacttagacatgctcccagccgatgcccttgacgactttgaccttgatatgctgcctgctgacgctcttgacgattttgaccttgacatgctccccgggtccggcgctactaacttcagcctgctgaagcaggctggtgacgtcgaggagaatcctggtcccatggccatgcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaattcgaaggcctgtcgtgaagcttggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggcctactagctactcgggaccccttaccgaaacatcgccgcattctgcagaggagtcgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgtt

SEQ ID NO: 13 - DNA, 25 bp, Artificial Sequence
  Description of Artificial Sequence Synthetic oligonucleotide
gaaagccatgtggcctgaggagtga

SEQ ID NO: 14 - DNA, 50 bp, Artificial Sequence
  Description of Artificial Sequence Synthetic oligonucleotide
cgactttacgcttctttttaggcatgcagaggtttctacagggaaagaga

SEQ ID NO: 15 - DNA, 50 bp, Artificial Sequence
  Description of Artificial Sequence Synthetic oligonucleotide
cattgccgtcgaagccggaatctaagggtccggcgctactaacttcagcc

SEQ ID NO: 16 - DNA, 50 bp, Artificial Sequence
  Description of Artificial Sequence Synthetic oligonucleotide
cagaatcaggggataacgcaggaaagaacatgtgagcaaaaggccagcaa

SEQ ID NO: 17 - DNA, 25 bp, Artificial Sequence
  Description of Artificial Sequence Synthetic oligonucleotide
ggaccccttaccgaaacatcgccgc

SEQ ID NO: 18 - DNA, 1070 bp, Artificial Sequence
  Description of Artificial Sequence Synthetic polynucleotide
tcactcctcaggccacatggctttcttgtcctaggtagcctttagtccatgcgctgtaggtagcctttagtccatgggatcggccgttaggtagcctttagtccatgcaccgtaggtagcctttagtccatgttggctaggtagcctttagtccatgaagcttagacactagagggtatataatggaagctcgacttccagcttggcaatccggtactgttggtaaaggcctctgaggccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaataggtgctgatgagtccgtgaggacgaaacgagtaagctcgtccacctactgcaggagatcgggttttagagctagaaatagcaagttaaaataaggctagtccgttatcaacttgaaa

SEQ ID NO: 19 - DNA, 4991 bp, Artificial Sequence
  Description of Artificial Sequence Synthetic polynucleotide
ccattataagctgcaataaacaagttaacaacaacaattgcattcattttatgtttcaggttcagggggagatgtgggaggttttttaaagcaagtaaaacctctacaaatgtggtacctaggcgagaccctgtctcacaaaataaagtaagcccggactgagtgcggaaaggcgggcctggcgggtctggtctccccatgcgggccaccagaggccctgcagccttcagtcgcttgaaggggtaatggcgcttccactcacaaacatggcggacagagcgtgtgaacgagatgaacagcccctcaaaaatatggccgccgaggctggacggccgtgccccagcagcaccgcctccgcgccccacgtgatctctcgccgggcacagcgctgaccgcggaggtccaaccggaagaatgtccggattggacattcggaagagggcccgccttccctggggaatctctgcgcacgcgcagaacgcttcgaccaatgaaaacacaggaagccgtccgcgcaaccgcgttgcgtcacttctgccgcccctgtttcaaggtatatagccgtagacggaacttcgcctttctctcggccttagcgccatttttttgggtgagtgttttttggttcctgcgttgggattccgtgtacaatccatagacatctgacctcggcacttagcatcatcacagcaaactaactgtagcctttctctctttccctgtagaaacctctgcatgcctaaaaagaagcgtaaagtcgcccccccgaccgatgtcagcctgggggacgagctccacttagacggcgaggacgtggcgatggcgcatgccgacgcgctagacgatttcgatctggacatgttgggggacggggattccccggggccgggatttaccccccacgactccgccccctacggcgctctggatatggccgacttcgagtttgagcagatgtttaccgatgcccttggaattgacgagtacggtggggaattcggggcagacgacacacgcgttgaggtgcaaccgccggcgcagtgggtcctcgacctgatcgaggccagcccgatcgcatcggtcgtgtccgatccgcgtctcgccgacaatccgctgatcgccatcaaccaggccttcaccgacctgaccggctattccgaagaagaatgcgtcggccgcaattgccgattcctggcaggttccggcaccgagccgtggctgaccgacaagatccgccaaggcgtgcgcgagcacaagccggtgctggtcgagatcctgaactacaagaaggacggcacgccgttccgcaatgccgtgctcgttgcaccgatctacgatgacgacgacgagcttctctatttcctcggcagccaggtcgaagtcgacgacgaccagcccaacatgggcatggcgcgccgcgaacgcgccgcggaaatgctcaggacgctgtcgccgcgccagctcgaggttacgacgctggtggcatcgggcttgcgcaacaaggaagtggcggcccggctcggcctgtcggagaaaaccgtcaagatgcaccgcgggctggtgatggaaaagctcaacctgaagaccagtgccgatctggtgcgcattgccgtcgaagccggaatctaagggtccggcgctactaacttcagcctgctgaagcaggctggtgacgtcgaggagaatcctggtcccatggccatgcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaattcgaaggcctgtcgtgaagcttggggatcaattctctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggcctactagctactcgggaccccttaccgaaacatcgccgcattctgcagaggagtcgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttcgactcctctgcagaatgcggcgatgtttcggtaaggggtcc

SEQ ID NO: 20 - DNA, 166 bp, Artificial Sequence
  Description of Artificial Sequence Synthetic polynucleotide
agagaacgtatgtcgagtttactccctatcagtgatagagaacgtatgtcgattaggcgtgtacggtgggcgcctataaaagcagagctcgtttagtgaaccgtcagatcgcctggagccaattccaactcttttgccttatacctactttccgtaccacttccta

SEQ ID NO: 21 - DNA, 130 bp, Artificial Sequence
  Description of Artificial Sequence Synthetic polynucleotide
ctcgaaaggcctctgaggccatggtgagcaagggcgaggaggataacatggccatcatcaaggagttcatgcgcttcaaggtgcacatggagggctccgtgaacggccacgagttcgagatcgagggcga

SEQ ID NO: 22 - DNA, 26 bp, Artificial Sequence
  Description of Artificial Sequence Synthetic oligonucleotide
gcgagggccgcccctacgagggcacc

(19) United States
(12) Patent Application Publication
(10) Pub. No.: US 2021/0047653 A1
(43) Pub. Date: Feb. 18, 2021

(54) COMPOSITIONS AND METHODS FOR REGULATING A BIOLOGICAL PROCESS

(71) Applicant: The University of Memphis Research Foundation, Memphis, TN (US)

(72) Inventors: Gary L. Bowlin, Memphis, TN (US); Diego Augusto Velasquez Pulgarin, Memphis, TN (US); Alexander Espinosa, Memphis, TN (US)

(73) Assignee: The University of Memphis Research Foundation, Memphis, TN (US)

(21) Appl. No.: 16/963,809

(22) PCT Filed: Jan. 29, 2019
(86) PCT No.: PCT/US2019/015654

§ 371 Date: Jul. 21, 2020

Related U.S. Application Data
(60) Provisional application No. 62/623,954, filed on Jan. 30, 2018.

(51) Int. Cl. C12N 15/85; C12N 9/22; C12N 15/113

(52) U.S. Cl. CPC C12N 15/85; C12N 9/22; C12N 2310/20; C12N 2800/107; C12N 15/113

(57) ABSTRACT

The invention features compositions and methods for the inducible regulation of one or more target genes using a CRISPR-based synthetic gene regulatory network that responds to spatiotemporally-controlled agents present, for example, on a substrate (e.g., an electrospun template).

### CROSS-REFERENCE TO RELATED APPLICATIONS

This application claims the benefit of and priority to the following U.S. Provisional Application No. 62/623,954, filed Jan. 30, 2018, the entire contents of which are incorporated herein by reference.

### STATEMENT OF RIGHTS TO INVENTIONS MADE UNDER FEDERALLY SPONSORED RESEARCH

This invention was made with government support under Grant Number F31AR072502 awarded by the National Institutes of Health. The government has certain rights in the invention.

### BACKGROUND OF THE INVENTION

Notwithstanding the inherent regenerative capability of bone tissue, an estimated 100,000 fractures develop nonunion in the United States every year. Fracture nonunions are cases where the healing process is delayed, and are associated with prolonged disability and pain for the patient, while significantly increasing medical care costs. Nonunion incidence varies by anatomic region: tibia fractures, the most common long bone fracture, present nonunion at a rate of around 4%; humerus fractures at around 10-15% and femur fractures at around 6%. While relatively rare, the incidence of nonunion is related to high energy-trauma, rates of which have increased in the past decades in both civilian and military populations. Furthermore, the difficulty of treatment that leads to increased direct and indirect costs, along with increased use of medications (including opioids and Nonsteroidal anti-inflammatory drugs (NSAIDS)), makes nonunions a significant problem.

Nonunions are typically treated by mechanical fixation with or without biological stimulation. The gold standard biological stimulation technique is autogenous cancellous bone grafting. The autograft includes cells and biological factors (growth factors, extracellular matrix, etc.) that stimulate the local biological environment and increase the osteoinductive and osteoconductive properties of the graft. Despite autografts being effective, limited availability, donor site morbidity, and complications are associated drawbacks that have driven the development of alternatives. Among these alternatives, allografts, bone marrow injections, synthetic materials with osteoconductive properties, and recombinant growth factors have been approved and used clinically. Allografts present higher infection rates, and synthetic materials without biological stimulation do not perform satisfactorily. Recombinant growth factors have been efficacious nonunion treatment alternatives, particularly the bone morphogenetic proteins (BMPs), inducing a series of events leading to chondro-osteogenesis. BMP-2, BMP-7 and BMP-9 are the most potent of the BMPs, in terms of osteoinduction, but presently, only recombinant human BMP-2 (rhBMP-2) and BMP-7 (rhBMP-7) are commercially available and routinely used for spine fusion and nonunion fracture corrective surgical procedures. BMP-2 has been shown to reduce fracture healing times significantly in retrospective clinical studies.

Recombinant protein therapy, despite being efficacious, has been linked to adverse effects, such as ectopic bone growth, inflammatory responses, and cancerous cell formation. These adverse effects can be linked to the challenges of delivering recombinant proteins in appropriate dose and time profiles, short half-life of the growth factors, and supra-physiological doses required to compensate for reduced bioactivity of exogenous growth factors. In addition, recombinant protein therapy is costly, a barrier that limits availability to patients. Recombinant protein delivery methods are an active field of study, and advances have been made in development of carriers that address the difficulties of delivering effective doses in correct profiles for nonunion treatment. Nonetheless, cost and supra-physiological doses remain a hurdle to safe and effective nonunion therapy with exogenous recombinant proteins.

### SUMMARY OF THE INVENTION

There is a need for precisely regulated, localized, spatiotemporally-controlled delivery of proteins in tissue engineering and regenerative medicine. Many of the associated complications of recombinant protein therapy (exogenously produced and by gene therapy) are circumvented using synthetic gene-regulatory-networks (GRNs) with endogenous proteins as outputs. The use of the invention can lead to reduced complications and improved outcomes for patients as well as reduced time to healing/regeneration of tissues, such as bone.

As described below, the present invention features compositions and methods for the inducible regulation of one or more target genes using a CRISPR-based synthetic gene regulatory network (GRNs) that responds to spatiotemporally-controlled agents present, for example, on a substrate (e.g., an electrospun template).

In particular embodiments, nucleolytically-inactive CRISPR-associated proteins (Cas), like dCas9 and dCpf1 (but not limited to only these), with activation/repression domains fused to their N-terminus (e.g. VPR, SAM, KRAB, etc.) and sequence-specific gRNA, the logic gates used to build the synthetic gene regulatory networks can be layered, combined, modified, and multiplexed to target and modulate any endogenous or recombinant protein. These elements are used to precisely regulate the spatiotemporal pattern of protein delivery in tissue engineering and regenerative medicine. In one embodiment, an electrospun template is used to deliver physicochemical cues to a cell (e.g., stem cell, fibroblast, cartilage-derived cell, bone-derived cell, adipose-derived stem cells) to generate a gradient of growth factors that guide regeneration. In particular embodiments, adipose-derived stem cells are engineered to express a synthetic gene regulatory network (GRN) constructed with CRISPR-based logic gates, allowing them to respond to stimuli presented by the template. In particular embodiments, a cell of the invention comprises inducible nucleolytically-inactive CRISPR-associated proteins (such as dCas9 and dCpf1) and gRNAs delivered by transposons to target endogenous genes encoding one or more proteins of interest (e.g., BMP-2 and PDGF-BB).

The constructs for dCas9, dCpf1, and gRNAs are inducible by an agent (e.g., doxycycline, cumate, blue light). The use of dCas9 as transcription activators and Cpf1, a recently described CRISPR-associated protein using different gRNA types and PAM, as transcription repressors, allows orthogonality in the system and the generation of logical gates forming the GRN. In particular, dCpf1 is used as a transcription repressor.

In one particular example, tissue regeneration is carried out to generate a specialized interface referred to as an enthesis. The enthesis is defined by the insertion of tendons/ligaments into bone. It is a highly-organized tissue presenting continuous gradients of structural and mechanical properties that allow smooth force transfer, protecting and maintaining the tendon/ligament insertion. An electrospun template is used to deliver physicochemical cues to engineered adipose-derived stem cells (ASCs) to generate a countergradient of growth factors that will guide the regeneration of the structural and functional characteristics of the enthesis. In particular, an electrospun template is used to provide countergradients of growth factors linked to differentiation to osteoblastic (BMP-2) and ligamentocyte/tenocyte (PDGF-BB) lineages. The stimuli present on one end of the template will include both cumate and blue light, leading to BMP-2 production. On the opposite end of the template, only cumate will be detected by the ASCs, leading to PDGF-BB production. The ability of the engineered ASCs to respond to stimuli and deliver endogenously produced growth factors in a spatiotemporally controlled manner circumvents supra-physiological dosages clinically used in tissue engineering with recombinant growth factors. The circuit will allow the cells to express the product of interest only while in proximity to the template, but function as normal cells in the absence of the effectors, mitigating ectopic action. The engineered cell approach for delivery of these growth factors also addresses the challenge of prolonged, local delivery. A schematic for an exemplary gene regulatory network (GRN) is provided in FIG. 7.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “a nucleolytically-inactive CRISPR-associated (Cas) protein” is a Cas that has mutations in its nuclease domains (e.g. RuvC and HNH nuclease domain in the case of Cas9) that destroy its capability to cleave nucleic acids. All other functions of the Cas remain intact. These nucleolytically-inactive Cas are annotated with a lower case “d” in front of their name (e.g. dCas9), denoting catalytically “dead” proteins.

By “agent” is meant a peptide, nucleic acid molecule, small compound, or stimulus. Agents include, for example, cumate, light (e.g., blue light), doxycycline.

By “ameliorate” is meant decrease, suppress, attenuate, diminish, arrest, or stabilize the development or progression of a disease or disorder.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of the analyte to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include a failure in tissue regeneration. In one embodiment, the invention ameliorates a failure of enthesis regeneration.

By “effective amount” is meant the amount of a required to ameliorate the symptoms of a disease relative to an untreated patient. The effective amount of a cell, tissue or organ generated using a system of the invention is that amount needed for the therapeutic treatment of a disease. An effective amount varies depending upon the manner of administration, the age, body weight, and general health of the subject. Ultimately, the attending physician or veterinarian will decide the appropriate amount and dosage regimen. Such amount is referred to as an “effective” amount.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

As used herein, “obtaining” as in “obtaining an agent” includes synthesizing, purchasing, or otherwise acquiring the agent.

By “reduces” is meant a negative alteration of at least 10%, 25%, 50%, 75%, or 100%.

By “reference” is meant a standard or control condition.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, preferably at least about 20 amino acids, more preferably at least about 25 amino acids, and even more preferably about 35 amino acids, about 50 amino acids, or about 100 amino acids. For nucleic acids, the length of the reference nucleic acid sequence will generally be at least about 50 nucleotides, preferably at least about 60 nucleotides, more preferably at least about 75 nucleotides, and even more preferably about 100 nucleotides or about 300 nucleotides or any integer thereabout or therebetween.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100·mu·g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptom associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 provides dCas9-VPR sequence. This is the nucleolitically-inactive Cas9 with a VP64-p65-rta (VPR) fusion. This is cloned into the MCS of the cumate inducible piggybac transposon (2).

FIG. 2 is a PB-Cuo-MCS-IRES-GFP-EF1a-CymR-Puro plasmid map, a Cumate-inducible piggybac transposon plasmid map.

FIG. 3 provides pSBtet-HHgRNAHDV-mCherry sequence. This is the doxycycline-inducible sleeping beauty transposon.

FIG. 4 provides a pSBtet-HHgRNAHDV-mCherry plasmid map.

FIG. 5 provides a SB-EL222-HH-gRNAb-HDV-mCherry sequence. This is the blue light-inducible sleeping beauty transposon.

FIG. 6 provides a SB-EL222-HH-gRNAb-HDV-mCherry plasmid map.

FIG. 7 provides a schematic diagram of a proposed gene regulatory network (GRN).

FIG. 8 provides a schematic representation of CRISPR-based AND gate network comprising vector A and vector B. Vector A expresses dCas9, whose expression is under external stimulation. dCas9 complexes with gRNA expressed by vector B. This complex of dCas9/gRNA activates the transcription and expression of the target gene (BMP-2).

FIG. 9 provides the restriction analysis with EcoNI of vector pSBtet-HH-gRNA-HDV-mCherry indicates correct cloning of insert. The white and grey arrows point to the 8 Kb and 3 Kb markers respectively, regions where bands were expected in correctly cloned vector. pSBtet-Bla is empty vector used as negative control.

FIG. 10 provides the sequence alignment of pSBtet-HH-gRNA-HDV-mCHerry with reference plasmid sequence suggests correct cloning and orientation of the insert. Area highlighted is the junction between vector and insert and includes SfiI recognition sites.

FIG. 11 provides the results of a PCR screen for bacterial colonies transformed with pPBq-dCas9-VPR show successful cloning of insert. PCR primers were targeted to the insert and presence of the PCR product was seen in 16 of 17 colonies. The arrows indicate colonies selected for miniprep and downstream applications.

FIG. 12 provides the restriction analysis with NotI and NheI of pPBq-dCas9-VPR suggests correct cloning of inserts. The arrows indicate 10 Kb and 6 Kb markers, were bands were expected in correctly cloned plasmids.

FIG. 13 provides the fluorescence microscopy analysis of pSBtet-HH-gRNA-HDV-mCherry expression. The analysis suggests correct function of inducible SB transposon. The presence of mCherry in the SB cells after 24 hours of stimulation with 1 μM doxycycline is indicative of activation of the SB transposon, while lack of mCherry in the WT and SB cells without stimulation suggest no activation of the SB transposon.

FIG. 14 provides the FACS results for pPBq-dCas9-VPR activation. The results suggest EGFP is coexpressed with dCas9-VPR under cumate stimulation in a regulated, titratable fashion. The x-axis shift progression from WT, to PB no stimulation, PB 1× cumate stimulation, and PB 10× stimulation is indicative of proper cumate switch function and low background expression.

FIG. 15 provides the fluorescence microscopy analysis of pSBtet-HH-gRNA-HDV-mCherry and pPBq-dCas9-VPR co-expression. The analysis indicates correct function of inducible SB and PB transposons. The presence of mCherry and EGFP in the SB/PB cells after 24 hours of stimulation with 1 μM doxycycline and 10× cumate is indicative of activation of both transposons, while lack of mCherry and EGFP in the WT and SB/PB cells without stimulation suggest no activation of the SB and PB transposons.

FIG. 16 provides the luciferase expression assay for synthetic promoter reporter AND gate function. The results here suggest non-specific activation of the luciferase gene under doxycycline stimulation. SB/PB cells under cumate stimulation show no luciferase activity, whereas the SB/PB cells under doxycycline and doxycycline/cumate stimulation show increased luciferase activity. This behavior is not congruent with proper AND gate function.

FIG. 17 provides the luciferase expression assay for BMP-2 promoter reporter AND gate function. The results here indicate no activation of the luciferase gene under stimulation. SB/PB cells under doxycycline, cumate, and doxycycline/cumate stimulation show no luciferase activity. This behavior is not congruent with proper AND gate function.

FIG. 18 provides a micrograph of rat supraspinatus enthesis¹.

FIG. 19 provides the structure and composition of a typical ligament/tendon enthesis².

FIG. 20 provides the counter gradient of Scx and Sox9 in developing enthesis².

FIG. 21 provides the FACS results for P B-Q-dCas9-VPR.

FIG. 22 provides the luciferase expression assay for pSB-TRE-gRNA1-Bla stimulated with doxycycline.

FIG. 23 provides the luciferase assay results for CRISPR AND gate.

FIG. 24 provides representative fluorescence microscopy analysis of pSBtet-HH-gRNA-HDV-mCherry and pPBq-dCas9-VPR co-expression. The analysis indicates correct function of inducible SB and PB transposons.

FIG. 25 provides representative fluorescence microscopy analysis of pSBtet-HH-gRNA pSBtet-HH-gRNA-HDV-mCherry and pPBq-dCas9-VPR co-expression. GFP and mCherry overlay.

FIG. 26 provides the luciferase expression assay for BMP-2 promoter reporter AND gate function. The results indicate no activation of the luciferase gene under stimulation. SB/PB cells under doxycycline, cumate, and doxycycline/cumate stimulation show no luciferase activity. This behavior is not congruent with proper AND gate function. (n=3).

FIG. 27 demonstrates the induction of the expression of endogenous BMP-2 as normalized to the HPRT expression.

FIG. 28 shows the induction of the expression of endogenous BMP-2 compared to induction of the expression of GFP by GFP-targeting gRNA.

FIG. 29 shows a representative scanning electromicrograph (SEM) of an air gap electrospun template.

FIG. 30 shows a representative force-elongation curve of an air gap electrospun template.

FIG. 31 shows the blue light gradient image and pixel intensity plot along a template longitudinal axis.

FIG. 32 shows the Gating strategy and fluorescence measurements of wild type HEK-293T cells.

FIG. 33 shows the Gating strategy and fluorescence measurements of unstimulated pSBtet-mCherry-RGR-Bla transfected HEK-293T cells.

FIG. 34 shows the Gating strategy and fluorescence measurements of stimulated (1 μM Doxycycline) pSBtet-mCherry-RGR-Bla transfected HEK-293T cells, 18 hours post-stimulation.

FIG. 35 shows the comparison of fluorescence measurements of Wild Type, unstimulated, and stimulated (1 μM Doxycycline) pSBtet-mCherry-RGR-Bla transfected HEK-293T cells, 18 hours post-stimulation.

### DETAILED DESCRIPTION OF THE INVENTION

As described below, the present invention features compositions and methods for the inducible regulation of one or more target genes using a CRISPR-based synthetic gene regulatory network (GRNs) that responds to spatiotemporally-controlled agents present, for example, on a substrate (e.g., an electrospun template).

The invention is based, at least in part, on the discovery of a system for spatially and temporally regulating the transcription of one or more target genes in a cell. In particular, the invention features two inducible constructs encoding for a gRNA and dCas9, responding to doxycycline and cumate, respectively. The CRISPR-based synthetic GRN uses a nucleolytically-inactive CRISPR-associated (Cas) protein that has mutations in its nuclease domains that destroy its capability to cleave nucleic acids, i.e., the endonuclease activity is destroyed. All other functions of the Cas remain intact. Only in the presence of both stimuli will gRNA bind to its target DNA and recruit dCas9 to activate transcription of a target endogenous gene (e.g., BMP-2). Both constructs were cloned into transposons, as this system allows for large payloads and is usually less susceptible to epigenetic silencing. HEK-293T cells were transfected with both constructs to create stable cell lines with a gene regulatory network (GRN) to control production of endogenous BMP-2 in response to external stimuli. These engineered cells with synthetic GRN are able to produce highly regulated, localized, and bioactive BMP-2 while exhibiting a response behavior characteristic of an AND gate.

In some embodiments, dCas9, dCPF1, and other nucleolytically-inactive CRISPR-associated proteins are useful for construction the GRNs. In some embodiments, any Type II CRISPR Cas9 (from different species, like S. pyogenes, S. thermophiles, N. meningitidis, S. aureus, and F. novicida), and Type V CRISPR Cas (Cpf1 (also known as Cas12a), different species, like Prevotella, Francisella can be for constructing the GRNs.

In some embodiments of various aspects described herein, the activation domain in the GRNs includes but limited to SunTag, synergistic tripartite activator domain, and the multiple VP16 (Herpes simplex virus protein vmw65) activator domains.

In some embodiments of various aspects described herein, the repressor domain in the GRNs includes but limited to KRAB. In one embodiment, just dCas or dCPF1 alone without a repressor domain can sterically inhibit transcription, and serve to repress transcription in a GRN.

In some embodiments of various aspects described herein, the gRNA is expressed on a separate construct, and also be expressed from the same construct, just under control of a separate inducible promoter.

The present invention provides methods of treating disease and/or disorders or symptoms thereof which comprise providing to the patient a system to induce in vivo spatiotemporally regulated tissue regeneration or to provide to the patient an in vitro or ex vivo tissue regenerated using the compositions and/or methods of the invention. Thus, one embodiment is a method of treating a subject suffering from or susceptible to a defect in tissue regeneration or symptom thereof. The method includes the step of administering to the mammal a therapeutic amount of tissue or providing a system for tissue regeneration as described herein sufficient to treat the disease or disorder or symptom thereof, under conditions such that the disease or disorder is treated.

The methods herein include administering to the subject (including a subject identified as in need of such treatment) an effective amount of the compositions described herein (e.g., engineered cells, tissues, and the like), or a system of inducible vectors and other compositions (e.g., electrospun templates) described herein to produce spatiotemporally regulated tissue regeneration. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to promoting tissue regeneration or otherwise regulating a biological process using the compositions and methods of the invention. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of a tissue or compositions described herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” is made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which defects in tissue regeneration or another biological process may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with defects in tissue regeneration or another biological process, in which the subject has been administered a therapeutic amount of a tissue or vector described herein as sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Templates (Also Termed “Scaffolds”)

Electrospun materials possess a nanoscale fibrous structure that mimics the extracellular matrix (ECM) to support cell attachment and growth, and provides the advantage of having increased surface area for drug delivery. In vivo, the ECM is a three-dimensional network of extracellular macromolecules, such as collagen, enzymes, and glycoproteins, that provide structural and biochemical support of surrounding cells.

The disclosed nanofiber structure also results in interconnected porous structures, allowing communication between tissue compartments. This communication between cells is a vital component of the natural healing response, as chemotactic factors help native cells located in and around the site migrate into the template.

The invention provides a diverse set of agent-loaded templates. In one embodiment, an agent of the invention is used to control (e.g., induce) the expression of a gene of interest. Accordingly, agent-loaded templates are loaded, for example, with cumate, doxycycline or any other agent useful for regulating promoter expression. In one embodiment, the template is loaded with an agent that provides for induction by blue light. Both positive and negative polarities are applied to the electrospinning polymer solution, opposite a grounded collection mandrel, to create an electrical field driving force. In one embodiment, these parameters are optimized to modify the resultant drug-release profile of the template. Polymer concentration, which informs fiber diameter, is optimized to alter porosity, SAVR, and drug loading parameters.

In general, the materials of the invention comprise a biodegradable polymer and, if desired, a filler. A variety of biodegradable polymers are known in the art. Preferred biodegradable polymers include proteins (such as gelatin and collagen), polymers derived from naturally-occurring monomers (such as poly(lactic acid (PLA)), and polymers derived from synthetic monomers (such as polydioxanone (PDO)). Desirably, biodegradable materials will degrade over a time period of less than a year, more preferably less than six months. In general, any biodegradable polymer that is biocompatible, and is shaped or formed into fibers and membranes, is employed in the present materials. Copolymers or mixtures/blends (multi-component) of biodegradable polymers can also be employed.

Other biocompatible polymers, some of which are biodegradable, include, e.g., Such polymers include but are not limited to the following: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO) and polyorthoesters or any other similar synthetic polymers that may be developed that are biologically compatible. Some preferred synthetic matrix materials include PEA, PGA, copolymers of PLA and PGA, polycaprolactone, poly(ethylene-co-vinyl acetate), (EVOH), PVA, and PEO, See also U.S. Pat. No. 7,374,774 (which is incorporated herein by reference).

The term “filler”, as used herein, refers to an organic or inorganic biocompatible material that provides structural reinforcement or rigidity to a polymer fiber, filament, or membrane. The filler may be a crystalline, a fiber, or a particle. Alternatively, the filler suitably has a shape of rod, fiber, sphere, oval, polyhedral crystal, and the like, however, the shape of the filler is not particularly limited thereto. The filler has an average diameter in nanoscale (nanofiller) ranging from about 1 nm to about 950 nm. The nanofiller suitably has an average diameter of about 1-100 nm, of about 10-80 nm, of about 25-75 nm, or particularly of about 50 nm. Alternatively, the filler has an average diameter in microscale (microfiller) that is greater than at least about 100 nm. The microfiller suitably has an average diameter of about less than about 10 micron, less than about 9 micron, less than about 8 micron, less than about 7 micron, less than about 6 micron, less than about 5 micron, less than about 4 micron, less than about 3 micron, less than about 2 micron, or particularly less than about 1 micron. For example, the filler is a nanocrystalline or fiber material and has an average diameter or thickness of less than about 100 nm, and advantageously may have an average length of less than about 500 nm. Advantageously, a nanofiller can possess an electrostatic charge, which may adhere to or attract growth factors when implanted or applied to a wound site.

### Methods for Preparing Compositions

Compositions comprising a biodegradable polymer, a filler, and an agent is prepared by any suitable method, some of which are known in the art. In general, a filler is suspended or dispersed in a solvent (which will not substantially dissolve the filler) to form a dispersion or suspension; the biodegradable polymer and the agent are then mixed with the dispersion or suspension to form a composition of the invention. In certain embodiment, a therapeutically effective amount of agent is additionally added to the composition for enhancing regeneration. In certain embodiments, the solvent is 2,2,2-trifluoroethanol, 1,1,1,3,3,3-hexafluoro-2-propanol (HFP) or 9:1 acetic acid:water. The amount of solvent used should be minimized to facilitate electrospinning or other processing of the composition into fibers and membranes.

### Methods for Preparing Fibers and Membranes

A composition comprising a biodegradable polymer, a filler, and an agent is used to prepare fibers and membranes by any suitable method, some of which are known in the art. In one embodiment, a fiber or membrane is formed by electrospinning. Electrospinning is a known technique (see, e.g., Li et al., Biomaterials. 2005 October; 26(30):5999-6008.) and electrospinning apparatus is purchased commercially. For example, a charged solution comprising, for example, a biodegradable polymer is fed through a small opening or nozzle (usually a needle or pipette tip). Due to its charge, the solution is drawn toward a grounded collecting plate, e.g., a metal screen, plate, or rotating mandrel, typically 5-30 cm away, as a jet. During the jet's travel, the solvent gradually evaporates, and a charged fiber is left to accumulate on the grounded target. The charge on the fibers eventually dissipates into the surrounding environment. If the target is allowed to move with respect to the nozzle position, specific fiber orientations (aligned or random) is achieved. For further examples, see U.S. Pat. Nos. 6,713,011, 6,616,435, and 7,029,620. The entire contents of which are incorporated herein by reference.

The compositions of the invention are made as electrospun fiber compositions. In one embodiment, the invention provides a method of producing a membrane, the method comprising:

a) dispersing a filler in a solvent to form a dispersion;

b) combining a biodegradable polymer and an agent with the dispersion to form a composition; and

c) electrospinning the composition to form fibers, thereby forming a membrane comprising a biodegradable polymer, a filler, and an effective amount of agent (e.g., cumate, doxycycline).

In certain embodiments, the filler is added to the composition, such that the step a) is omitted and the biodegradable polymer and agent is combined with the solvent to form a composition.

The method may further comprise adding at least one additional filler and at least one therapeutic agent before electrospinning. The electrospun membrane is formed in multiple layers. For example, the composition is additionally electrospun on top of one layer or other layers to create multiple-layer electrospun membrane.

In another embodiment, the solvent is removed from a dispersion comprising a biodegradable polymer, a filler, and an effective amount of agent to form a sponge. Solvent is removed by evaporation or lyophilization (freeze-drying). Thus, in one embodiment, the invention provides a method of producing a membrane. A membrane for use in the therapeutic methods of the invention should have sufficient rigidity to support the surrounding soft tissue, be malleable at its glass transition temperature (Tg) but regain rigidity on cooling (i.e. hold shape formed in situ), and be biocompatible in that it will promote osseointegration and not adversely affect the surrounding soft tissue. The membrane should resorb within 6-9 months as it takes approximately 6 months for allograft bone to consolidate into new bone in the mandible and maxilla bone graft surgeries. The membranes of the invention are flexible, moldable upon heating, maintain their shape upon cooling, are less acidic during degradation, and the fibrous architecture will allow for regeneration of bone and tissue.

The size and thickness of a membrane of the invention is varied according to the intended use. The membranes are spun to a desired size, or a sponge is cast to a desired size, followed by compression to a desired density and thickness. For example, barrier membranes are commonly between 0.1-0.4 mm in thickness, so the sponge can be suitably compressed to a thickness of about 0.1-0.4 mm.

The membrane can have any shape (round, square, rectangular, irregular). In exemplary embodiments, a membrane of the invention has a width from 1 to 20 mm and a length from 1 to 20 mm. In certain embodiments, a membrane is less than 1 mm in thickness, less than 0.5 mm thickness, less than 0.3 mm in thickness, or less than 100 microns in thickness.

### Therapeutic and Prophylactic Applications

The present invention provides a ready supply of materials useful for promoting tissue regeneration. Compositions and materials of the invention are administered (e.g., directly or indirectly) to a damaged or diseased tissue or organ where they engraft and establish functional connections with a target tissue (e.g., bone, muscle, ligament, tendon). In one embodiment, a membrane of the invention enhances bone healing. Methods for repairing damaged tissue or organs may be carried out either in vitro, in vivo, or ex vivo. In another embodiment, the invention provides a method of promoting bone regeneration, the method comprising contacting a bone surface with a composition, fiber, compressed membrane, particulate, swelling membrane, non-compressed membrane or multiple-layer membrane (compressed or non-compressed) of the invention. In certain embodiments, the method is a method of promoting bone regeneration after a surgical procedure on bone or ligament (e.g., ligament reconstructive surgery).

In another embodiment, the invention provides a method of promoting healing of a bone defect, the method comprising contacting the bone defect with a composition, fiber, compressed membrane, particulate, swelling membrane, non-compressed membrane or multiple-layer membrane (compressed or non-compressed) of the invention.

In still another embodiment, the invention provides a method of promoting soft tissue healing in a damaged tissue, the method comprising contacting the damaged tissue with a composition, fiber, membrane, particulate, swelling membrane, non-compressed membrane or multiple-layer membrane (compressed or non-compressed) of the invention.

In certain embodiments of the above aspects, the method is a method of promoting bone regeneration after a surgical procedure on bone or ligament.

### Administration

Compositions, fiber, and membranes of the invention can be provided directly to a tissue or organ of interest (e.g., by direct application to a bone or tissue surface, or by surgical implantation). A membrane can be applied to cover, surround, fill, or otherwise contact a bone or tissue defect, wound, skin/wound healing, or surgical site. If desired, expansion and differentiation agents can be provided prior to, during or after administration of the composition, fiber, or membrane to increase, maintain, or enhance production or differentiation of cells in vivo, including bone cells from a subject's bone or from any type of bone graft material/transplant, i.e., allogenic, xenogenic, alloplastic or genetically produced bone. Compositions of the invention include pharmaceutical compositions. When administering a therapeutic composition or material of the present invention (e.g., a pharmaceutical composition), it will generally be formulated in a unit dosage form. Agents, including additional therapeutic agents can be applied to the fibers or incorporated within fibers during fabrication.

### Formulations

Compositions, fibers, membranes, or multiple-layer membranes of the invention of the invention can be conveniently provided as sterile preparations. Sterile injectable solutions can be prepared by incorporating the cells (e.g., embryonic stem cells, neuronal progenitors, differentiated neurons) as desired. Such compositions may be in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. In addition, silver salts can be used as thickening agent. See also U.S. Pat. Nos. 8,367,094; 8,173,151; and 7,998,498 (which are incorporated herein by reference). The preferred concentration of the thickener will depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

Glycerin or similar components can be added to the admixture to improve fiber and membrane flexibility.

Exemplary agents that may be delivered together with a composition, fiber, membrane, or multiple-layer membrane of the invention of the invention include, but are not limited to, antibiotics, analgesics, anticoagulants, immunosuppressants, the therapeutic substance is selected from the group consisting of anesthetics, hypnotics, sedatives, sleep inducers, antipsychotics, antidepressants, antiallergics, antianginals, antiarthritics, antiasthmatics, antidiabetics, antidiarrheal drugs, anticonvulsants, antigout drugs, antihistamines, antipruritics, emetics, antiemetics, antispasmondics, appetite suppressants, neuroactive substances, neurotransmitter agonists, antagonists, receptor blockers, reuptake modulators, beta-adrenergic blockers, calcium channel blockers, disulfarim, muscle relaxants, analgesics, antipyretics, stimulants, anticholinesterase agents, parasympathomimetic agents, hormones, antithrombotics, thrombolytics, immunoglobulins, hormone agonists, hormone antagonists, vitamins, antineoplastics, antacids, digestants, laxatives, cathartics, antiseptics, diuretics, disinfectants, fungicides, ectoparasiticides, antiparasitics, heavy metals, heavy metal antagonists, chelating agents, alkaloids, salts, ions, autacoids, digitalis, cardiac glycosides, antiarrhythmics, antihypertensives, vasodilators, vasoconstrictors, antimuscarinics, ganglionic stimulating agents, ganglionic blocking agents, neuromuscular blocking agents, adrenergic nerve inhibitors, anti-oxidants, anti-inflammatories, wound care products, antitumoral agents, antiangiogenic agents, antigenic agents, wound healing agents, plant extracts, growth factors, growth hormones, cytokines, immunoglobulins, emollients, humectants, anti-rejection drugs, spermicides, conditioners, antibacterial agents, antifungal agents, antiviral agents, tranquilizers, cholesterol-reducing drugs, antitussives, histamine-blocking drugs and monoamine oxidase inhibitors.

Other agents include proteins such as any one or more of activin A, adrenomedullin, acidic FGF, basic fibroblast growth factor, angiogenin, angiopoietin-1, angiopoietin-2, angiopoietin-3, angiopoietin-4, angiostatin, angiotropin, angiotensin-2, bone morphogenic protein 1, 2, or 3, cadherin, collagen, colony stimulating factor (CSF), endothelial cell-derived growth factor, endoglin, endothelin, endostatin, endothelial cell growth inhibitor, endothelial cell-viability maintaining factor, ephrins, erythropoietin, hepatocyte growth factor, human growth hormone, TNF-alpha, TGF-beta, platelet derived endothelial cell growth factor (PD-ECGF), platelet derived endothelial growth factor (PDGF), insulin-like growth factor-1 or -2 (IGF), interleukin (IL)-1 or 8, FGF-5, fibronectin, granulocyte macrophage colony stimulating factor (GM-CSF), heart derived inhibitor of vascular cell proliferation, IFN-gamma, IFN-gamma, integrin receptor, LIF, leiomyoma-derived growth factor, MCP-1, macrophage-derived growth factor, monocyte-derived growth factor, MMP 2, MMP3, MMP9, neuropilin, neurothelin, nitric oxide donors, nitric oxide synthase (NOS), stem cell factor (SCF), VEGF-A, VEGF-B, VEGF-C, VEGF-D, VEGF-E, VEGF, and VEGF164. Other agents that may be delivered together with a cell of the invention include one or more of LIF, bone morphogenic protein (BMP), retinoic acid, trans-retinoic acid, dexamethasone, insulin, indomethacin, fibronectin and/or 10% fetal bovine serum, or a derivative thereof. Other agents include small oligonucleotides, such as SiDNA or SiRNA including at least a portion of sequences to a therapeutic target.

Those skilled in the art will recognize that the polymeric components of the compositions should be selected to be chemically inert and will not affect the viability or efficacy of the cell as described in the present invention. This will present no problem to those skilled in chemical and pharmaceutical principles, or problems can be readily avoided by reference to standard texts or by simple experiments (not involving undue experimentation), from this disclosure and the documents cited herein.

### Dosages

A composition, fiber, or membrane of this invention can be applied or implanted in an amount effective to provide tissue regenerating properties. The skilled artisan can readily determine the amount of the composition, fiber, or membrane of the invention to be administered in methods of the invention. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

### Delivery Methods

Compositions of the invention (e.g., scaffolds comprising agents or cells) can be provided directly to a tissue or organ of interest, such as a tissue damaged from injury or disease. Compositions can be administered to subjects in need thereof by a variety of administration routes. Methods of administration, generally speaking, may be practiced using any mode of administration that is medically acceptable, meaning any mode that produces effective levels of the active compounds without causing clinically unacceptable adverse effects. Such modes of administration include surgical engraftment or injection (e.g., intramuscular, intra-cardiac, intraocular, intracerebroventricular).

### Kits

Compositions, fibers, membranes, or multiple-layer membranes of the invention may be supplied along with additional reagents in a kit. The kits can include instructions for the preparation of a material (such as a membrane), a treatment regime, reagents, and equipment (test tubes, reaction vessels, needles, syringes, etc.). The instructions provided in a kit according to the invention may be directed to suitable operational parameters in the form of a label or a separate insert.

In one embodiment, compositions, fiber, membranes, or multiple-layer membranes of the invention are useful for the treatment or prevention of injury or disease of bone or soft tissue. The present invention provides compositions and methods of treating such injuries or diseases and/or symptoms thereof characterized by the loss of cells, or loss of tissue structure, function or activity. The methods of the invention comprise administering a therapeutically effective amount of a composition, fiber, membrane, or multiple-layer membrane described herein to a subject (e.g., a mammal, such as a human). Thus, one embodiment is a method of treating a subject suffering from or susceptible to a disease, condition or disorder characterized by the loss of cells, or loss of tissue structure, function or activity. The method includes the step of administering to the mammal a therapeutic amount of a characterized by the loss of cells, or loss of tissue structure, function or activity herein sufficient to treat the disease, condition, or disorder, or symptom thereof, under conditions such that the disease, condition, or disorder, or symptom thereof is treated.

The methods herein include administering to the subject (including a subject identified as in need of such treatment) an effective amount of a composition, fiber, membrane, or multiple-layer membrane described herein, to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compositions herein, such as a composition, fiber, membrane, or multiple-layer membrane described herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like).

The following examples are put forth so as to provide those of ordinary skill in the art with a complete disclosure and description of how to make and use the assay, screening, and therapeutic methods of the invention, and are not intended to limit the scope of what the inventors regard as their invention. The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Utilizing Inducible dCas9 and gRNA Expressing Constructs, a Circuit can be Assembled to Regulate Expression of an Endogenous Gene in Response to Stimuli, Emulating a Logic Gate

Gene therapy approaches that circumvent the exogenous recombinant protein bioactivity deficit by delivering nucleic acids encoding constitutively expressed BMP genes al low delivery of high local concentrations of endogenously produced BMP⁸. Approaches where cells are constitutively over expressing transgenes raise concerns, such as ectopic bone formation, cytotoxic concentrations of BMP, inability to control release profiles, and inability to suppress transgene expression in heterotopic sites⁸⁻¹⁰. The invention provides a Clustered Regularly Interspaced Short Palindromic Repeats (CRISPR)-based logic AND gate for the controlled delivery of endogenously produced BMP-2.

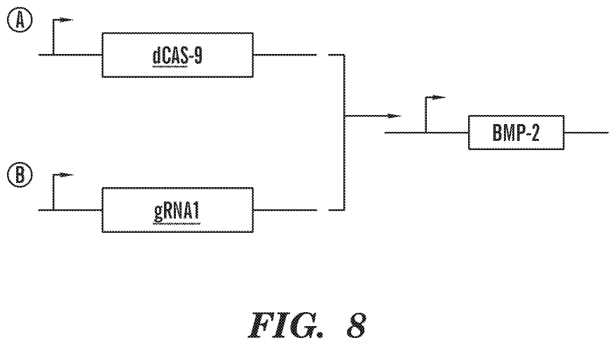

Engineered cellular production of growth factors with regulation of spatio-temporal gene expression would allow for highly effective treatments, minimized complications, and biomimetic dynamic gene expression profiles¹¹. Synthetic biology approaches that construct artificial gene regulatory networks (GRN) can provide tightly regulated growth factor production that responds to physico-chemical cues that are convenient to deliver and control¹². These GRN are designed in abstract, high level code, where sequential functions are aligned to generate desired outputs, and then are transformed to active biological circuit designs that are assembled and inserted into host genomes¹². A large portion of these biological circuits rely on transcription regulation mechanisms, and CRISPR/Cas9 systems have simplified the ability to target and regulate specific endogenous genes efficiently. CRISPR/Cas9 systems are nucleic acid-targeting, RNA-directed adaptive restriction machineries used by prokaryotes as defense mechanisms against mobile genetic elements¹³. Although the adaptation of these systems to genome editing has revolutionized genetic engineering, the technology has been used for other applications, including live-cell DNA imaging and transcriptional activation/repression¹⁴⁻¹⁶. CRISPR activation/repression is a simple approach to target gene expression control in the mammalian genome¹³. The use of guide RNA (gRNA)-guided nucleolytically-inactive CRISPR-associated proteins (dCas9) fused to transcriptional regulators is an efficient and cost-effective solution to targeting and regulating single or multiple genes¹³. This system improves on the targeting efficiency of RNA-interference approaches, and is simpler, less costly, and more readily deliverable into cells than protein-based tools like zinc finger and transcription-activator-like effector nucleases^{13,16}. In CRISPR activation/repression, a gRNA binds target DNA sequences, dictated by a designed 20 nucleotide complementary sequence adjacent to a protospacer-adjacent motif (PAM)¹³. The gRNA localizes and complexes a dCas9 protein that activates/represses gene expression¹³. By utilizing inducible dCas9 and gRNA expressing constructs, a circuit can be assembled to regulate expression of an endogenous gene in response to stimuli, emulating a logic gate. A schematic view of this logic gate for transcription regulation is illustrated in FIG. 8. Despite the potential of CRISPR/Cas systems to efficiently generate synthetic GRN, there have been no published studies using CRISPR-based logic gates for transcription control using external stimuli in tissue engineering applications.

The AND gate described in this study has two inducible constructs encoding for a gRNA and dCas9, responding to doxycycline and climate, respectively Only in the presence of both stimuli will gRNA bind to its target DNA and recruit dCas9 to activate transcription of the target endogenous gene, BMP 2. Both constructs were cloned into transposons, as this system allows for large payloads and is usually less susceptible to epigenetic silencing¹⁷. HEK-293T cells were transfected with both constructs to create stable cell lines with a GRN to control production of endogenous BMP-2 in response to external stimuli. These engineered cells with synthetic GRN are able to produce highly regulated, localized, and bioactive BMP-2 while exhibiting a response behavior characteristic of an AND gate.

### Example 2

### Vectors

Correct assembly of the plasmids encoding for the transposons pSBtet-HH-gRNA-HDV-mCherry and pPBq-dCas9-VPR was verified by PCR colony screen, restriction analysis and DNA sequencing.

Vector pSBtet-HH-gRNA-HDV-mCherry

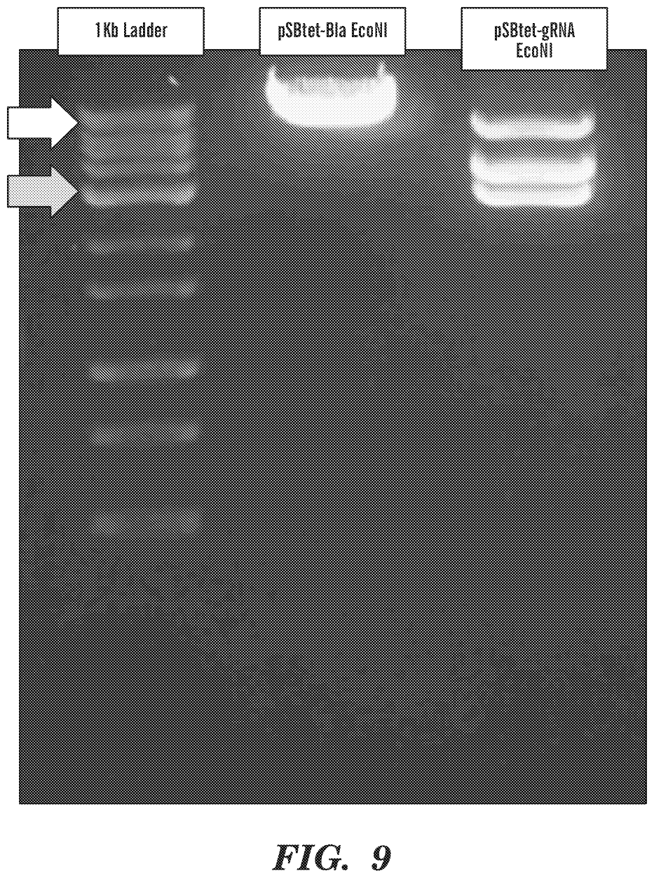

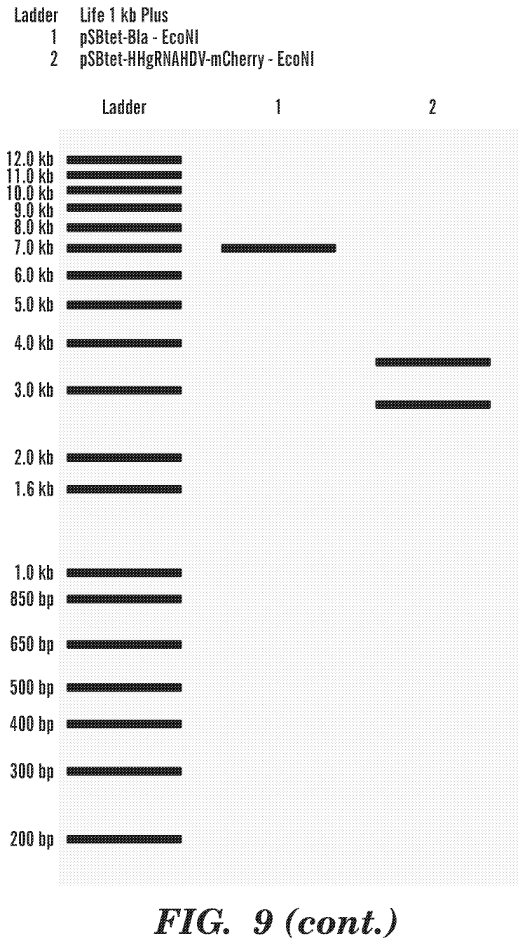

Results for restriction analysis for pSBtet-HH-gRNA-HDV-mCherry are illustrated in FIG. 9. The expected bands around 3 Kb are present in the lane corresponding to the digested vector-insert plasmid, whereas these bands are not present in the vector only plasmid. The presence of these bands indicate cloning was successful and the insert is present in the plasmid. Also present in the vector-insert lane is a third band around 7 Kb, likely corresponding to undigested plasmid.

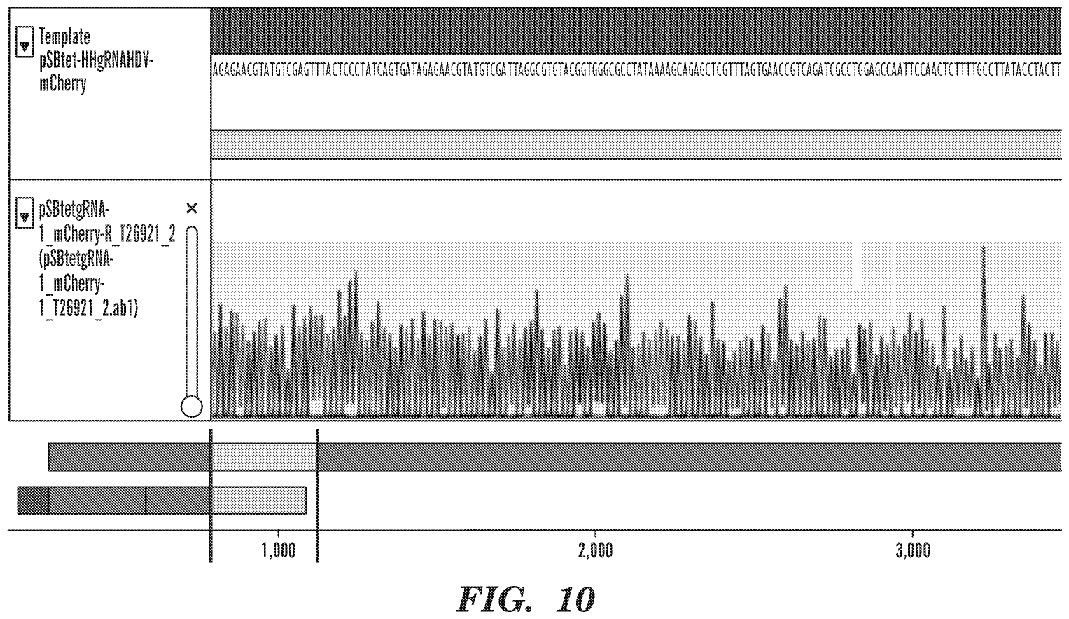

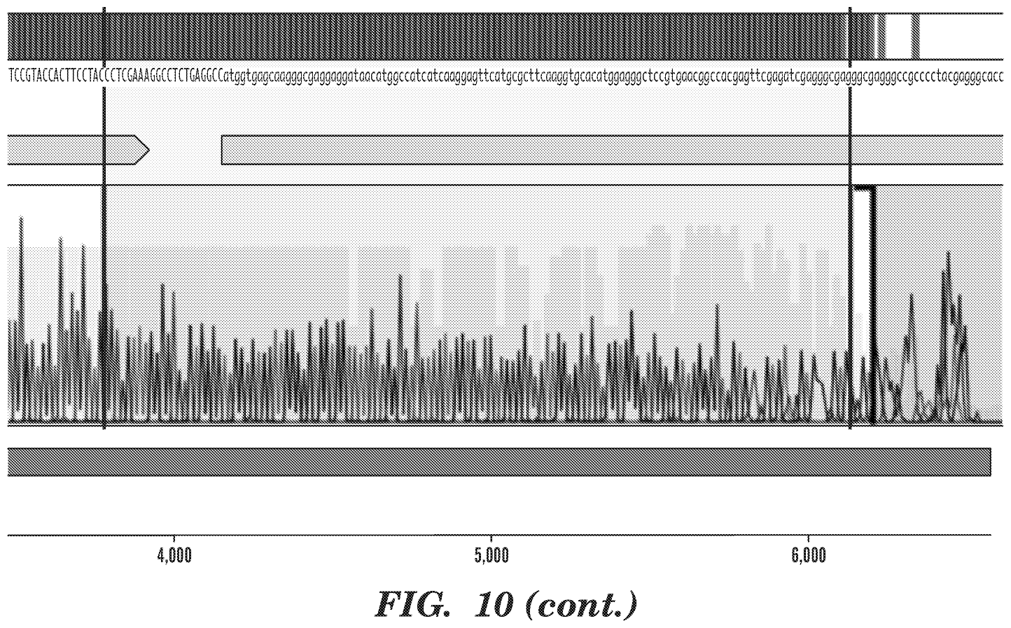

The plasmid was also sequenced by Eurofins Genomics (Louisville, Ky.), using standard reverse primers for mCherry (TTGGTCACCTTCAGCTTGG). Because mCherry is located in the insert and the direction of sequencing would generate a sequence that would cover the cloning site (SfiI recognition site) and the vector, alignment with the reference plasmid sequence would confirm insert presence and correct orientation. Sequencing results are shown in FIG. 10. The alignment suggests that the cloning was successful, resulting in correct orientation of the insert and no changes when compared to the reference sequence.

Vector pPBq-dCas9-VPR

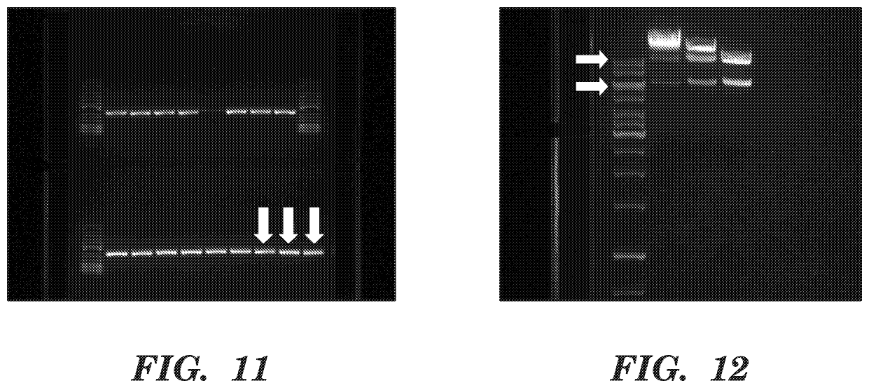

Results for the PCR colony screen for pRBq-dCas9-VPR are shown in FIG. 11. Primers targeting the dCas9-VPR insert were used to detect the presence of the plasmid in selected bacterial colonies. The results suggest that cloning and transformation were efficient and 3 colonies were selected for miniprep and downstream applications.

Results for restriction analysis for pPBq-dCas9-VPR are shown in FIG. 12. The plasmids digested with NotI and NheI show hands around 10 Kb and 6 kb, corresponding to vector and insert, respectively. These results suggest cloning was successful, the insert is present in the plasmid, and restriction enzyme recognition sites used for cloning are preserved. Colony 3 was chosen for midi prep and downstream applications.

### Input Function Tests

Function and inducibility of the plasmids encoding for the transposons pSBtet-HH-gRNA-HDV-mCherry and pPBq-dCas9-VPR was verified by FACS (for pPBq-dCas9-VPR) and fluorescence microscopy.

Vector pSBtet-HH-gRNA-HDV-mCherry

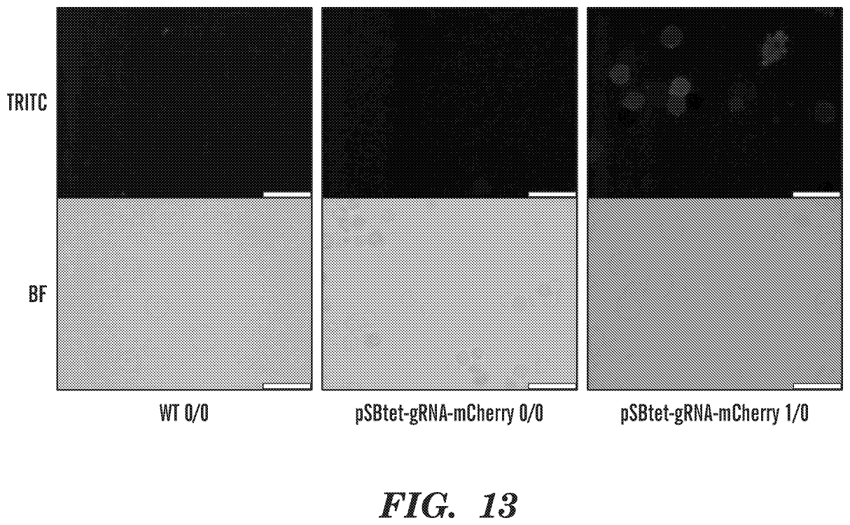

Results for fluorescence microscopy function analysis of pSBtet-HH-gRNA-HDV-mCherry are shown in FIG. 13. HEK-293T cells transfected with the inducible transposon (SB) and WT cells were imaged with a TRITC filter 24 hours after conditions (stimulated/unstimulated) were changed. WT and unstimulated SB cells show no fluorescence, while stimulated (1 μM doxycycline) SB cells display fluorescence. This expression of red fluorescent protein (mCherry) suggests that the inducible transposon is functioning correctly and implies that gRNA is being transcribed after stimulation.

Vector pPBq-dCas9-VPR

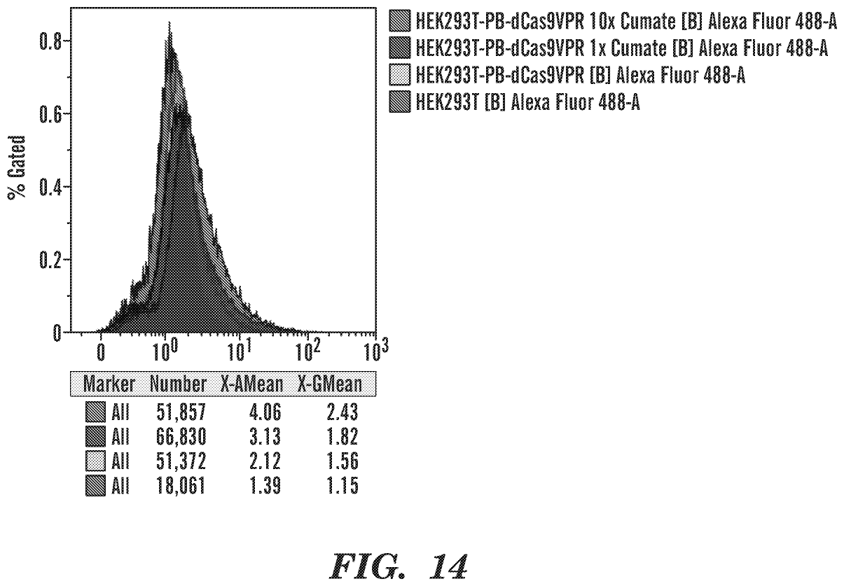

Results for FACS of pPBq-dCas9-VPR are shown in FIG. 14. HEK-293T cells transfected with the inducible transposon (PB) and WT cells were sorted 24 hours after conditions (stimulated/unstimulated) were changed. The right shift in the x axis suggests that PB cells express EGFP in a titratable fashion after cu mate stimulation and implies that dCas9-VPR is co-expressed after stimulation.

Vectors pSBtet-HH-gRNA-HDV-mCherry and pPBq-dCas9-VPR Co-Expression

Results for fluorescence microscopy function analysis of pSBtet-HH-gRNA-HDV-mCherry and pPBq-dCas9-VPR are shown in FIG. 15. HEK-293T cells transfected with both inducible transposons (SB/PB) and WT cells were imaged with TRITC and GFP filters 24 hours after conditions (stimulated/unstimulated) were changed. WI and unstimulated SB/PB cells show no fluorescence, while stimulated (1 μM doxycycline and 300 μg/mL cumate) SB/PB cells display fluorescence. This co-expression of red (mCherry) and green (EGFP) fluorescent proteins suggests that both inducible transposons can function correctly while present concurrently in the same cell, implying that dCas9-VPR and gRNA are present in SB/PB cells after stimulation.

### AND Gate Function Tests

### Luciferase Reporter Assay

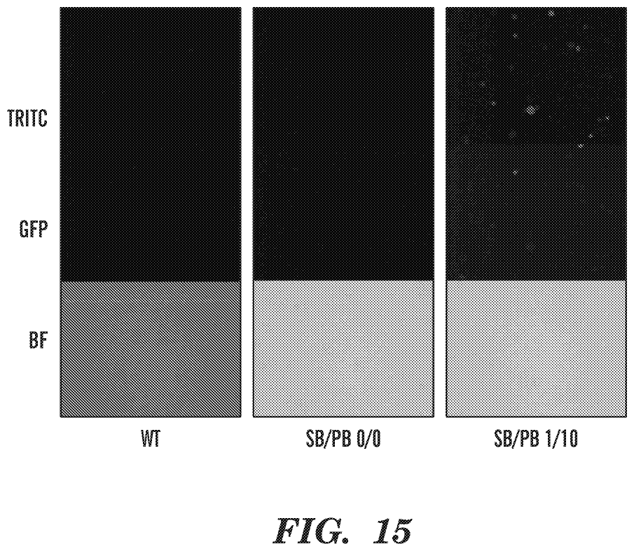

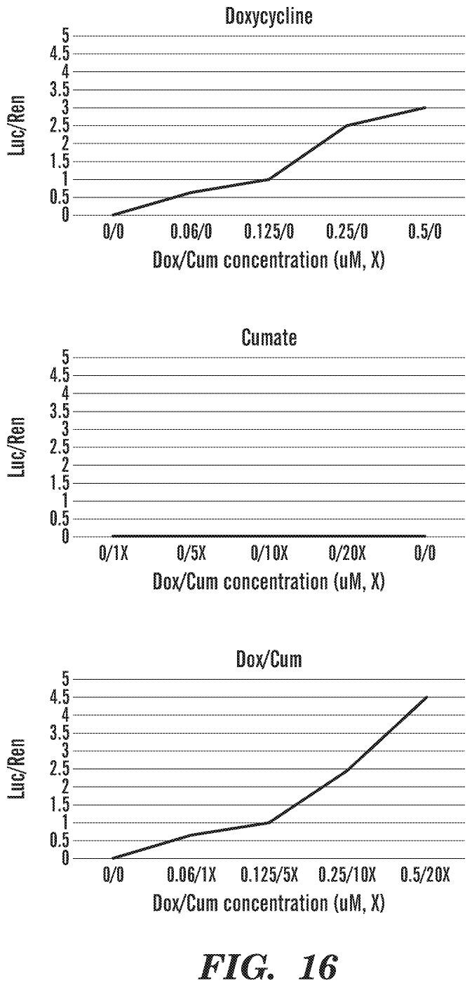

Results for luciferase reporter assay of synthetic promoter reporter version of AND gate cells are shown in FIG. 16. SB/PB cells were transfected with the P1 reporter and Renilla vectors, then assayed for luciferase activity 48 hours after conditions (stimulated/unstimulated) were changed. SB/PB cell groups without stimulation, or curate stimulation have similar luminescence as WT control groups (0/0, 1/0, 0/10, and 1/10). SB/PB cell groups with doxycycline and doxycycline/cumate stimulation show a significant increase in luciferase activity.

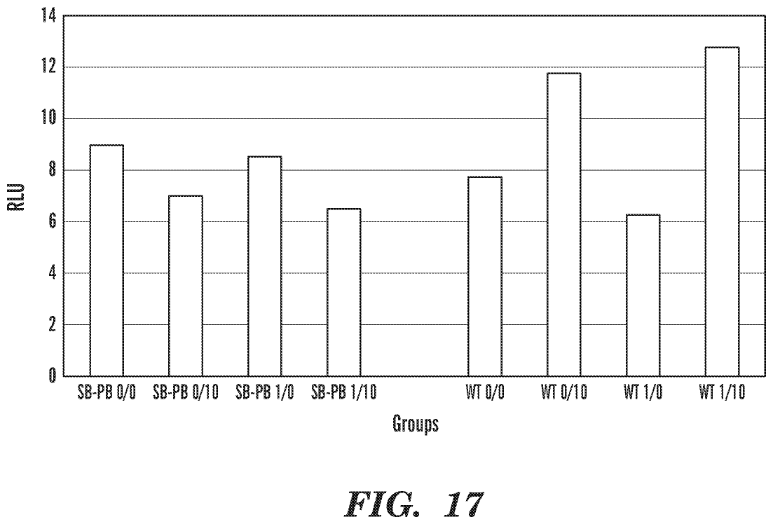

Results for luciferase reporter assay of BMP-2 promoter reporter version of AND gate cells are shown in FIG. 17. SB/PB cells were transfected with the BMP-2 goClone reporter vector and assayed for luciferase activity 48 hours after conditions (stimulated/unstimulated) were changed. All SB/PB cell groups (0/0, 1/0, 0/10, and 1/10) have similar luminescence as WT control groups (0/0, 1/0, 0/10, and 1/10). There were no groups with significant increase in luciferase activity, suggesting a lack of transcription activation of the Renilla gene controlled by the dCas9-VPR/gRNA complex binding to the BMP-2 promoter in the GoClone reporter.

Altogether the results of the synthetic promoter reporter plasmid version of the AND gate suggest the presence of cryptic tetracycline-responsive elements (TREs) in the reporter plasmid. These TRE- would bind the rtTA present from the pSB-TRE-gRNA1-Bla construct and express luciferase. The activation of luciferase in the presence of doxycycline stimulation alone would mask AND gate behavior, if present, and suggests the need to design a different reporter. The observed truth table is illustrated in Table 1, referencing the behavior exhibited in FIG. 16.

TABLE 1
Observed Truth table for synthetic promoter
reporter version of AND gate

| Dox | Cum | Luc |
|---|---|---|
| 0 | 0 | 0 |
| 1 | 0 | 1 |
| 0 | 1 | 0 |
| 1 | 1 | 1 |

TABLE 2
Observed Truth table for BMP-2 promoter
reporter version of AND gate

| Dox | Cum | Luc |
|---|---|---|
| 0 | 0 | 0 |
| 1 | 0 | 0 |
| 0 | 1 | 0 |
| 1 | 1 | 0 |

The results of the BMP-2 promoter reporter plasmid version of the AND gate indicate that there are no responsive elements in the reporter plasmid that would react to the stimuli and mask AND gate behavior. Nonetheless, luciferase activity was not observed in any of the groups, shown in Table 2 (referencing the behavior exhibited in FIG. 17), suggesting a lack of transcription activation by the dCas9-VPR/gRNA complex. The results from the plasmid verification tests indicate correct cloning of the input vectors, while the input plasmid function tests suggest that both input transposons have been integrated into the genomic DNA of the SB/PB cells and are inducible. The presence of fluorescent markers is indicative of gene expression driven by the stimuli, but it is not a direct measurement of the product of interest. Because of the large size of the dCas9-VPR protein it is possible that the luciferase readout needs to be postponed to 72-96 hours after stimulation. The Renilla gene in the goClone reporter has been engineered with a protein destabilization domain (from the mouse Ornithine Decarboxylase), so there should be little concern of protein accumulation that would mask differences of expression profiles. The necessity of dCas9-VPR translation and further trafficking into the nucleus mediated by the simian virus 40 (SV40) nuclear localization signals (NLS) present in the protein, make correct reading frame concordance of all elements paramount, and incorrect translation would prevent binding to target DNA via gRNA interaction and transcription activation of BMP-2 goClone Renilla reporter. The transcription of gRNA, ribozyme cleavage, and nuclear localization is more predictable and less likely to be the cause of lack of transcription activation. On the other hand, gRNA targeting for transcription activation is still in its infancy and there are no validated gRNAs for BMP-2 transcription activation. The in silico design of gRNAs used in this study was an attempt to circumvent typical gRNA screen procedures used for other CRISPR/Cas9 genomic engineering applications. It is possible that despite careful consideration of the highly-conserved sequences in the BMP-2 promoter region and in silico design of gRNA targeting these sites less than 100 bp upstream of the TSS, that the gRNA designed for the BMP-2 promoter reporter plasmid version of the AND gate is not effectively inducing activation and expression of the Renilla reporter.

### Next Steps

Despite the lack of luciferase activity evidenced in the BMP-2 promoter reporter plasmid version of the AND gate, there is a possibility that endogenous BMP-2 transcription is being activated. A simple dot blot assay where polyclonal antibodies for human BMP-2 will be used to detect presence of BMP-2 in SB/PB cell culture supernatant will be performed. SB/PB cells will be cultured in four conditions (unstimulated, doxycycline, climate, and doxycycline/cumate stimulated), and a dot blot performed 72 hours after stimulation. The results will be compared to a standard curve of known concentrations of rhBMP-2.

The lack of transcription activation described for the BMP-2 promoter reporter plasmid version of the AND gate has two possible causes, as discussed above: lack of proper dCas9-VPR function, or ineffective targeting of the gRNA. By designing a gRNA screen experiment using constitutively expressed dCas9-VPR and gRNA to drive BMP-2 promoter-driven Renilla, the effectiveness of either input plasmid can be elucidated. By using a constitutively expressed dCas9-VPR that has been successfully used to drive gene expression in other endogenous genes (Church Lab), the variable of cloning-induced problems is circumvented. The gRNA screen would include the gRNA designed for the BMP-2 promoter reporter plasmid version of the AND gate, along with 9 other candidate gRNA sequences targeting different areas of the BMP-2 promoter region. Design of gRNA sequences follows a structured approach, where sequences that are adjacent to a PAM are selected after analysis to determine the likelihood of targeting other genes, DNA binding effectiveness, and thermodynamic stability of the resulting gRNA. The number of possible gRNA sequences in the 300 bp upstream of the TSS of BMP-2 is therefore limited, informing the choice of using 10 total gRNA candidates for the screen. HEK 293T WT cells will be co-transfected with the dCas9-VPR, gRNA, and BMP-2 goClone plasmids and luciferase activity measured 48 and 72 hours after transfection. If activation of the BMP-2 promoter driven Renilla gene is detected in the cells transfected with the gRNA designed for the BMP-2 promoter reporter plasmid version of the AND gate, this will suggest problems with the pPBq-dCas9-VPR transposon and would imply the need to repeat the cloning of the dCas9-VPR inducible transposon. If there is no luciferase activity with the gRNA designed for the BMP-2 promoter reporter plasmid version of the AND gate, but there is luciferase activity detected with one or more of the other candidate gRNA sequences, this would evince the ineffective targeting of the BMP-2 promoter for activation by the gRNA and the need to clone the best performing candidate into the pSBtet-HH-gRNA-HDV-mCherry plasmid.

Once correctly functioning input vectors have been generated by the steps detailed above, luciferase activity is assessed to test AND gate function. Once an observed truth table from the luciferase assays matches the expected truth table, corroborating proper AND gate behavior, the transcription activation of endogenous BMP-2 will be confirmed with dot blot assay. Subsequently, the bioactivity of the AND gate-controlled BMP-2 will be assessed. W20-17 murine cells, an osteosarcoma cell line known for a linear increase in ALP activity in response to BMP-2, will be cultured in conditioned media. The conditioned media will be the supernatants of the SB/PB cells in unstimulated and stimulated conditions. Increase in alkaline phosphatase (ALP) activity in the W20-17 cells exposed to doxycycline and comate stimulated SB/PB cell medium would confirm bioactivity of the AND gate-controlled BMP-2. Furthermore, Human Adipose-derived Stem Cells (hASCs) will be cultured in the same conditions as the W20-1.7 cells, and their differentiation in response to AND gate-controlled BMP-2 will be assessed. Morphological changes, qPCR with primers for osteogenic markers (Osteocalcin, Osteopontin, etc), and Alizarin. Red S staining (to assay mineralization) will be used to determine the potential of AND gate-controlled BMP-2 to drive hASC differentiation towards osteoblastic lineages.

Once proper AND gate behavior is exhibited and bioactivity of AND gate-controlled BMP-2 is confirmed, the system is used to generate engineered hASCs. Transfection protocols will be optimized to rapidly generate stable hASC cell lines with both input transposons integrated in their genomic DNA. The use of these engineered hASCs will be preferable for potential clinical applications due to their relative abundance (for a stem cell type), potential to differentiate into osteoblastic lineages, and ability, once engineered with the AND gate, to secrete endogenously-produced BMP-2 in response to external stimuli.

Another possible avenue of research would be the ability to target the integration of the AND gate components to safe harbor loci in the human genome. Safe harbor loci are sequences that have been identified as lacking function and are generally regarded as targets where integration has virtually no potential for deleterious effects like frame-shift mutations, activation of oncogenes, etc. Use of these safe harbor loci as targets for AND gate components would mitigate concerns of possible negative side effects caused by random integration of DNA.

Furthermore, the capability of multiplexing gRNA sequences in the pSBtet-HH-gRNA-HDV-mCherry transposon targeting different genes for transcription activation could be explored. This strategy would allow exploration of bone tissue engineering using multiple growth factors, tackling the limitations of large defect regeneration. One example of a limitation frequently encountered is lack of angiogenesis limiting the size of a tissue engineered graft due to oxygen and nutrient delivery, and waste removal, restrictions. Vascular Endothelial Growth Factor (VEGF) has been identified as an important stimulator of angiogenesis¹⁸. The effect of endogenously-produced, AND gate-controlled BMP-2 and VEGF secreted concomitantly in the differentiation potential of hASCs into osteoblastic lineages, and osteoinductive properties of tissue engineered bone grafts could be explored.

Ultimately, the incorporation of CRISPR-based logic gates and synthetic biology approaches into the tissue engineering arsenal are promising tools for the control of the cell milieu and stem cell differentiation. The AND gate-controlled, localized delivery of BMP-2 would be the first application of CRISPR-based logic gates in tissue engineering. The present study is a proof of concept for a basic circuit that would serve as a starting point for the development of more complex circuits that include multiple logic gates layered orthogonally, multiple targets, different physico-chemical stimuli, etc. Future avenues of research will be evolving informed by discoveries made possible by the ability to tightly regulate expression of these intricate signaling cascades, launching a new area of study for the already interdisciplinary field of tissue engineering.

### Vectors

### Synthetic Promoter Reporter Version

A logical AND gate was designed using inducible transposon dCas9 and gRNA constructs as inputs, and a synthetic promoter-luciferase reporter vector as output. The transposon system was chosen as it allows for large payloads and is usually less susceptible to epigenetic silencing when correctly integrated. The inputs are as follows: pSB-TRE gRNA1-Bla, a doxycycline-inducible Sleeping Beauty (SB) transposon with a blasticidin selection marker and a gRNA cloned into its multi-cloning site (MCS). The RNA used is flanked by a Hammerhead ribozyme (HH) on the 5′ end and a Hepatitis Delta Virus ribozyme (HDV) on the 3′ end. These self-cleaving ribozymes allow the use of a PolII promoter and a gRNA that does not start with a C nucleotide. The SB vector was a gift from Eric Kowarz (Goethe University, Germany, Addgene plasmid #60510) and the HH-gRNA-HDV construct was amplified by PCR from a plasmid provided by Timothy Lu (Massachusetts Institute of Technology, Massachusetts, Addgene #55200). The second input is pPBq-dCas9-VPR, a cumate-inducible PiggyBac (PB) transposon from System Biosciences (Palo Alto, Calif., PBQM812A-1) with a puromycin selection marker and a dCas9-VPR cloned into its MCS. The dCas9 was PCR amplified from a plasmid provided by George Church (Harvard University, Massachusetts, Addgene #63798) and cloned into the PB vector with TOPO cloning. The nucleolytically-inactivated Cas9 has a VP64-p65-Rta. (VPR) fused to its C terminus, which acts as a transcription activator when the dCas9 binds to the gRNA. The reporter plasmid P1-Luc, is a pGL4.14-Luc2 (Promega, Madison, Wis.) backbone vector with a synthetic promoter (P1) cloned into its MCS, expressing firefly luciferase. The P1 promoter was PCR amplified from a plasmid provided by Timothy Lu (Massachusetts Institute of Technology, Massachusetts, Addgene #5519).

When the cells transfected with all three plasmids are exposed to doxycycline (Toed s Bioscience, Bristol, United Kingdom), and cumate (System Biosciences, Palo Alto, Calif.), the dCas9 and gRNA genes are expressed, binding to the P1 promoter site, creating an activator complex that runs expression of firefly luciferase. The luciferase reporter was chosen for its ease of use and sensitivity. The expected truth table, showing the on-off conditions depending on inputs exemplary of an AND gate, is shown in Table 3.

TABLE 3
Expected truth table for CRISPR AND gate.

| Dox | Cum | Luc |
|---|---|---|
| 0 | 0 | 0 |
| 1 | 0 | 0 |
| 0 | 1 | 0 |
| 1 | 1 | 1 |

### BAP-2 Promoter Reporter Version

The curate-inducible PiggyBac transposon vector, pPBq-dCas9NPR, from the previous version was used as an input for this version. The second input was a doxycycline-inducible Sleeping Beauty transposon with a gRNA targeting a site <100 base pairs upstream of the transcription start site (TSS) of human BMP-2 cloned into its MCS. The gRNA was flanked by HH and HDV ribozymes, as well as mCherry upstream of the HH ribozyme. The mCherry-HH-gRNA-HDV assembly was flanked with MIT sites and synthesized by Integrated DNA Technologies (Coralville, Iowa) as a double-stranded DNA gBlock. Restriction enzyme cloning, with SifI (Themo Scientific, Waltham, Mass.), was used to insert this assembly into the SB vector, generating the input vector pSBtet-HH-gRNA-HDV-mCherry. The reporter vector is a Switchgear Genomics goClone reporter (S710234, SwitchGear Genomics, Menlo Park, Calif.). This is a ready to use plasmid that includes a BMP-2 promoter and a Renilla gene, a marine luciferase.

When the cells transfected with all three plasmids are exposed to doxycycline and cumate, the dCas9 and gRNA genes are expressed, binding to the BMP-2 promoter site, creating an activator complex that runs expression of Renilla and BMP-2.

### Plasmid Verification

All plasmids were purified with GeneJet miniprep kits (Thermo Scientific, Waltham, Mass.) following manufacturer's protocol.

pSBtet-HH-gRNA-HDV-mCherry was verified with restriction analysis (RA), using EcoNI (Thermo Scientific, Waltham, Mass.), and plasmids were digested for 15 minutes before gel electrophoresis (1.5% agarose). This plasmid has an EcoNI restriction site in the vector and one in the insert, so RA should reveal two fragments of approximately 3 kb if cloned correctly. The selected colonies were also sequenced to verify insert presence and correct orientation.

PB-Q-dCas9-VPR-Puro was verified with RA, using NotI (Thermo Scientific, Waltham, Mass.) and NheI (Thermo Scientific, Waltham, Mass.). Bands were expected at 10 and 6 kb, for vector and insert, respectively. Plasmids were digested for 15 minutes before gel electrophoresis (1% agarose). Bacterial colonies were also PCR screened, using primers targeted at the insert. Briefly, colonies were selected and marked, bacteria were mixed with PCR master mix (Phusion High Fidelity, New England Biolabs, Ipswich, Mass.), PCR program was 98 C for 30 seconds before running 30 cycles, then gel electrophoresis for PCR products (1.5% agarose).

### Transfections

FEK-293T cells (American Type Culture Collection, Manassas, Va.) were transfected with pSB-TRE-gRNA1-Bla, or pSBtet-HH-gRNA-HDV-mCherry, using X-tremeGENE 9 (Roche, Basel, Switzerland) following manufacturer's protocol. Cells were selected 72 hours post-transfection with blasticidin (Tocris Bioscience, Bristol, United Kingdom) at a concentration of 5 μg/ml for 5 days, generating a stable cell line labeled SB cells. SB cells were then transfected with pPBq-dCas9-VPR, and selected with a combination of blasticidin and puromycin. (Tocris Bioscience, Bristol, United Kingdom) at concentrations of 5 μg/ml and 3 μg/ml, respectively, for 3 days, generating a stable cell line labeled SB/PB cells.

### Input Function Tests

To test the inducibility of dCas9 by comate, the presence of HOT in the PB vector was exploited and the cells exposed to varying concentrations of climate were sorted by Fluorescence-activated cell sorting (FACS). Wild type (WT) HEK-293T cells were used as a technical control (background) and as a reference point to determine leakiness of the system when untreated. WT and HEK 293T cells transfected with PB-Q-dCas9-VPR-Puro were seeded in 6 well plates (2.0×10⁵ cells/well) and stimulated with curate (0×, 1× (30 μg/ml) and 10× (300 μg/ml) solutions). 24 hours later, the cells were sorted by FACS with an Alexa 488 filter.

pPBq-dCas9-VPR construct was also targeted to endogenous genes (Interferon-induced protein with tetratricopeptide repeats 1 (IFIT1) and BMP-2). The choice of target was influenced by the presence of previously designed gRNAs for IFTIT1 and BMP-2 as a gene of interest. The activation of IFIT1 was assayed by qPCR. (Results not shown).

To test the functionality of pSBtet-HH-gRNA-HDV-mCherry, inducibility of gRNA by doxycycline was assayed, exploiting the presence of mCherry. WT HEK-293T cells were used as a technical control. WT and pSBtet-HH-gRNA-HDV-mCherry transfected cells were seeded in a 24 well plate (0.5×10⁶ cells/well) and stimulated with doxycycline (0 and 1 μM solutions), 24 hours later, cells were imaged using an Olympus microscope (Model BX43F) equipped with Olympus DP73 digital camera and fluorescent light source (mCherry excited at 587 nm), 20× magnification, 250 ms exposure, with a bright field and TRIT-C filter. Representative images were used for analysis.

To test the functionality of the pSB-TRE-gRNA1-Bla vector, a luciferase assay using a constitutively-expressed dCas9VPR and the P1-Luc construct was performed. HEK 293T cells transfected with the pSB-TRE-gRNA1-Bla construct were plated in 24 well plates (5×10⁴ cells/well). The cells were transfected with dCas9-VPR plasmid (transient, constitutive expression), P1-Luc and Renilla vectors using X-tremeGENE 9 following manufacturer's protocol. WT cells and cells without the dCas9-VPR plasmid were used as controls. Cells were stimulated with varying doses of doxycycline (0, 0.25, 0.5 and 1 μM). Readout by LightSwitch Dual Assay kit (DA010, SwitchGear Genomics, Menlo Park, Calif.) was performed 48 hours post-stimulation. Luciferase Relative Light Units (RLU) were normalized to Renilla.

To test the functionality of pSBtet-HH-gRNA-HDV-mCherry and pPBq-dCas9-VPR co-expressed in a cell, inducibility of gRNA by doxycycline and dCas9-VPR was assayed, exploiting the presence of mCherry and EGFP, respectively. WT HEK-293T cells were used as a technical control. WT and pSBtet-HH-gRNA-HDV-mCherry/pPBq-dCas9-VPR transfected cells were seeded in a 24 well plate (0.5×10⁶ cells/well) and stimulated with doxycycline and climate (0/0 and 1 μM/10× solutions). 24 hours later, cells were imaged using an Olympus microscope Model BX43F) equipped with Olympus DP73 digital camera and fluorescent light source (mCherry excited at 587 nm, EGFP at 488 nm), 20× magnification, 250 ms exposure, with a bright field. GFP, and TRIT-C filter. Representative images were used for analysis.

### AND Gate Function Tests

The function of the synthetic promoter reporter version of the AND gate circuit was assessed using a LightSwitch Dual Assay kit. Cells transfected with pPBq-dCas9-VPR and pSB-TRE-gRNA1-Bla were selected with puromycin and blasticidin. These cells were then seeded on 24 well plates (5×10⁴ cells/well). The cells were then transfected with P1-Luc and Renilla vectors using X-tremeGENE 9 following manufacturer's protocol, and stimulated with doxycycline and curate to create 4 conditions (untreated, doxycycline (1 μM), cumate (10×) and doxycycline/cumate (1 μm/10×)). Luciferase and Renilla were read 48 hours post-stimulation (SpectraMax L, Molecular Devices, Sunnyvale, Calif.) measuring luminescence of all wavelengths, 2 seconds per well.

The function of the BMP-2 promoter reporter version of AND gate circuit was assessed using a LightSwitch Luciferase Assay kit (LS010, SwitchGear Genomics, Menlo Park, Calif.). Cells transfected with pPBq-dCas9-VPR and pSBtet-HH-gRNA-HDV mCherry were selected with puromycin and blasticidin. These cells were then seeded on 96 well opaque white plates (5×10⁴ cells/well). The cells were then transfected with BMP-2 goClone reporter vector using X-tremeGENE 9 following manufacturer's protocol, and stimulated with doxycycline and curate to create 4 conditions (untreated, doxycycline (1 μM), cumate (10×) and doxycycline/cumate (1 μm/10×)). Luciferase activity was read 48 hours post-stimulation (SpectraMax i3, Molecular Devices, Sunnyvale, Calif.) measuring luminescence of all wavelengths, 2 seconds per well.

### Example 3

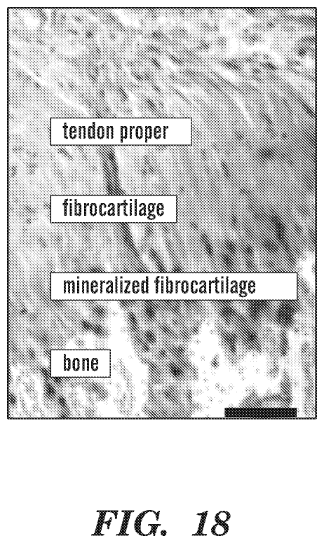

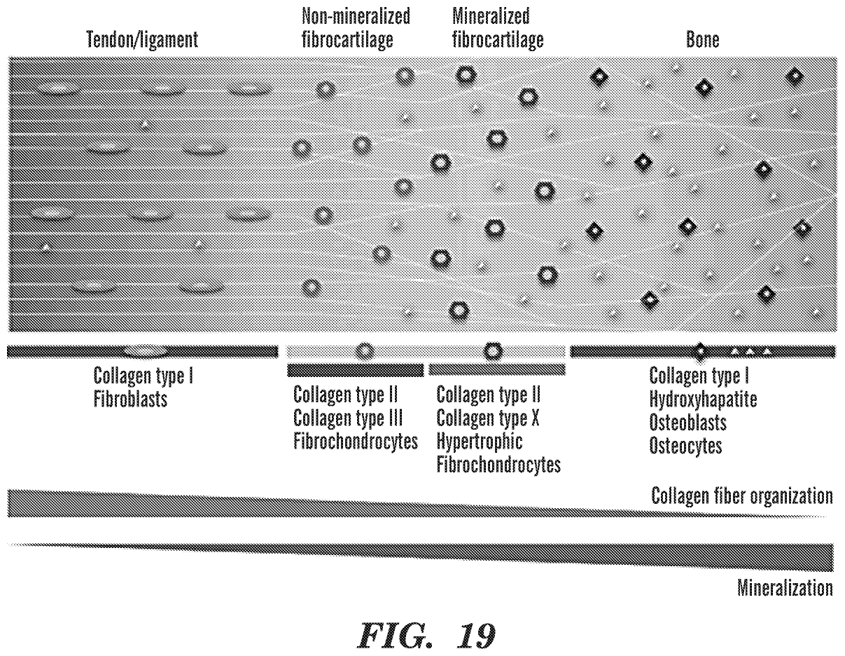

In the US alone, more than half of the 100,000 people that undergo anterior cruciate ligament reconstruction surgery and nearly 80 percent of the 500,000 that have rotator cuff repairs every year will experience repair failure, mostly due to the current inability to regenerate a properly functioning enthesis¹⁻⁴. High failure rates lead to significantly increased cost and time to recovery¹. The specialized interface referred to as the enthesis, the insertion of tendons/ligaments into bone, is a highly-organized tissue presenting continuous gradients of structural and mechanical properties that allow smooth force transfer, protecting and maintaining the tendon/ligament insertion. The lack of regeneration of the enthesis leads to high failure rates after these procedures due to formation of a discrete transition of tissue characteristics which acts as a force concentrator that, under mechanical loading, is the site of rupture^{1, 4}. The clinical importance of regenerating the natural structure of entheses have made them a recent focus of tissue engineering, with the generation of smooth mechanical and structural gradients still a major challenge. The enthesis is divided into four zones with extracellular matrices (ECM) and cell types varying in a smooth gradient fashion (FIGS. 18 and 19)¹. The first zone of the enthesis is the tendon/ligament proper, characterized by parallel collagen type I fibers, arrays of elongated fibroblasts, and proteoglycans². This zone has mechanical properties similar to those of the tendon/ligamene³. The second zone is uncalcified fibrocartilage with round fibrochondrocytes arranged in rows surrounded by ECM composed of aggrecan and collagen types I, II, and III¹. This zone is avascular and functions as a force damper, dissipating stress generated by bending collagen fibers in the tendon¹. The third zone is avascular, calcified fibrocartilage with hypertrophic fibrochondrocytes and ECM composed of aggrecan and collagen types II and X^{1, 2}. The third zone is at the boundary with subchondral bone and is highly irregular, providing mechanical integrity through the attachment of the mineralized layer to the bone¹. Some studies suggest that this zone is important for blocking blood supply from reaching the avascular zones in the enthesis and tendon/ligament, preventing communication between the compartments¹. The fourth zone is the bone proper, characterized by populations of osteoblasts, osteoclasts, and osteocytes residing in a disorganized ECM of type I collagen and hydroxyapatite¹.

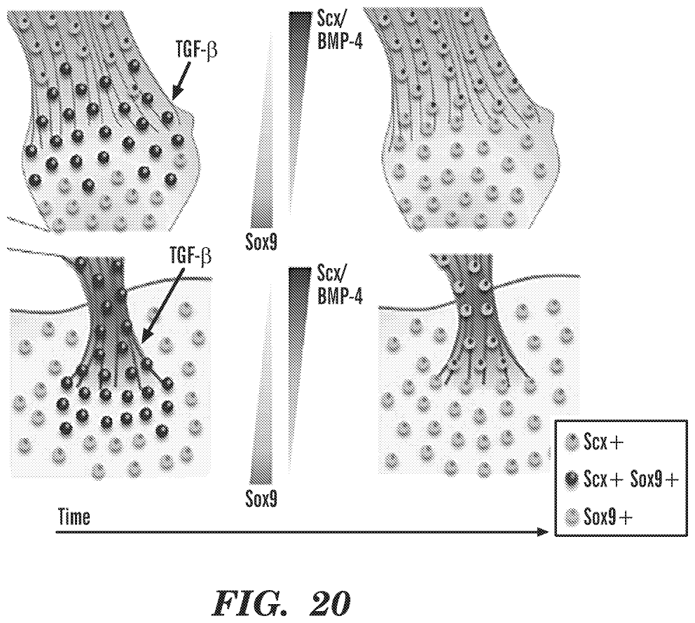

Though the four zones exhibit clear compositional and functional differences, it needs to be stressed that the structure is continuous and the gradient contributes greatly to the function of the tissue¹⁻⁴. The development of these zones occurs postnatally, although the exact process is poorly understood. A combination of physicochemical stimuli drive the gradient formation in the interfacial structure²⁻⁴. Transcription factors Sox9 and Scleraxis (Scx) play important roles in the development of the enthesis, and are arranged in countergradient fashion in immature postnatal entheses (FIG. 20)². Scx is associated with tenogenesis, while Sox9 is widely accepted to drive chondrogenesis. The primitive enthesis is composed of a pool of progenitor cells that express both transcription factors (Scx+/Sox9+), and progressively polarizes to include a pool of Scx−/Sox9+ and Scx+/Sox9− cells^{2, 4}. The development of the Scx and Sox9 countergradient is believed to be stimulated by transforming growth factor-β(TGF-β) as well as mechanical forces acting on the enthesis³. These stimuli are both present during healing responses, but it is not yet understood why the result is fibrous scar tissue without the functional structure of the enthesis^{2,4}. Several studies have focused on the use of other growth factors for enthesis regeneration, with platelet-derived growth factor (PDGF-BB) and bone morphogenetic growth factor-2 (BMP-2) garnering great attention^{5,9}. PDGF-BB has been associated with ligament/tendon regeneration both in vitro and in vivo⁵⁻⁷. BMP-2 has been widely used for bone regeneration^{5, 8, 9}. Both growth factors have been FDA approved for clinical use, and in a preclinical study where a gradient of BMP-2 and PDGF-BB was immobilized in a porous membrane, they directed mesenchymal stem cell (MSC) differentiation in vitro to recreate gradient structures⁵. While this study is promising, the use of recombinant proteins presents several challenges: high cost, handling difficulty, supraphysiological dosages, and less effectiveness than paracrine, endogenous stimulation. The adult enthesis has no native populations of stem cells in the tendon/ligament portion, but the presence of osteoblastic precursors is evident in the bone zone⁴. This lack of stem cells and the poor vascularization of the enthesis zones 1-3 partially explain the lack of proper healing³. Most current studies that focus on cell-based strategies for enthesis regeneration use MSCs (bone marrow and adipose-derived) due to their relative abundance, ease of expansion, and ability to differentiate into the cells that populate the native enthesis¹⁻⁴.

Clustered regularly interspaced short palindromic repeats (CRISPR) systems are nucleic acid-targeting, RNA-directed adaptive restriction machineries used by prokaryotes as defense mechanisms against mobile genetic elements¹⁰. Although the adaptation of these systems to genome editing has revolutionized genetic engineering, the technology has been used for other applications, including live-cell DNA imaging and transcriptional activation/repression¹¹⁻¹³. CRISPR activation/repression is a simple approach to target gene expression control in the mammalian genome¹¹. The use of guide RNA (gRNA)-guided nucleolytically-inactive CRISPR-associated proteins (dCas9 and, in our study, dCpf1) fused to transcriptional regulators is an efficient and cost-effective solution to targeting and regulating single or multiple genes¹³. This system improves on the targeting efficiency of RNA-interference approaches, and is simpler, less costly, and more readily deliverable into cells than protein-based tools like zinc finger and transcription-activator-like effector nucleases^{11, 13}. In CRISPR activation/repression, a gRNA binds target DNA sequences, dictated by a designed 20 nucleotide complementary sequence adjacent to a protospacer-adjacent motif (PAM)¹⁰. The gRNA localizes and complexes a dCas9 protein that activates/represses gene expression¹⁰.

As a proof of concept for component 2, a logical AND gate was designed using inducible transposon dCas9 and gRNA constructs as inputs and a synthetic promoter-luciferase reporter vector as output. The transposon system was chosen as it allows for large payloads and is usually less susceptible to epigenetic silencing. The first input is pSB-TRE-gRNA1-Bla, a doxycycline-inducible Sleeping Beauty (SB) transposon, a gift from Eric Kowarz (Addgene plasmid #60510), with an HH-gRNA-HDV amplified by PCR from a plasmid provided by Timothy Lu (Addgene #55200) cloned into its multiple cloning site (MCS). The second input is PB-Q-dCas9-VPR-Puro, a cumate-inducible PiggyBac (PB) transposon from SBI (PBQM812A-1) with a dCas9-VPR PCR-amplified from a plasmid provided by George Church (Addgene #63798) cloned into its MCS. The nucleolytically-inactivated Cas9 has a VP64-p65-Rta (VPR) fused to its C terminus, which acts as a transcription activator. The reporter plasmid P1-Luc, is a pGL4.14-Luc2 (Promega) backbone vector with a synthetic promoter (P1) PCR-amplified from a plasmid provided by Timothy Lu (Addgene #55197) cloned into its MCS.

HEK-293T cells were selected for the preliminary studies due to their ease of culture and transfection. When HEK-293T cells transfected with all three plasmids are exposed to doxycycline and cumate, the dCas9 and gRNA genes are expressed, binding to P1, creating an activator complex that drives expression of firefly luciferase (Table 4). The correct cloning of the plasmids was verified by restriction analysis and DNA sequencing.

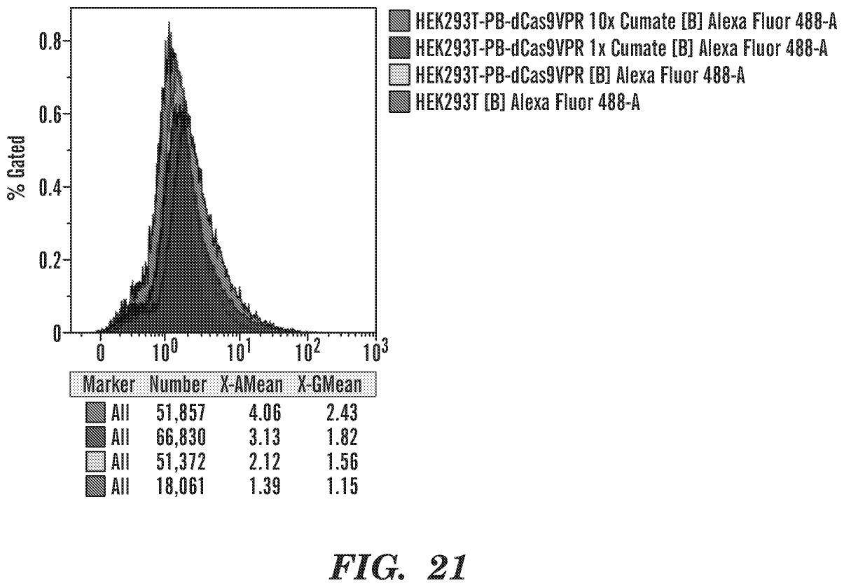

To test the inducibility of dCas9 by cumate, the presence of green fluorescent protein (GFP) in the PB vector was exploited and the cells exposed to varying concentrations of cumate were sorted by FACS. Wild type (WT) HEK-293T cells were used as a technical control (background) and as a reference point to determine leakiness of the system. WT and HEK-293T-PB-Q-dCas9-VPR-Puro cells were seeded in 6-well plates (2.0×10⁵ cells/well) and stimulated with cumate (0×, 1×, and 10× solutions) and sorted by FACS with an Alexa 488 filter 24 h later (FIG. 21). The results indicate slight leakiness and that the induction of dCas9 by cumate is titratable. The level of activation evidenced is expected and comparable to previous experiments using cumate-inducible and constitutively-expressed PB vectors conducted by the Espinosa group at Karolinska Institutet in Stockholm, Sweden.

TABLE 4
Expected truth table for CRISPR AND gate.

| Dox | Cum | Luc |
|---|---|---|
| 0 | 0 | 0 |
| 1 | 0 | 0 |
| 0 | 1 | 0 |
| 1 | 1 | 1 |

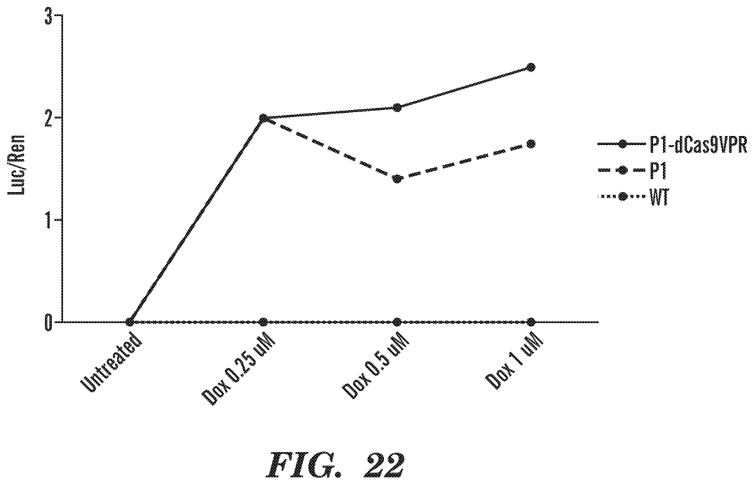

To validate pSB-TRE-gRNA1-Bla, a luciferase assay using constitutively-expressed dCas9-VPR and P1-Luc was performed. HEK-293T-pSB-TRE-gRNA1-Bla cells were plated in 24 well plates (50,000 cells/well) and transfected with dCas9-VPR, P1-Luc, and Renilla vectors. WT HEK-293T and cells without dCas9-VPR were used as controls. Doxycycline stimulation (0, 0.25, 0.5, and 1 μM) was followed by dualGlo assay 48 h later (FIG. 22).

Luciferase values were normalized to Renilla. Activation of luciferase in the absence of dCas9-VPR led us to suspect the presence of cryptic tetracycline responsive elements (TRE) in P1-Luc that bind reverse tetracycline-controlled transactivators (rtTA) from pSB-TRE-gRNA1-Bla.

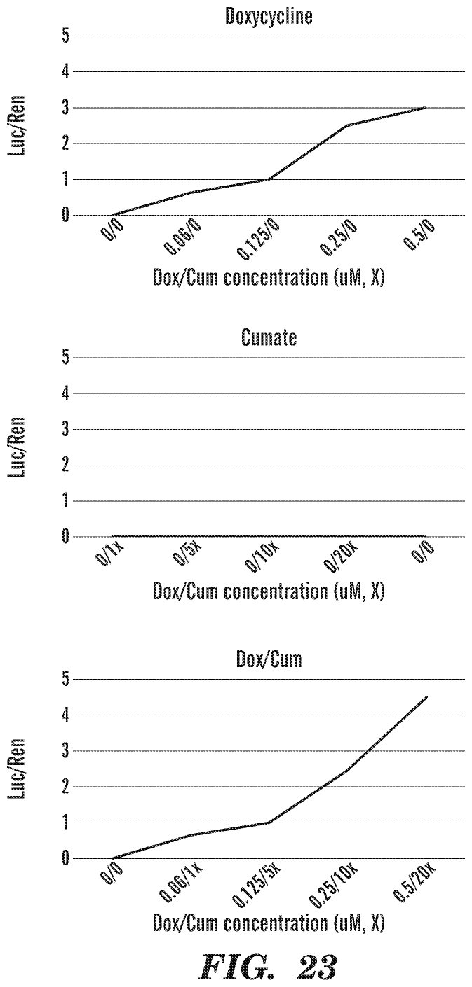

Finally, an experiment to validate the AND gate circuit was performed. HEK-293T-PB-Q-dCas9-VPR-Puro-pSB-TRE-gRNA1-Bla cells were seeded on 24-well plates (50,000 cells/well), transfected with P1-Luc and Renilla, and stimulated with doxycycline and cumate: 4 conditions (untreated, doxycycline, cumate, and cumate/doxycycline). Luciferase and Renilla were read 48 h post-stimulation (FIG. 23), confirming previous results (Table 5). All of the previous data are currently unpublished.

The logical next step is to replace the non-functioning reporter plasmid (P1-Luc). We have designed two strategies, both including a Switchgear Genomics goClone reporter, a ready-to-use plasmid that would include a BMP-2 promoter and Renilla gene¹⁴. This would allow us to clone one gRNA and target both an endogenous gene and the Renilla reporter simultaneously. A gRNA for the BMP-2 promoter has been designed, targeting a site <100 bp upstream of the transcription start site (TSS). With this vector (pSBtet-HH-gRNAb-HDV-Bla) cloned and the BMP-2 goClone reporter, we will transfect HEK-293T cells selected for PB and validate AND gate function. In parallel, a different inducible transposon has been designed using an optically-induced construct (blue light). Gibson assembly will be used to construct relevant sequences from HH-gRNAb-HDV, EL222 photoresponsive element, and pSBtet-Bla¹⁵.

TABLE 5
Observed truth table for CRISPR AND gate.

| Dox | Cum | Luc |
|---|---|---|
| 0 | 0 | 0 |
| 1 | 0 | 1 |
| 0 | 1 | 0 |
| 1 | 1 | 1 |

### Example 4

Notwithstanding the inherent regenerative capability of bone tissue, an estimated 100,000 fractures develop nonunion in the United States every year¹. Fracture nonunions are cases where the healing process is delayed, and are associated with prolonged disability and pain for the patient, while significantly increasing medical care costs. The gold standard biological stimulation technique for nonunion treatment is autogenous bone grafting, but associated drawbacks have driven the development of alternatives. Recombinant growth factors have been efficacious alternatives, but high cost and adverse effects linked to supra-physiological doses have prompted the investigation of gene therapy approaches that deliver constitutively expressed genes. CRISPR/Cas systems are used in some of these approaches, and are increasingly being considered for tissue engineering applications³. While promising, approaches where cells are constitutively over expressing transgenes raise several concerns, such as ectopic bone formation, inability to control release profiles, and inability to suppress transgene expression in heterotopic sites. Engineered cellular production of growth factors with regulation of spatio-temporal gene expression would allow for highly effective treatments, minimized complications, and biomimetic dynamic gene expression profiles⁴. Synthetic biology approaches that construct artificial gene regulatory networks (GRN) can provide tightly regulated growth factor production that responds to physico-chemical cues that are convenient to deliver and control⁵. The present study addresses the difficulties associated with recombinant protein and gene therapy by developing an inducible transposon and CRISPR-based logic AND gate for the controlled, localized delivery of endogenously produced BMP-2. The impact of an AND gate-controlled, external stimuli-responsive, and localized delivery of BMP-2 to bone tissue engineering is predicted to be significant, while also serving as a base for future development of more complex circuits to drive synthetic GRN-controlled recombinant protein therapy in tissue engineering and regenerative medicine.

TABLE 6
CRISPR AND gate input and output vectors.

| Input/Output | Vector |
|---|---|
| Cumate | pPBq-dCas9-VPR |
| Doxycycline | pSBtet-HH-gRNA-HDV-mCherry |
| Renilla/BMP-2 | goClone Reporter/Endogenous Gene |

### Example 5: CRISPR Activation of Endogenous Gene (BMP-2)

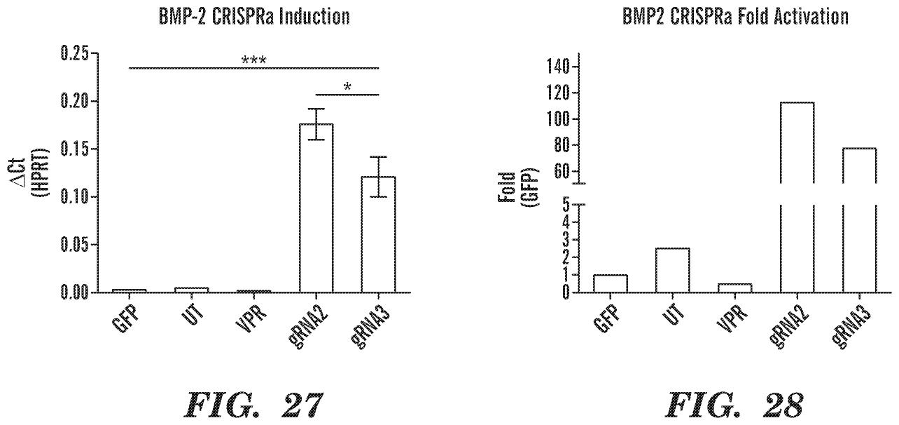

HEK-293T cells were transfected with constitutively-expressed dCas9-VPR (Addgene #63798) and gRNA-expressing plasmids (Addgene #41824, sequences targeting the 150 bp region of the transcription start site of the endogenous BMP-2 was cloned into the vector; gRNA2 seq: GGCGAGCCGCGCCGCGAAGG, gRNA3 seq: GCTCGCCGGGGATCCCCGAG, GFP seq:). After 24 hours, BMP-2 induction was measured by qPCR, following manufacturer protocols. Groups of HEK-293T cells tested were: Untransfected (UT), dCas9-VPR (VPR), GFP, gRNA2, and gRNA3. All groups were normalized to Hypoxanthine-guanine phosphoribosyltransferase (HPRT) expression (FIG. 27). Untransfected, dCas9-VPR, gRNA2, and gRNA3 groups were compared to GFP to elucidate increase in expression levels (FIG. 28).

The gRNA2 and gRNA3 groups showed 112 and 77-fold induction when compared to GFP group. These data show that the gRNAs designed can induce significant over expression of endogenous BMP-2.

### Example 6: An Embodiment of an Electrospun Template in Accordance with the Invention

Anterior cruciate ligament reconstruction surgery has high repair failure, mostly due to the current inability to regenerate a properly functioning enthesis¹⁹. The structural and mechanical gradients of entheses are critical for normal function and regenerating these gradients is a major challenge. The goal in this example is to develop a two-component system in which an electrospun template (component 1) will be used to deliver physicochemical cues to engineered adipose-derived stem cells (ASCs) (component 2) to generate a countergradient of growth factors that will guide the regeneration of the gradient structure of the enthesis. Air gap electrospinning uses grounded targets separated in space to collect fibers, resulting in aligned structures that mimic the native ligament. For the development of the delivery of gradient stimuli, the inventors chosed blue light, expecting diffusion through the template according to Beer-Lambert's Law.

Polycaprolactone (PCL) was dissolved overnight in 1,1,1,3,3,3 hexafluoro-2-propanol at 100 mg/mL concentration and electrospun (+25 KV, 10 cm, 3 ml/hr, collected on a rotating air gap mandrel). Fiber diameter was measured using scanning electron microscopy (SEM), and analyzed (n=300 measurements, FibraQuant™ 1.3 software). Uniaxial tensile testing (n=5) was performed with a TestResources™ frame (Model: 220Q). Dog-bone shaped samples were allowed to proceed to failure at a strain rate of 10 mm/min, and Force-Elongation curves recorded with XY software. To analyze the gradient in blue light intensity along the longitudinal axes of the templates, samples (n=5) were illuminated from one end with an OpalDrive laser source and Laser Wire® guide (470 nm wavelength) and images acquired with a Pariss Imaging Microscope. The intensity of each pixel was calculated with ImageJ software, and plotted along the longitudinal axis.

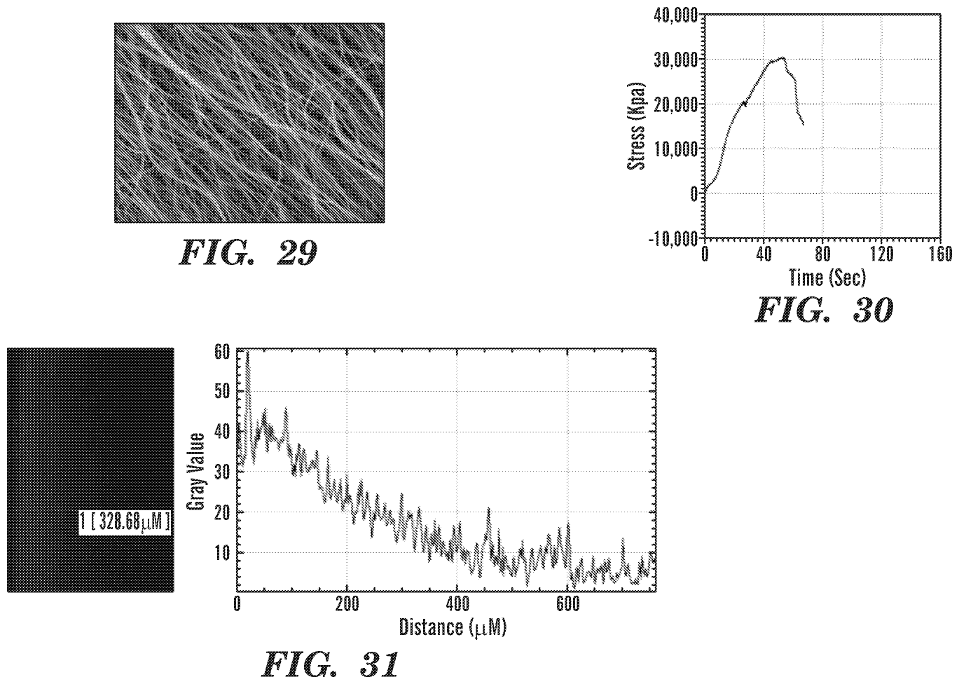

Templates had an average fiber diameter of 519 nm±X. Fibers were aligned and presented similar morphology to ligament ECM (FIG. 29). Uniaxial tensile testing showed that templates exhibit similar characteristics to natural pig ligament. Particularly interesting is the presence of an initial concave area, a linear range, and initial failure point in the force-elongation curves (FIG. 30). Blue light gradients were exhibited on the first 400 μm of the template, and correspond to the desired range of enthesis depth—200-500 μm (FIG. 31). The air gap electrospun templates have biomimetic characteristics (morphological and mechanical) and are amenable to the generation of a blue light gradient for optogenetic stimulation of growth factor production by engineered cells. This combination should be useful in the directed differentiation of cells to populate the enthesis. This study is the first to elucidate the possibility of presenting engineered cells with blue light stimulation in a gradient fashion for interfacial tissue engineering.

### Example 7: Flow Cytometry of pSBtet-mCherry-RGR-Bla Transfected HEK-293T Cells

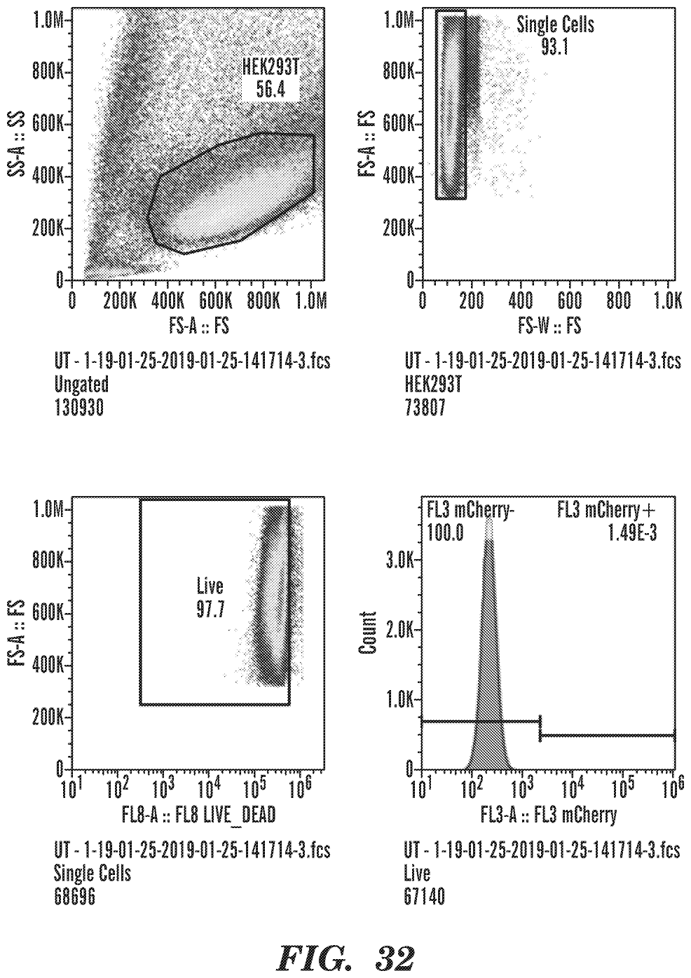

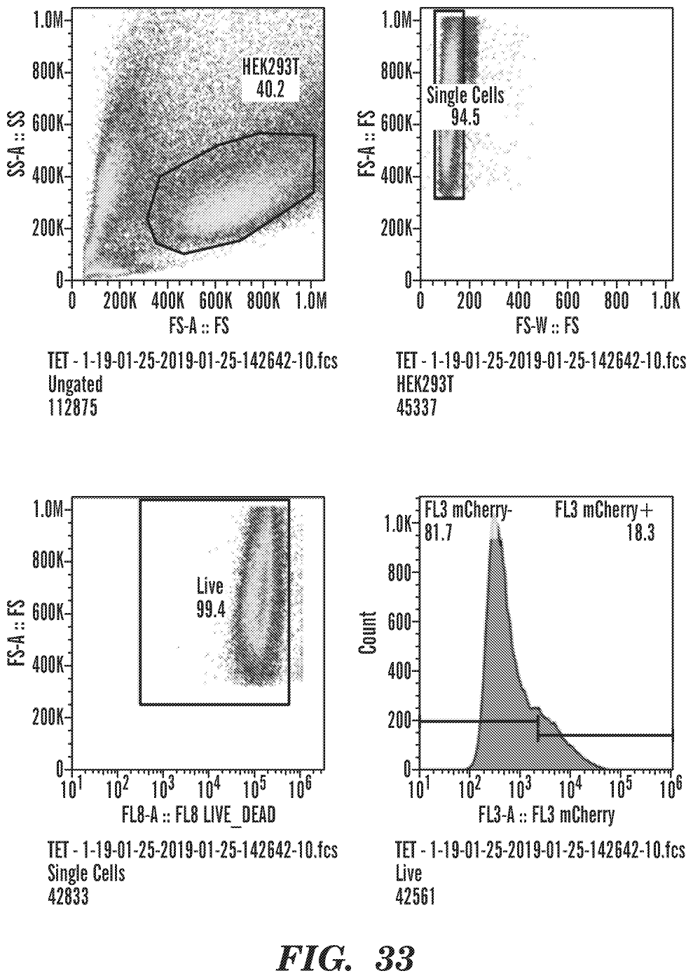

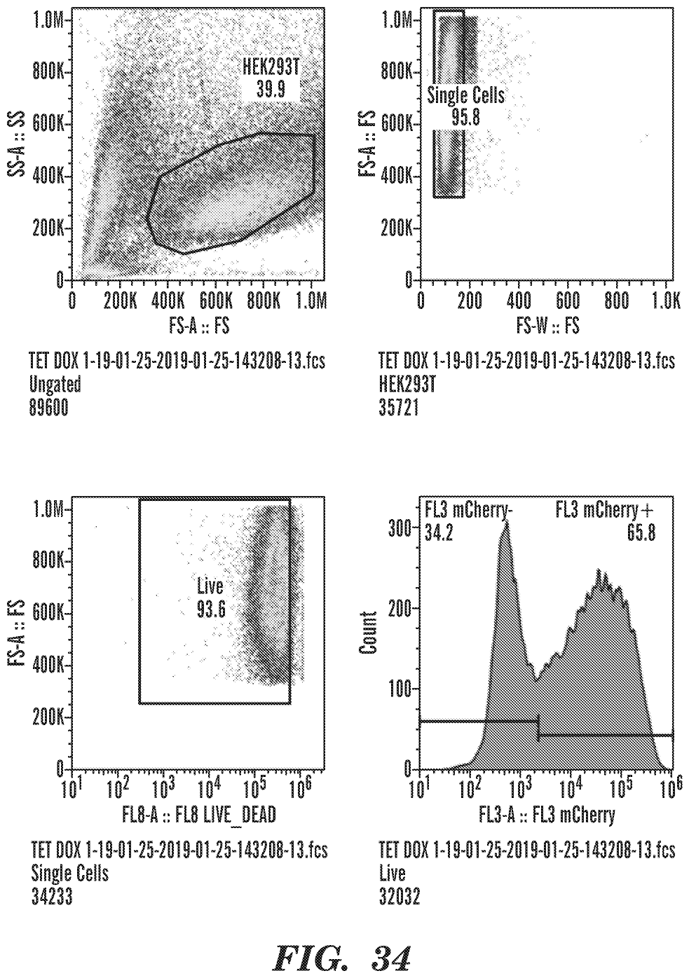

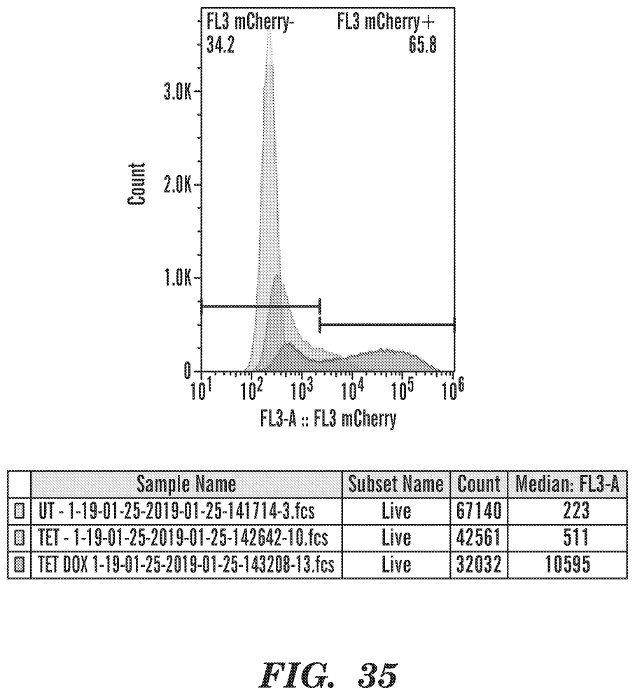

To assess inducibility of pSBtet-mCherry-RGR-Bla, flow cytometry was used to measure mCherry expression (median fluorescence intensity (MFI)). HEK-293T cells were seeded in a 6 well plate (2×10⁵ cells/well) and reverse transfected with pSBtet-mCherry-RGR-Bla plasmid (XtremeGene9 transfection reagent). Wild type cells were plated identically as negative controls, and transfected cells were labeled as TET (no stimulus) and TET DOX (doxycycline stimulus). Cells were incubated overnight at 37° C. and 24 hours post-transfection, TET DOX cells were stimulated with 1 μM Doxycycline. Flow cytometry with a Gallios instrument was performed 18 hours post-stimulation. Gating strategy and MFI measurements are illustrated in FIG. 32-35.

Doxycycline-stimulated cells exhibit a strong fluorescence, indicating successful activation and expression of the mCherry-RGR cassette. Unstimulated cells with the pSBtet-mCherry-RGR-Bla plasmid show slight leakiness, but it is expected that integration of the Sleeping Beauty transposon and titration of the doxycycline stimulus will reduce this leakiness.

### Materials and Methods for the Examples

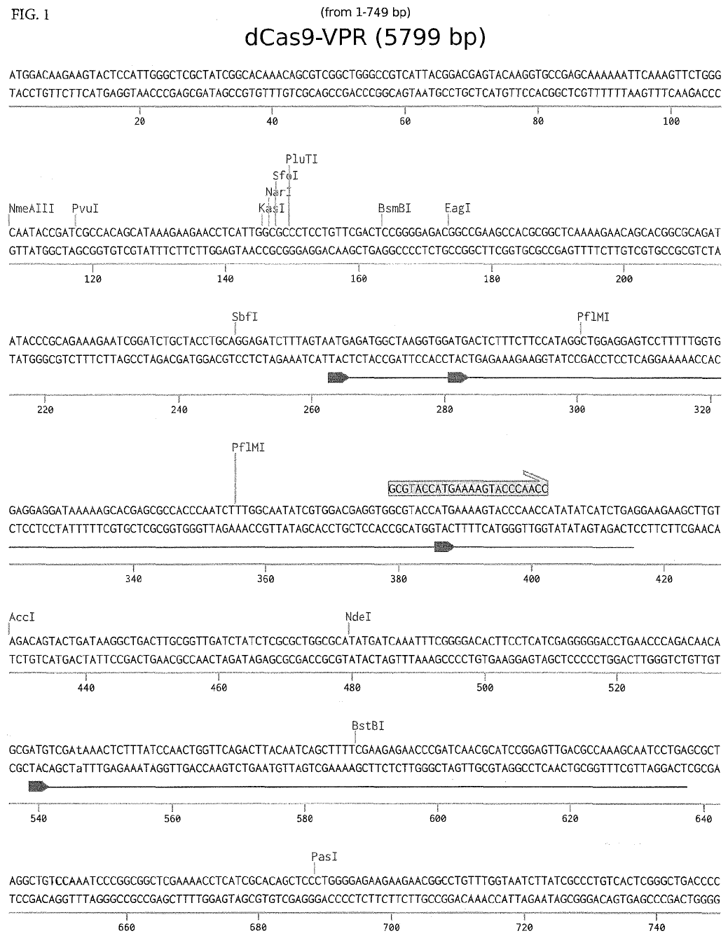

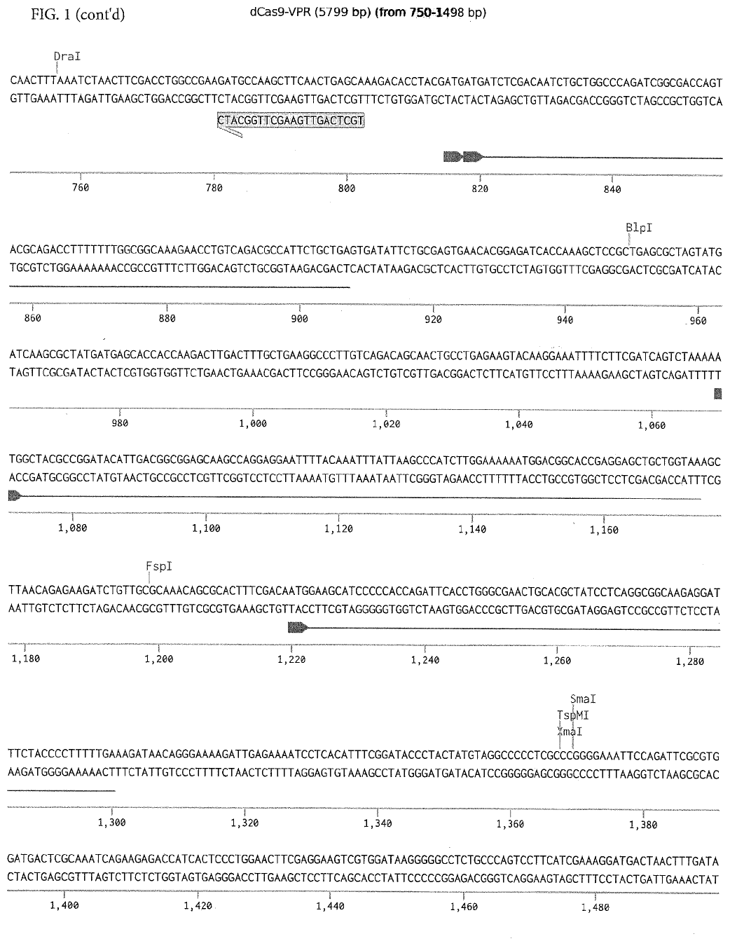

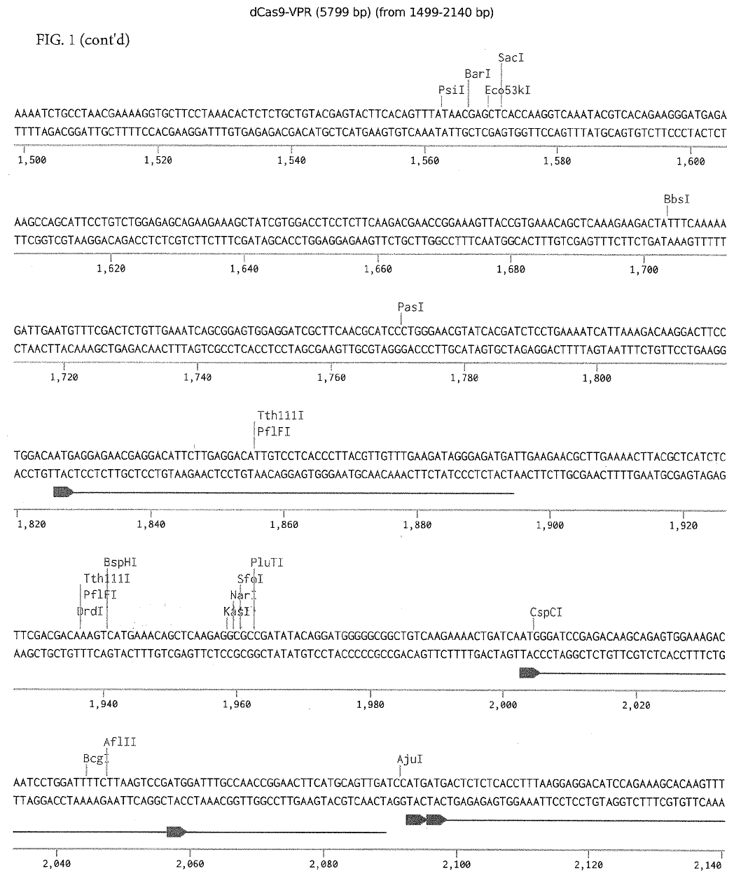

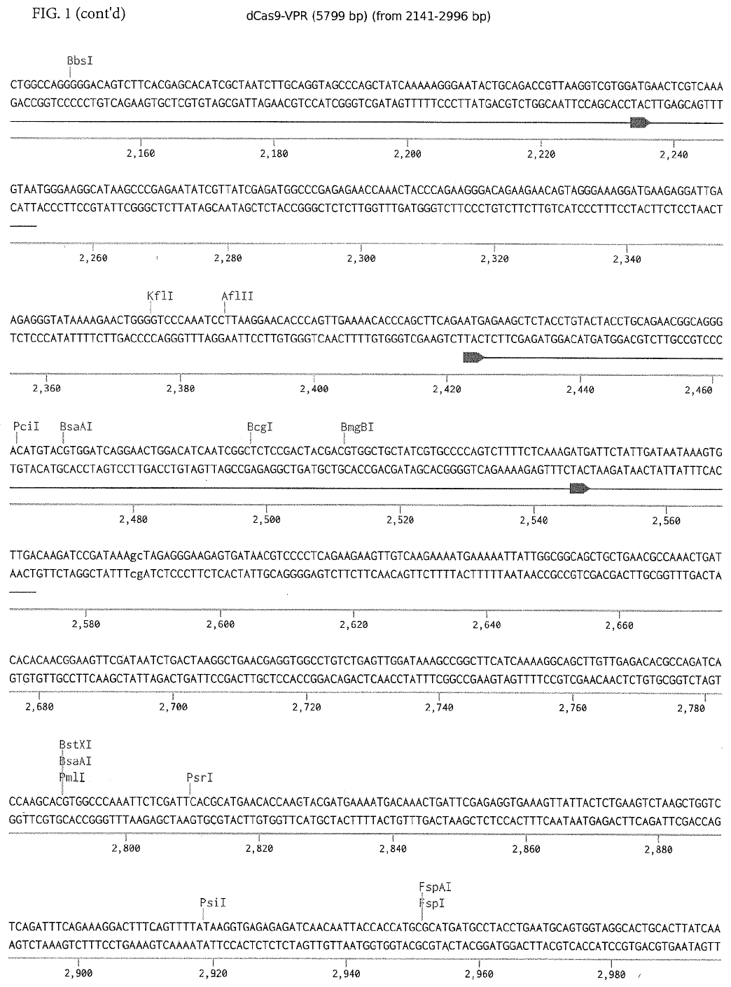

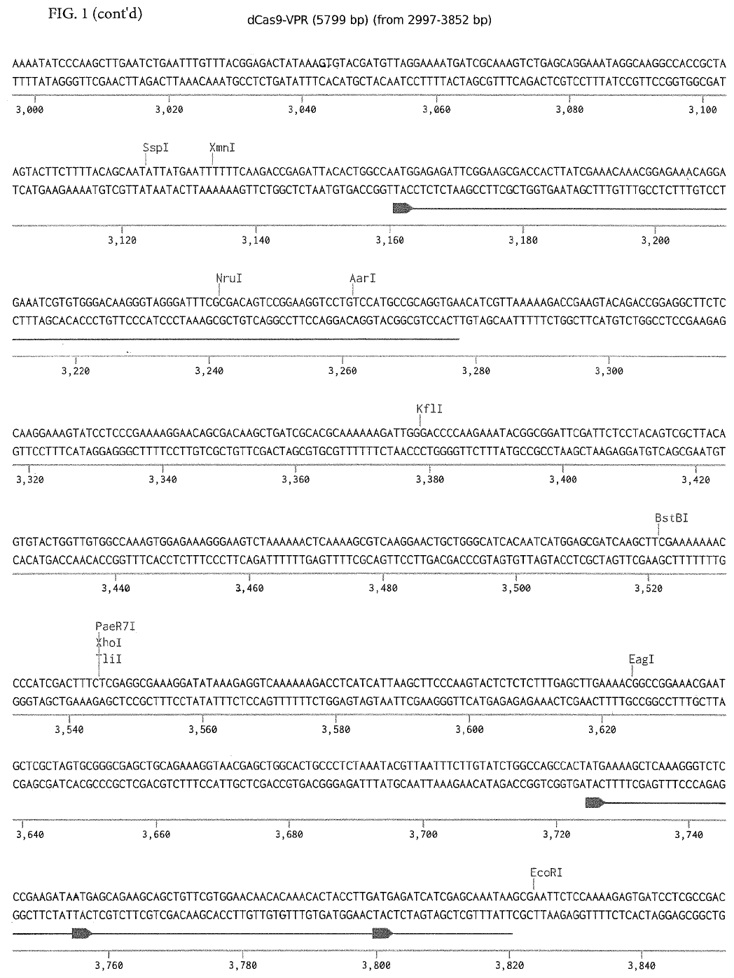

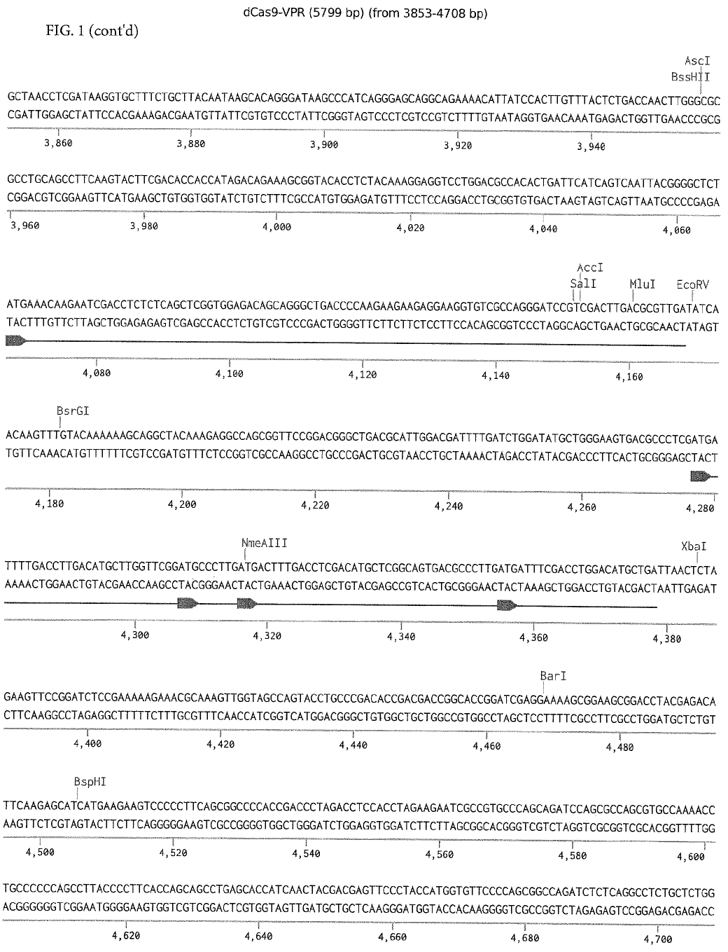

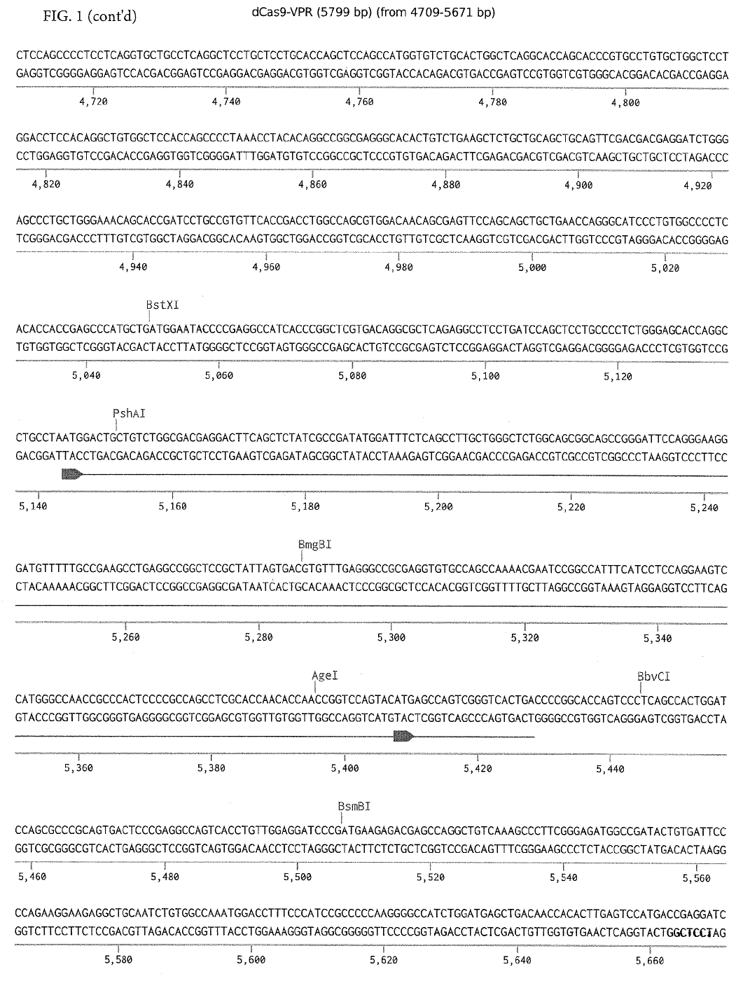

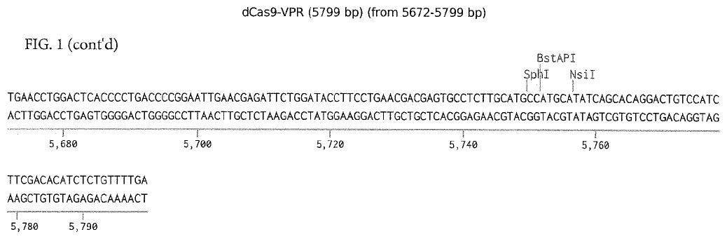

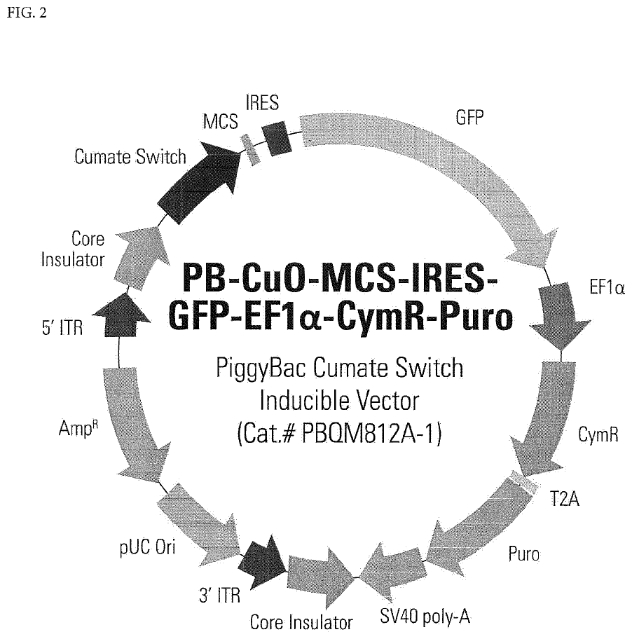

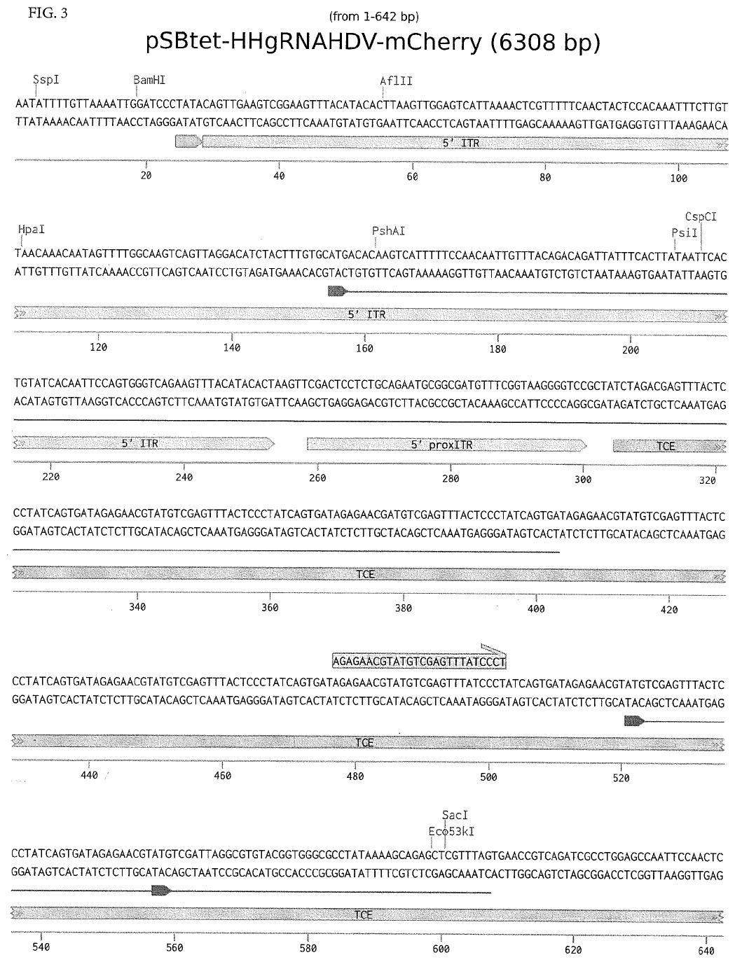

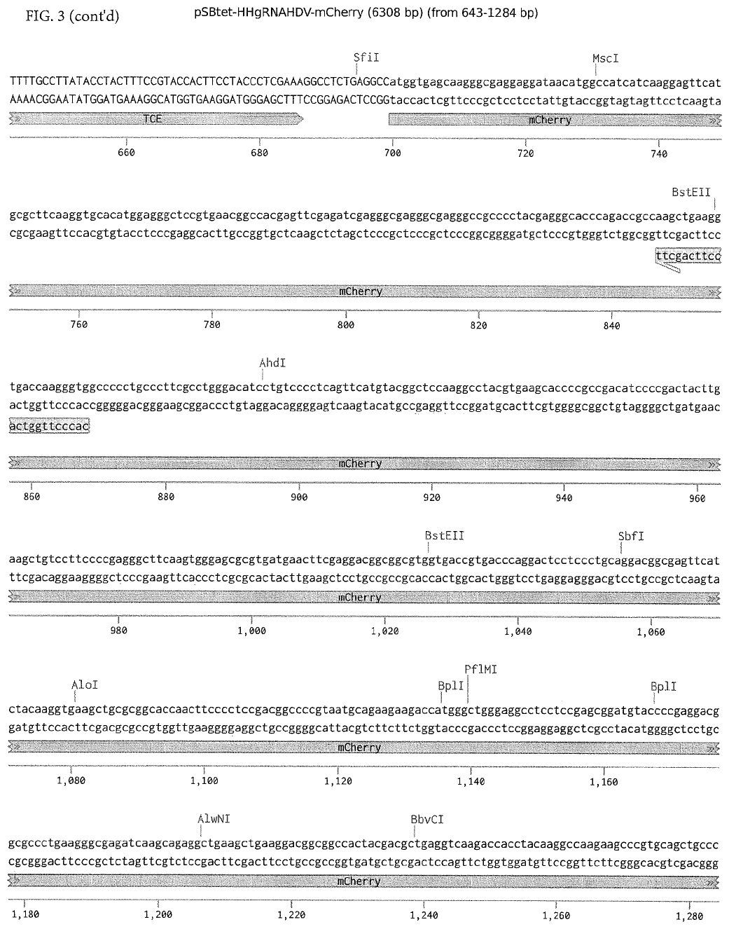

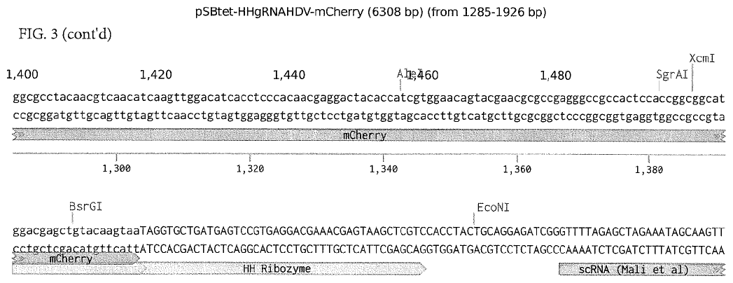

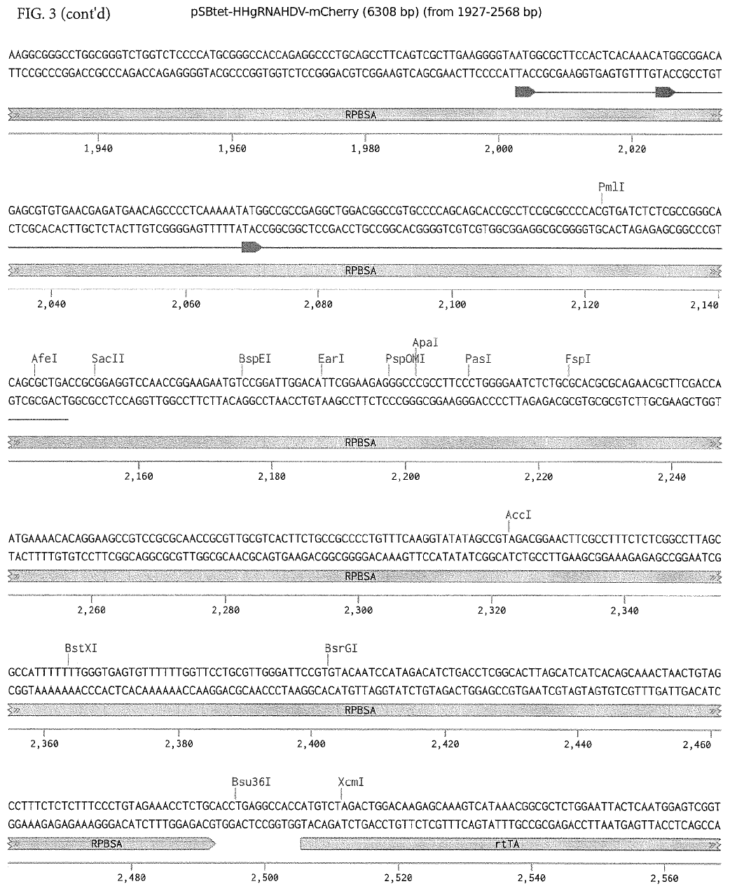

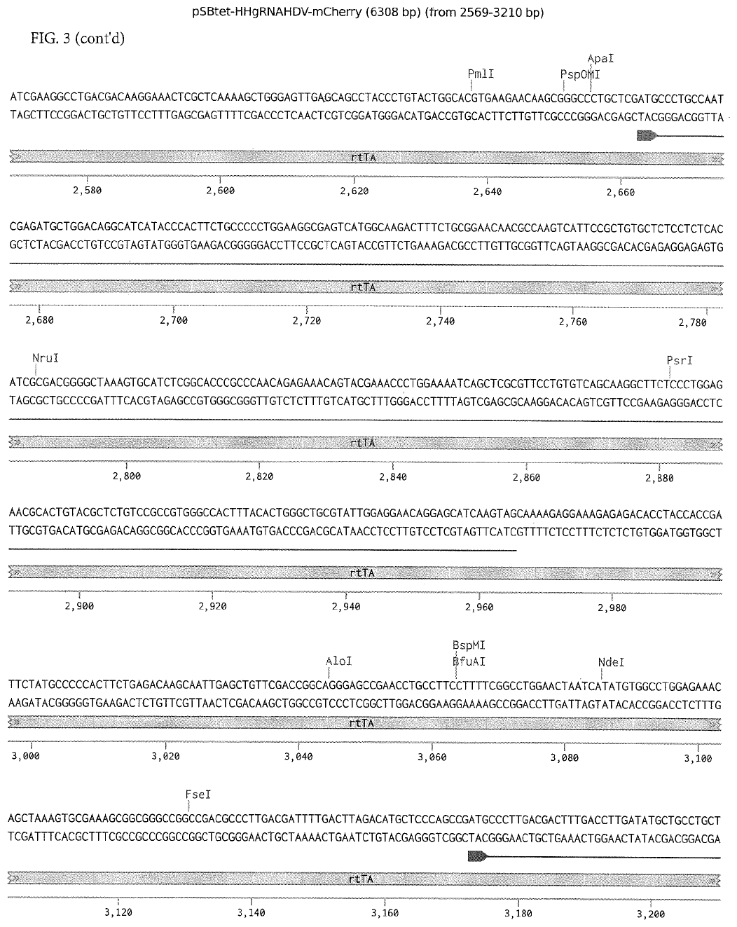

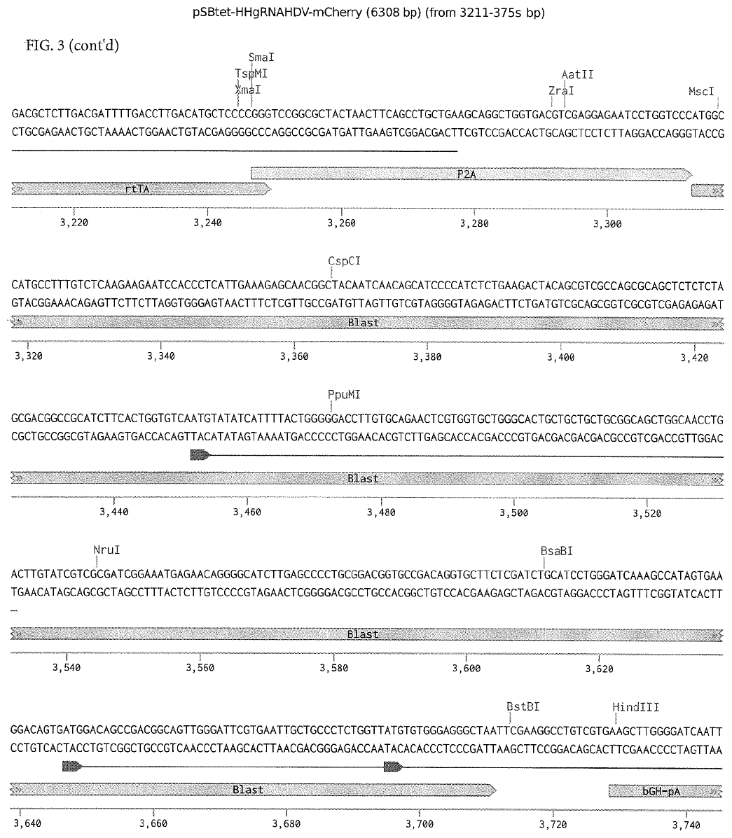

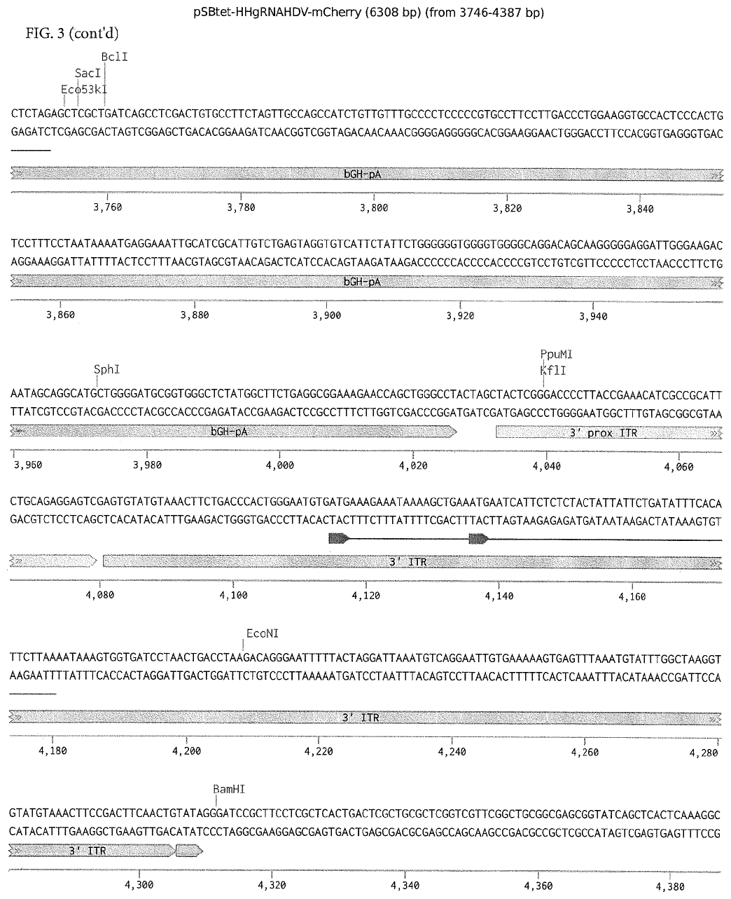

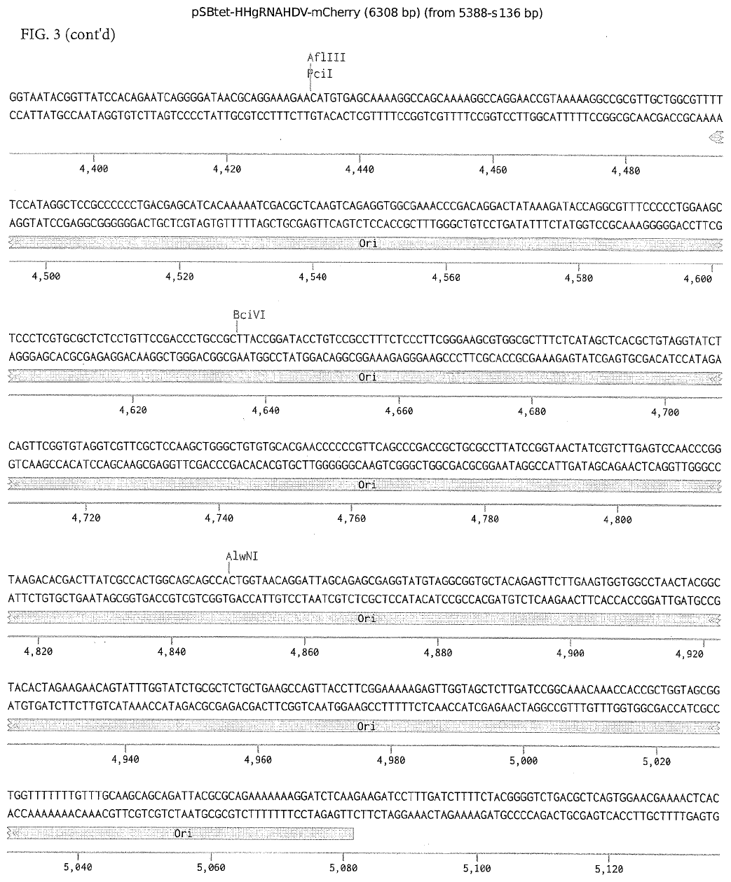

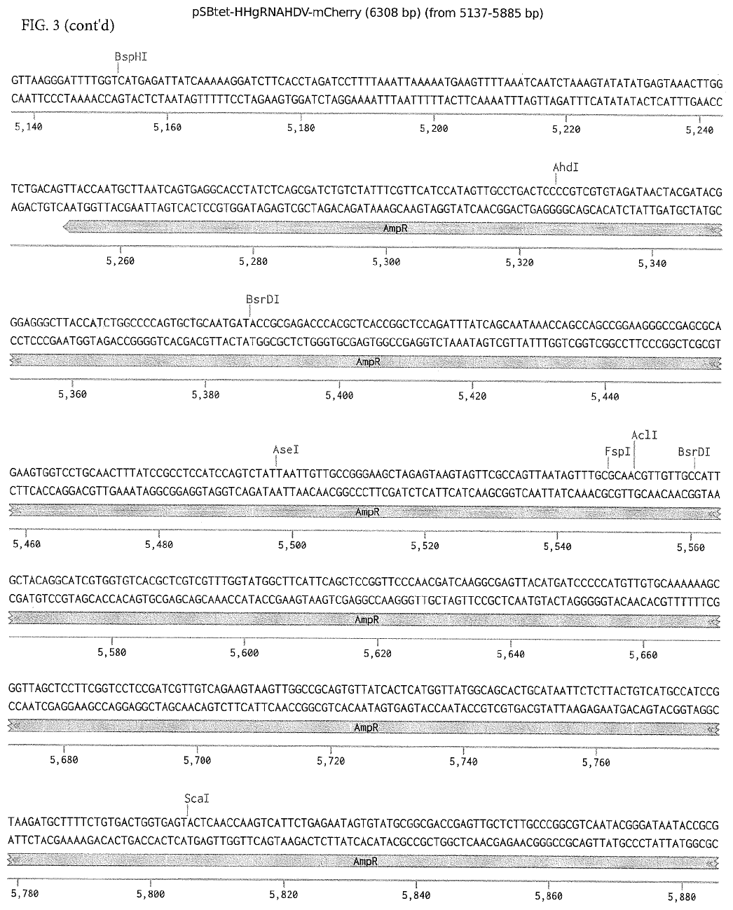

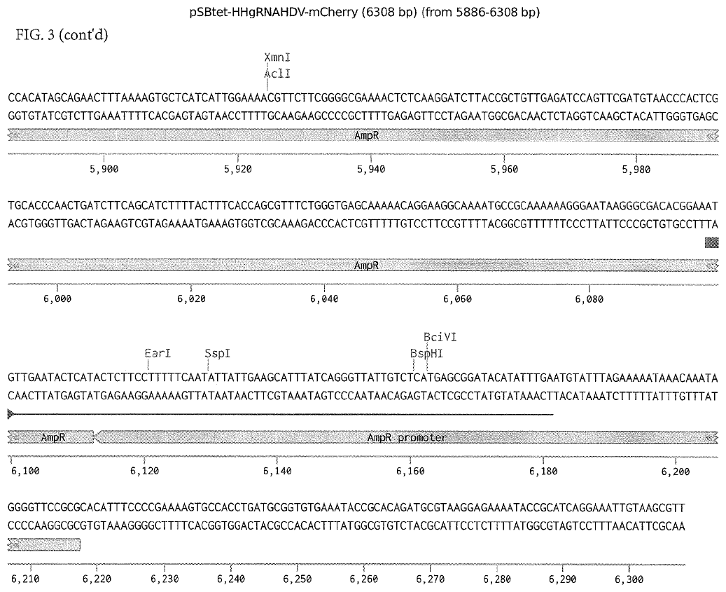

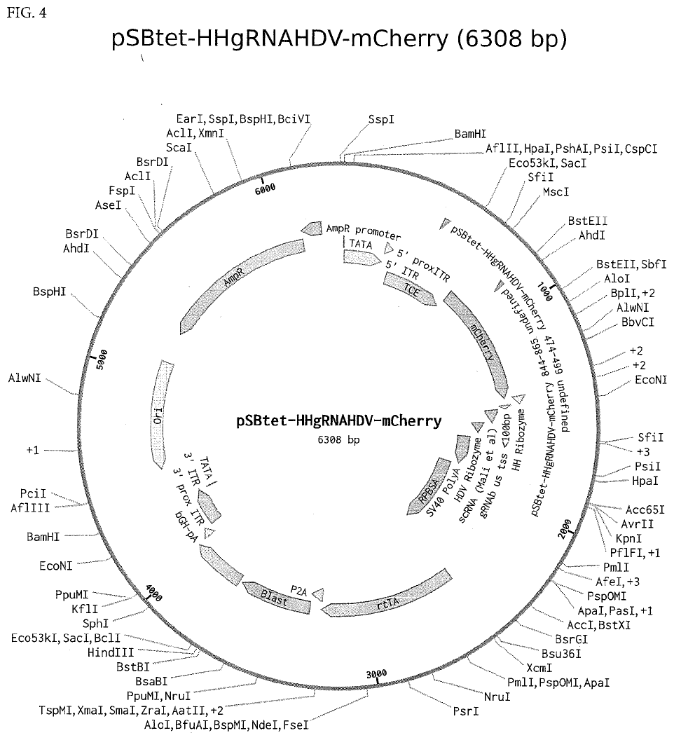

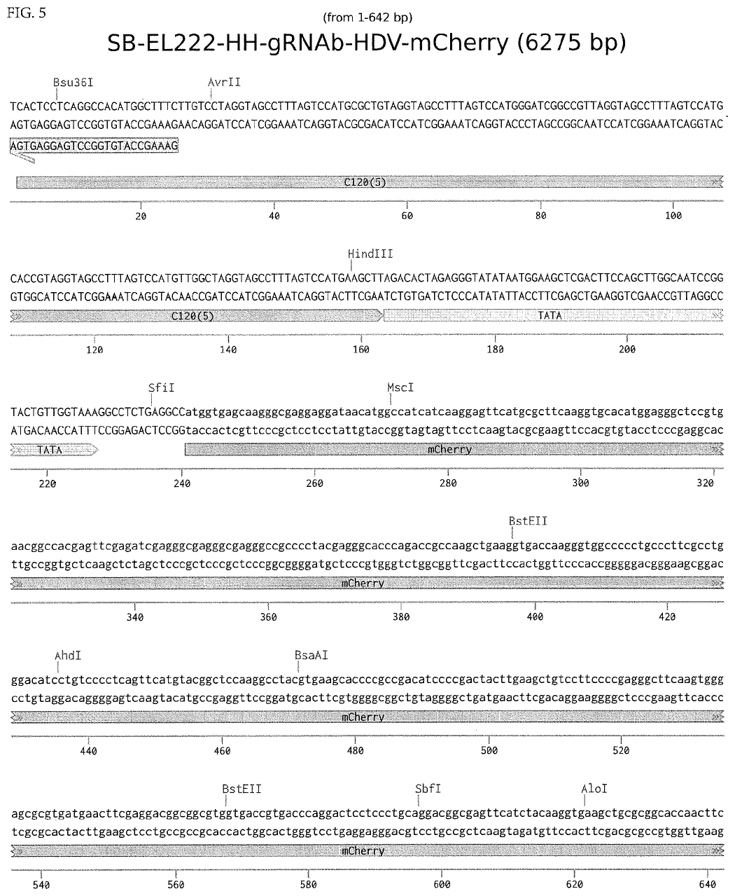

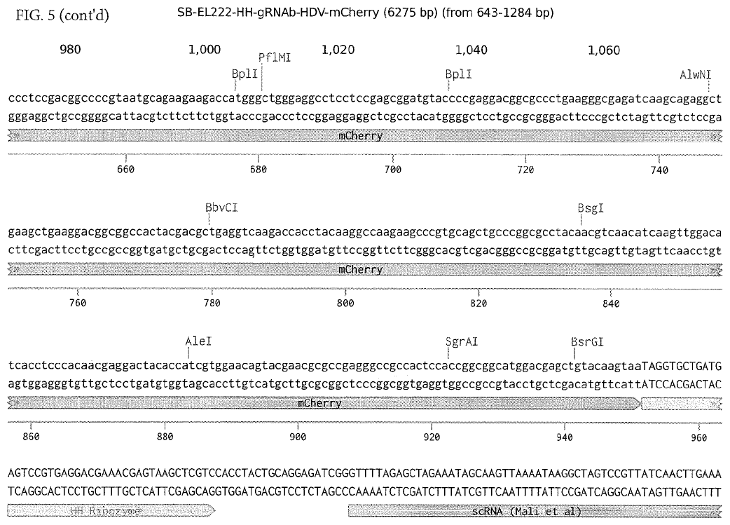

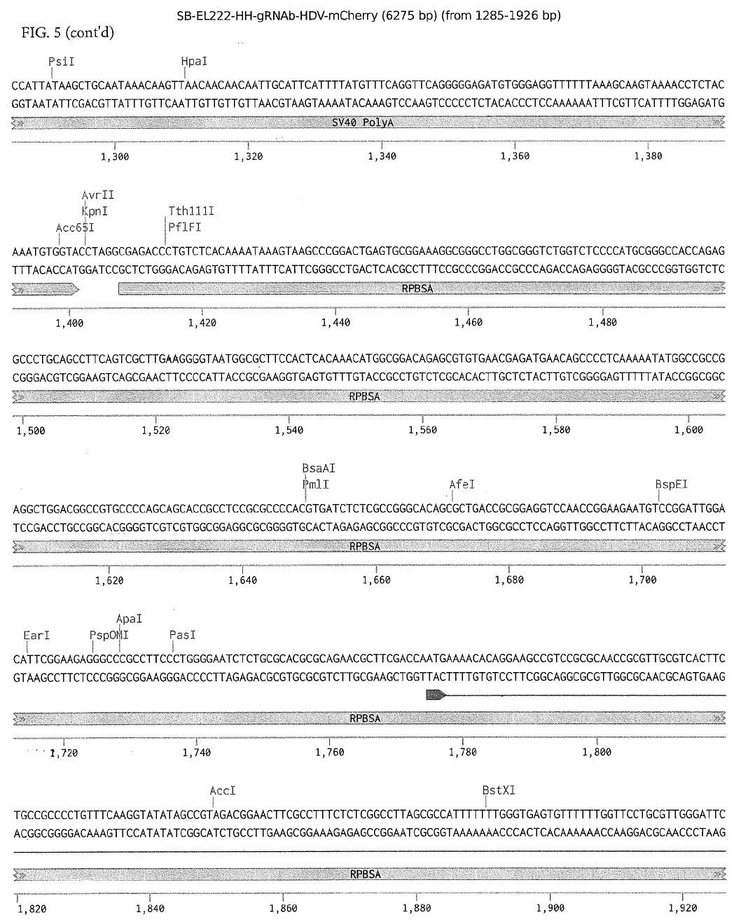

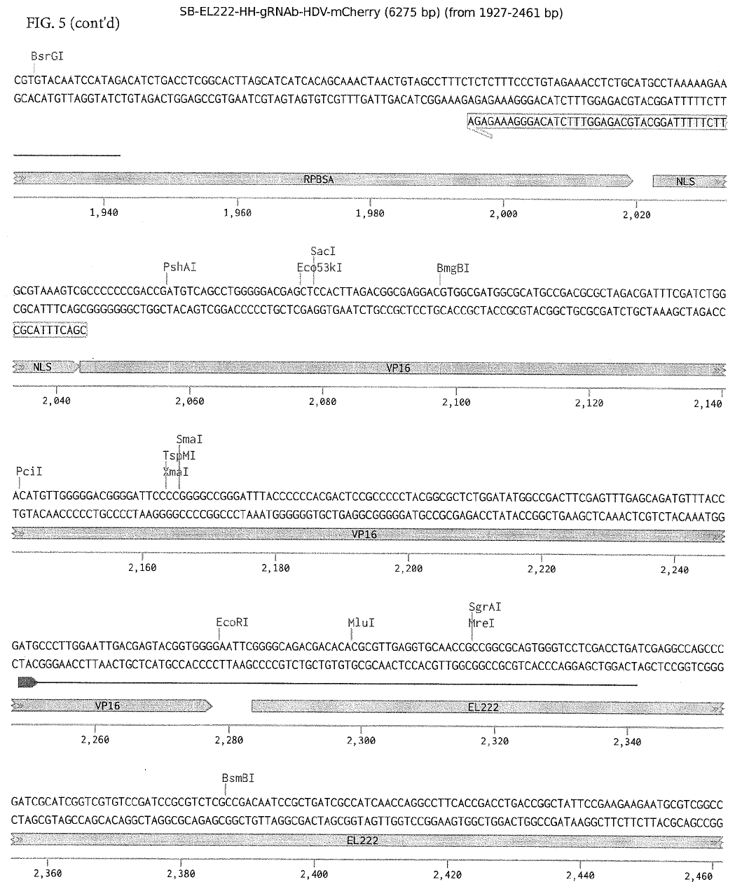

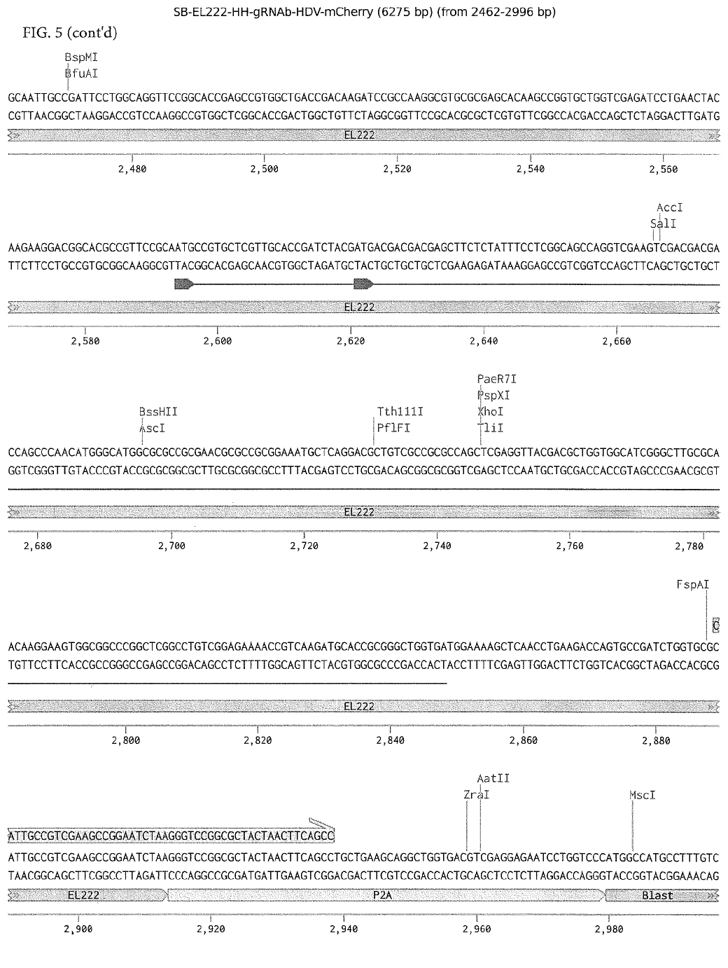

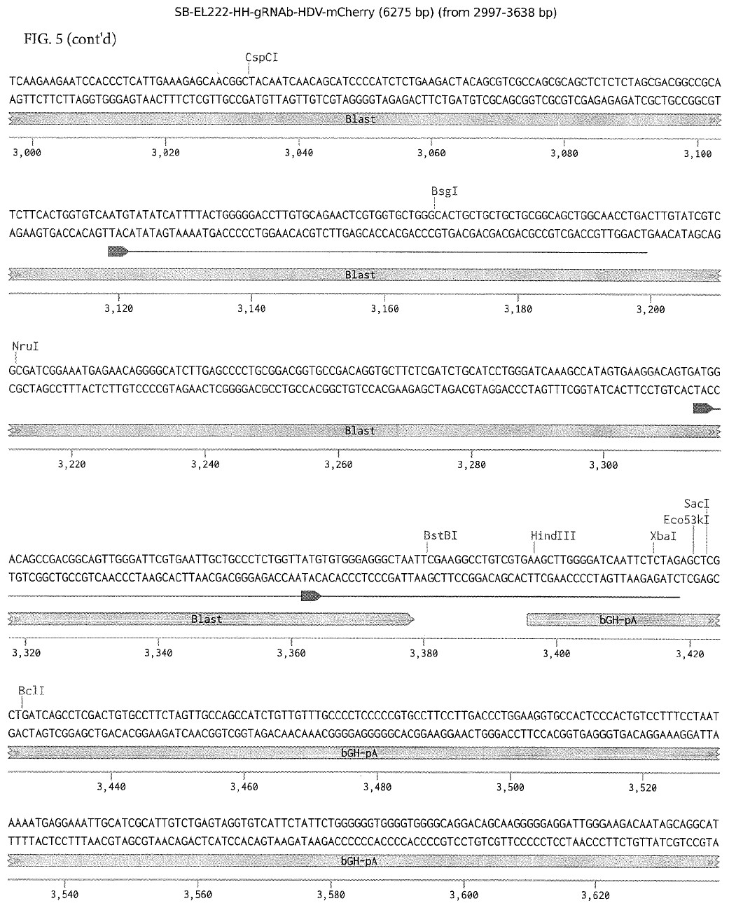

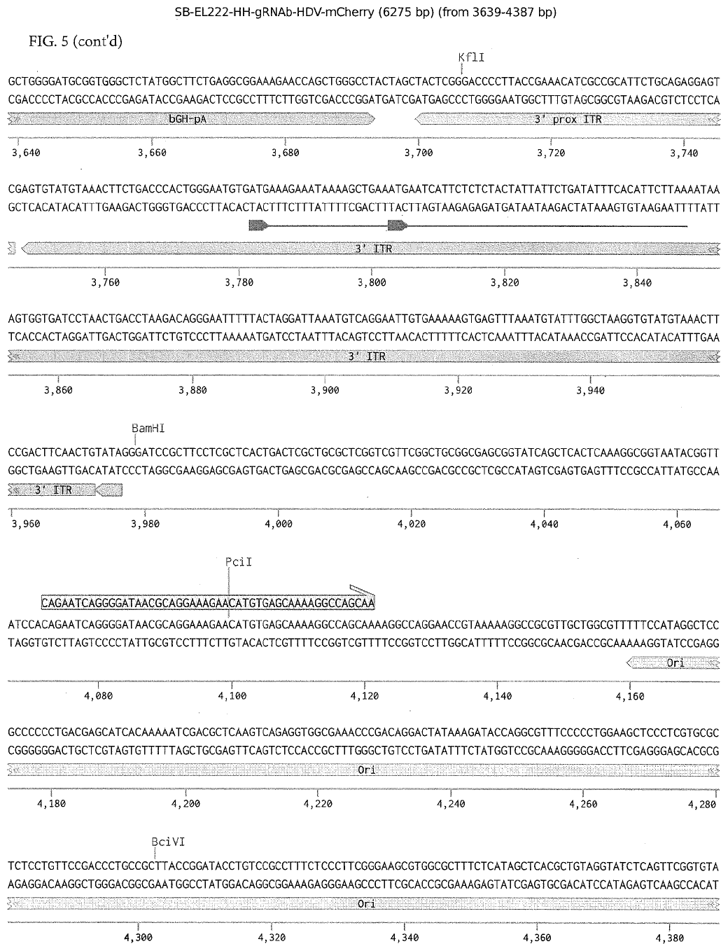

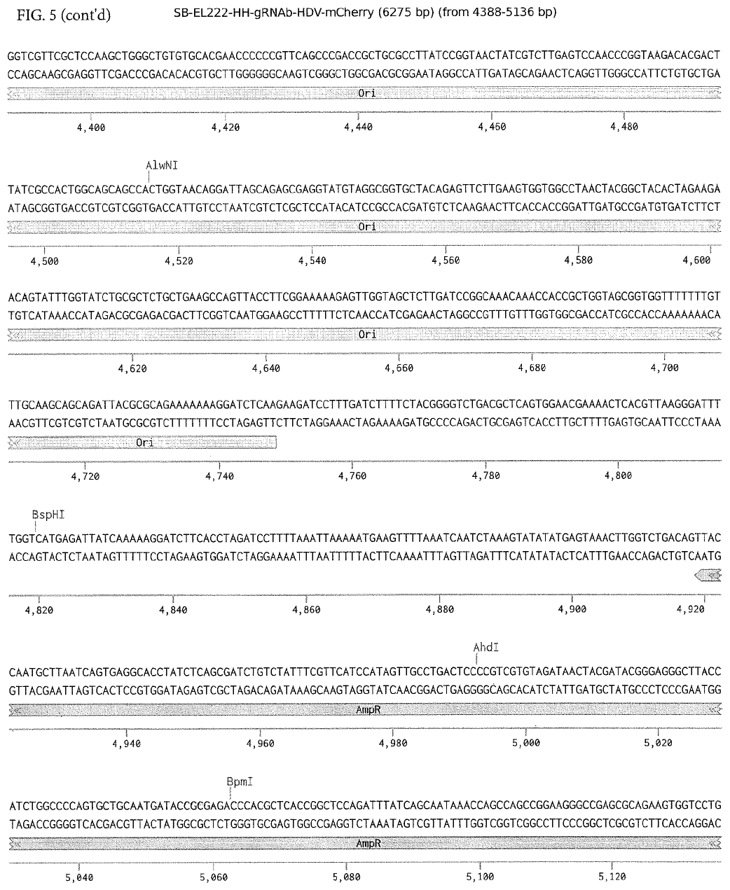

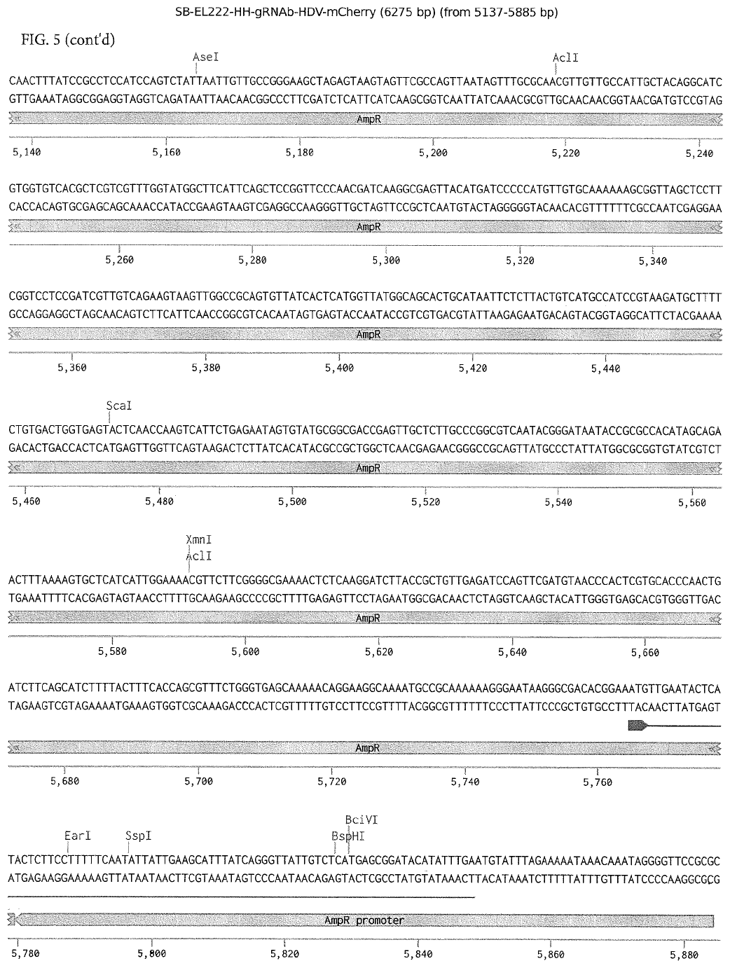

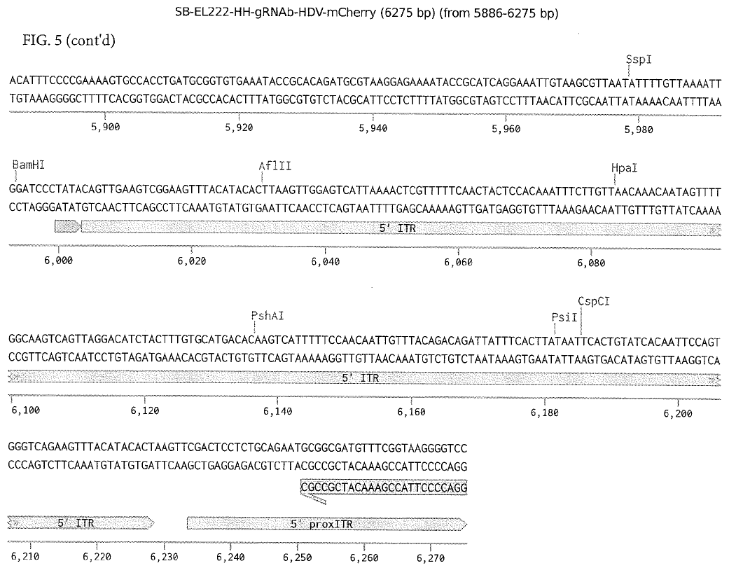

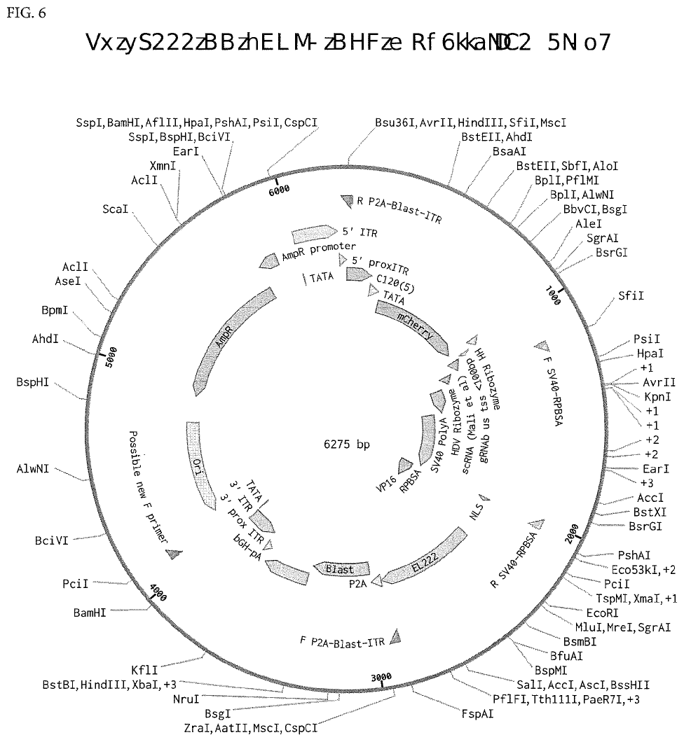

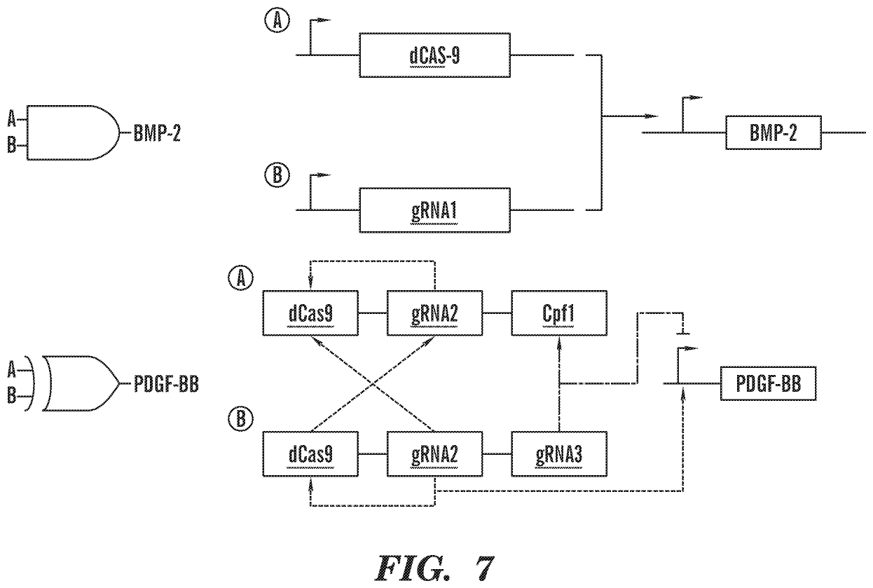

Sequences used to obtain the results described herein are known in the art or described, for example, at FIGS. 1-6.

gRNA Design

A gRNA targeting a site <100 base pairs upstream of the transcription start site (TSS) of human BMP-2 was designed in silico. The gRNA used is flanked by a Hammerhead ribozyme (HH) on the 5′ end and a Hepatitis Delta Virus ribozyme (HDV) on the 3′ end. These self-cleaving ribozymes allow the use of a Pol II promoter and a gRNA that does not start with a G nucleotide.

### Vectors

A logical AND gate was designed using inducible transposon dCas9 and gRNA constructs as inputs, and a promoter-luciferase reporter vector as output. The transposon system was chosen as it allows for large payloads and is usually less susceptible to epigenetic silencing when correctly integrated. The inputs are as follows: pSBtet-HH-gRNA-HDV-mCherry, a doxycycline-inducible Sleeping Beauty (SB) transposon with a blasticidin selection marker, mCherry, and a gRNA cloned into its multi-cloning site (MCS). The SB vector was a gift from Eric Kowarz (Goethe University, Germany, Addgene #60510) and the mCherry-HH-gRNA-HDV assembly was flanked with SfiI sites and synthesized by Integrated DNA Technologies (Coralville, Iowa) as a gBlock. Restriction enzyme cloning, with SfiI was used to insert this assembly into the SB vector. The second input is pPBq-dCas9-VPR, a cumate-inducible PiggyBac (PB) transposon from System Biosciences (Palo Alto, Calif., PBQM812A-1) with a puromycin selection marker and a dCas9-VPR cloned into its MCS. The dCas9-VPR was PCR amplified from a plasmid provided by George Church (Harvard University, Massachusetts, Addgene #63798) and cloned into the PB vector with TOPO cloning. The reporter vector is a Switchgear Genomics goClone reporter (SwitchGear Genomics, Menlo Park, Calif.).

TABLE 7
Expected truth table for CRISPR AND gate.

| Dox | Cum | Luc |
|---|---|---|
| 0 | 0 | 0 |
| 1 | 0 | 0 |
| 0 | 1 | 0 |

### Transfections

HEK-293T cells (ATCC, Manassas, Va.) were transfected with pSB-TRE-gRNA1-Bla, or pSBtet-HH-gRNA-HDV-mCherry, using X-tremeGENE 9 following manufacturer's protocol. Cells were selected 72 hours post-transfection with blasticidin at a concentration of 5 μg/ml for 5 days, generating a stable cell line labeled SB cells. SB cells were then transfected with pPBq-dCas9-VPR, and selected with a combination of blasticidin and puromycin at concentrations of 5 μg/ml and 3 μg/ml, respectively, for 3 days, generating a stable cell line labeled SB/PB cells.

### Input Function Tests

To test the functionality of pSBtet-HH-gRNA-HDV-mCherry and pPBq-dCas9-VPR co-expressed in a cell, inducibility of gRNA by doxycycline and dCas9-VPR was assayed, exploiting the presence of mCherry and EGFP, respectively. WT HEK-293T cells were used as a technical control. WT and SB/PB cells were seeded in a 24 well plate (0.5×10⁶ cells/well) and stimulated with doxycycline and cumate (0/0 and 1 μM/10× solutions). 24 hours later, cells were imaged using an Olympus microscope (Model BX43F) equipped with Olympus DP73 digital camera and fluorescent light source (mCherry excited at 587 nm, EGFP at 488 nm), 20× magnification, 250 ms exposure, with a bright field, GFP, and TRIT-C filter. Representative images were used for analysis.

### AND Gate Function Tests

The function of the AND gate circuit was assessed using a LightSwitch Luciferase Assay kit (SwitchGear Genomics, Menlo Park, Calif.). SB/PB cells were seeded on 96 well opaque white plates (5×10⁴ cells/well), transfected with BMP-2 goClone reporter vector, and stimulated with doxycycline and cumate to create 4 conditions (untreated, doxycycline (1 μM), cumate (10×) and doxycycline/cumate (1 μm/10×)), 3 samples per group. Luciferase activity was read 48 hours post-stimulation (SpectraMax i3, Molecular Devices, Sunnyvale, Calif.) measuring luminescence of all wavelengths, 2 seconds per well.

### Input Function Tests

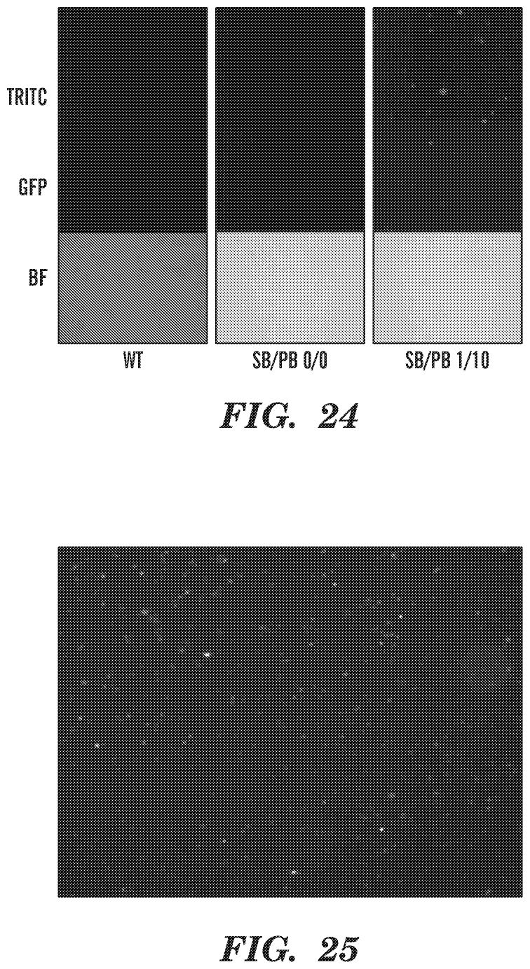

Both pSBtet-HH-gRNA-HDV-mCherry and pPBq-dCas9-VPR show inducibility by doxycycline and cumate, respectively (FIG. 24).

Fluorescence microscopy suggests little to no leakiness in both systems (FIG. 24). pSBtet-HH-gRNA-HDV-mCherry and pPBq-dCas9-VPR are co-expressed in cells exposed to both doxycycline and cumate after 24 hours (FIG. 25).

### AND Gate Function Tests

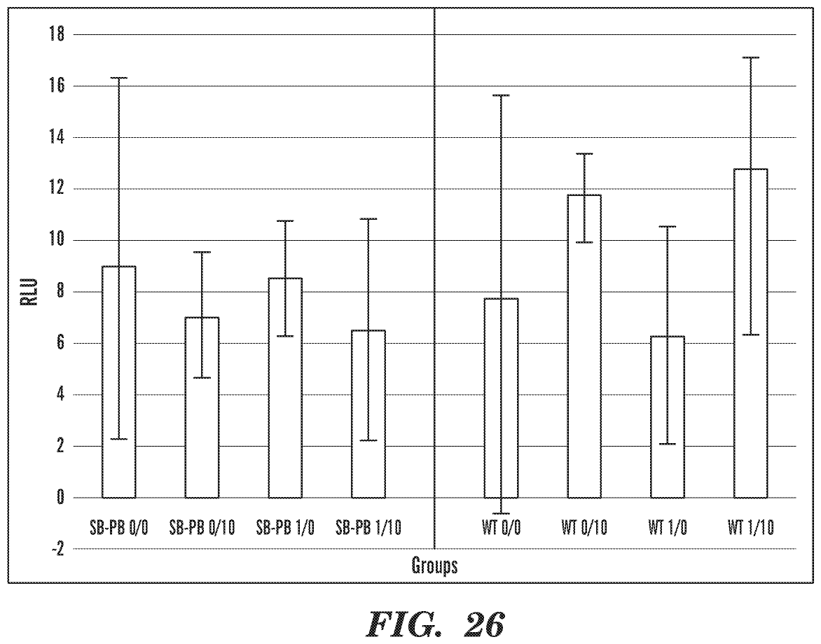

Luciferase assay results indicate that there are no responsive elements in the reporter plasmid that would react to the stimuli and mask AND gate behavior. (FIG. 26).

Luciferase activity was not observed in any of the groups (FIG. 26 and Table 8), suggesting a lack of transcription activation by the dCas9-VPR/gRNA complex. The experiment was repeated 6 times with comparable results.

TABLE 8
Observed truth table for CRISPR AND Gate

| Dox | Cum | Luc |
|---|---|---|
| 0 | 0 | 0 |
| 1 | 0 | 0 |
| 0 | 1 | 0 |
| 1 | 1 | 0 |

An inducible transposon and CRISPR-based AND gate targeting an endogenous gene (BMP-2) was designed and correct input function was confirmed.

Luciferase activity was not observed in the co-stimulated SB/PB cells, possibly a function of gRNA design.

The AND gate-controlled, localized delivery of BMP-2 is the first application of CRISPR-based logic gates in tissue engineering.

### REFERENCES

1. Hak, D. J. et al. Delayed union and nonunions: Epidemiology, clinical issues, and financial aspects. Injury 45, S3S7 (2014).
2. Antonova, E., Le, T. K., Burge, R. & Mershon, J. Tibia shaft fractures: costly burden of nonunions. BMC Musculoskelet. Disord. 14, 42 (2013).
3. Kanakaris, N. K. & Giannoudis, P. V. The health economics of the treatment of long-bone non-unions. Injury 38, S77S84 (2007).
4. Dimitriou, R. et al. Application of recombinant BMP-7 on persistent upper and lower limb non-unions. Injury 36, S51S59 (2005).
5. Gazdag, A. R., Lane, J. M., Glaser, D. & Forster, R. Alternatives to Autogenous Bone Graft: Efficacy and Indications. J. Am. Acad. Orthop. Surg. 3, 1-8 (1995).
6. Gómez-Barrena, E. et al. Bone fracture healing: Cell therapy in delayed unions and nonunions. Bone 70, 93-101 (2015).
7. Epstein, N. E. Complications due to the use of BMP/INFUSE in spine surgery: The evidence continues to mount. Surg. Neurol. Int. 4, S343S352 (2013).
8. Baltzer, A. W. A. et al. Genetic enhancement of fracture repair: healing of an experimental segmental defect by adenoviral transfer of the BMP-2 gene. Gene Ther. Houndmills 7, 734-9 (2000).
9. Lieberman, J. R. et al. Regional gene therapy with a BMP-2-producing murine stromal cell line induces heterotopic and orthotopic bone formation in rodents. J. Orthop. Res. 16, 330-339 (1998).
10. Tsuchida, H., Hashimoto, J., Crawford, E., Manske, P. & Lou, J. Engineered allogeneic mesenchymal stem cells repair femoral segmental defect in rats. J. Orthop. Res. 21, 44-53 (2003).
11. Lee, E. J., Tabor, J. J. & Mikos, A. G. Leveraging synthetic biology for tissue engineering applications. Inflamm. Regen. 34, 015-022 (2014).
12. Beal, J., Lu, T. & Weiss, R. Automatic Compilation from High-Level Biologically-Oriented Programming Language to Genetic Regulatory Networks. PLoS ONE 6, e22490 (2011).
13. Doudna, J. A. & Mali, P. CRISPR-Cas: a laboratory manual. (Cold Spring Harbor Laboratory Press, 2016).
14. Gilbert, L. A. CRISPR-mediated modular RNA-guided regulation of transcription in eukaryotes. Cell 154, 442-451 (2013).
15. Tanenbaum, M. E. A protein-tagging system for signal amplification in gene expression and fluorescence imaging. Cell 159, 635-646 (2014).
16. Zalatan, J. G. Engineering complex synthetic transcriptional programs with CRISPR RNA scaffolds. Cell 160, 339-350 (2015).
17. Kebriaei, P., Izsvak, Z., Narayanavari, S. A., Singh, H. & Ivies, Z. Gene Therapy with the Sleeping Beauty Transposon System. Trends Genet. 33, 852-870 (2017).
18. Gerber, H. P. et al. VEGF couples hypertrophic cartilage remodeling, ossification and angiogenesis during endochondral bone formation. Nat. Med. 5, 623-628 (1999).
19. Font Tellado, et al. Adv. Drug Deliv. Rev. 94, 126-140 (2015).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

Below are the sequences of the dCpf1-VPR and dCas9-VPR. The nucleolytically-inactive Cpf1 or Cas9 is fused with the chimeric activation domain VPR (composed of the activation domains of VP64, P65 and Rta).

dCpf1-VPR (LB)
atgagcaagctggagaagtttacaaactgctactccctgtctaagaccct
gaggttcaaggccatccctgtgggcaagacccaggagaacatcgacaata
agcggctgctggtggaggacgagaagagagccgaggattataagggcgtg
aagaagctgctggatcgctactatctgtcttttatcaacgacgtgctgca
cagcatcaagctgaagaatctgaacaattacatcagcctgttccggaaga
aaaccagaaccgagaaggagaataaggagctggagaacctggagatcaat
ctgcggaaggagatcgccaaggccttcaagggcaacgagggctacaagtc
cctgtttaagaaggatatcatcgagacaatcctgccagagttcctggacg
ataaggacgagatcgccctggtgaacagcttcaatggctttaccacagcc
ttcaccggcttctttgataacagagagaatatgttttccgaggaggccaa
gagcacatccatcgccttcaggtgtatcaacgagaatctgacccgctaca
tctctaatatggacatcttcgagaaggtggacgccatctttgataagcac
gaggtgcaggagatcaaggagaagatcctgaacagcgactatgatgtgga
ggatttctttgagggcgagttctttaactttgtgctgacacaggagggca
tcgacgtgtataacgccatcatcggcggcttcgtgaccgagagcggcgag
aagatcaagggcctgaacgagtacatcaacctgtataatcagaaaaccaa
gcagaagctgcctaagtttaagccactgtataagcaggtgctgagcgatc
gggagtctctgagcttctacggcgagggctatacatccgatgaggaggtg
ctggaggtgtttagaaacaccctgaacaagaacagcgagatcttcagctc
catcaagaagctggagaagctgttcaagaattttgacgagtactctagcg
ccggcatctttgtgaagaacggccccgccatcagcacaatctccaaggat
atcttcggcgagtggaacgtgatccgggacaagtggaatgccgagtatga
cgatatccacctgaagaagaaggccgtggtgaccgagaagtacgaggacg
atcggagaaagtccttcaagaagatcggctccttttctctggagcagctg
caggagtacgccgacgccgatctgtctgtggtggagaagctgaaggagat
catcatccagaaggtggatgagatctacaaggtgtatggctcctctgaga
agctgttcgacgccgattttgtgctggagaagagcctgaagaagaacgac
gccgtggtggccatcatgaaggacctgctggattctgtgaagagcttcga
gaattacatcaaggccttctttggcgagggcaaggagacaaacagggacg
agtccttctatggcgattttgtgctggcctacgacatcctgctgaaggtg
gaccacatctacgatgccatccgcaattatgtgacccagaagccctactc
taaggataagttcaagctgtattttcagaaccctcagttcatgggcggct
gggacaaggataaggagacagactatcgggccaccatcctgagatacggc
tccaagtactatctggccatcatggataagaagtacgccaagtgcctgca
gaagatcgacaaggacgatgtgaacggcaattacgagaagatcaactata
agctgctgcccggccctaataagatgctgccaaaggtgttcttttctaag
aagtggatggcctactataaccccagcgaggacatccagaagatctacaa
gaatggcacattcaagaagggcgatatgtttaacctgaatgactgtcaca
agctgatcgacttctttaaggatagcatctcccggtatccaaagtggtcc
aatgcctacgatttcaacttttctgagacagagaagtataaggacatcgc
cggcttttacagagaggtggaggagcagggctataaggtgagcttcgagt
ctgccagcaagaaggaggtggataagctggtggaggagggcaagctgtat
atgttccagatctataacaaggacttttccgataagtctcacggcacacc
caatctgcacaccatgtacttcaagctgctgtttgacgagaacaatcacg
gacagatcaggctgagcggaggagcagagctgttcatgaggcgcgcctcc
ctgaagaaggaggagctggtggtgcacccagccaactcccctatcgccaa
caagaatccagataatcccaagaaaaccacaaccctgtcctacgacgtgt
ataaggataagaggttttctgaggaccagtacgagctgcacatcccaatc
gccatcaataagtgccccaagaacatcttcaagatcaatacagaggtgcg
cgtgctgctgaagcacgacgataacccctatgtgatcggcatcgccaggg
gcgagcgcaatctgctgtatatcgtggtggtggacggcaagggcaacatc
gtggagcagtattccctgaacgagatcatcaacaacttcaacggcatcag
gatcaagacagattaccactctctgctggacaagaaggagaaggagaggt
tcgaggcccgccagaactggacctccatcgagaatatcaaggagctgaag
gccggctatatctctcaggtggtgcacaagatctgcgagctggtggagaa
gtacgatgccgtgatcgccctggaggacctgaactctggctttaagaata
gccgcgtgaaggtggagaagcaggtgtatcagaagttcgagaagatgctg
atcgataagctgaactacatggtggacaagaagtctaatccttgtgcaac
aggcggcgccctgaagggctatcagatcaccaataagttcgagagcttta
agtccatgtctacccagaacggcttcatcttttacatccctgcctggctg
acatccaagatcgatccatctaccggctttgtgaacctgctgaaaaccaa
gtataccagcatcgccgattccaagaagttcatcagctcctttgacagga
tcatgtacgtgcccgaggaggatctgttcgagtttgccctggactataag
aacttctctcgcacagacgccgattacatcaagaagtggaagctgtactc
ctacggcaaccggatcagaatcttccggaatcctaagaagaacaacgtgt
tcgactgggaggaggtgtgcctgaccagcgcctataaggagctgttcaac
aagtacggcatcaattatcagcagggcgatatcagagccctgctgtgcga
gcagtccgacaaggccttctactctagctttatggccctgatgagcctga
tgctgcagatgcggaacagcatcacaggccgcaccgacgtggattttctg
atcagccctgtgaagaactccgacggcatcttctacgatagccggaacta
tgaggcccaggagaatgccatcctgccaaagaacgccgacgccaatggcg
cctataacatcgccagaaaggtgctgtgggccatcggccagttcaagaag
gccgaggacgagaagctggataaggtgaagatcgccatctctaacaagga
gtggctggagtacgcccagaccagcgtgaagcacaaaaggccggcggcca
cgaaaaaggccggccaggcaaaaaagaaaaagggatcctacccatacgat
gttccagattacgcttatccctacgacgtgcctgattatgcatacccata
tgatgtccccgactatgccggaagcgaggccagcggttccggacgggctg
acgcattggacgattttgatctggatatgctgggaagtgacgccctcgat
gattttgaccttgacatgcttggttcggatgcccttgatgactttgacct
cgacatgctcggcagtgacgcccttgatgatttcgacctggacatgctga
ttaactctagaagttccggatctccgaaaaagaaacgcaaagttggtagc
cagtacctgcccgacaccgacgaccggcaccggatcgaggaaaagcggaa
gcggacctacgagacattcaagagcatcatgaagaagtcccccttcagcg
gccccaccgaccctagacctccacctagaagaatcgccgtgcccagcaga
tccagcgccagcgtgccaaaacctgccccccagccttaccccttcaccag
cagcctgagcaccatcaactacgacgagttccctaccatggtgttcccca
gcggccagatctctcaggcctctgctctggctccagcccctcctcaggtg
ctgcctcaggctcctgctcctgcaccagctccagccatggtgtctgcact
ggctcaggcaccagcacccgtgcctgtgctggctcctggacctccacagg
ctgtggctccaccagcccctaaacctacacaggccggcgagggcacactg
tctgaagctctgctgcagctgcagttcgacgacgaggatctgggagccct
gctgggaaacagcaccgatcctgccgtgttcaccgacctggccagcgtgg
acaacagcgagttccagcagctgctgaaccagggcatccctgtggcccct
cacaccaccgagcccatgctgatggaataccccgaggccatcacccggct
cgtgacaggcgctcagaggcctcctgatccagctcctgcccctctgggag
caccaggcctgcctaatggactgctgtctggcgacgaggacttcagctct
atcgccgatatggatttctcagccttgctgggctctggcagcggcagccg
ggattccagggaagggatgtttttgccgaagcctgaggccggctccgcta
ttagtgacgtgtttgagggccgcgaggtgtgccagccaaaacgaatccgg
ccatttcatcctccaggaagtccatgggccaaccgcccactccccgccag
cctcgcaccaacaccaaccggtccagtacatgagccagtcgggtcactga
ccccggcaccagtccctcagccactggatccagcgcccgcagtgactccc
gaggccagtcacctgttggaggatcccgatgaagagacgagccaggctgt
caaagcccttcgggagatggccgatactgtgattccccagaaggaagagg
ctgcaatctgtggccaaatggacctttcccatccgcccccaaggggccat
ctggatgagctgacaaccacacttgagtccatgaccgaggatctgaacct
ggactcacccctgaccccggaattgaacgagattctggataccttcctga
acgacgagtgcctcttgcatgccatgcatatcagcacaggactgtccatc
ttcgacacatctctgtttta
dCas 9-VPR (SP)
tggacaagaagtactccattgggctcgctatcggcacaaacagcgtcggc
tgggccgtcattacggacgagtacaaggtgccgagcaaaaaattcaaagt
tctgggcaataccgatcgccacagcataaagaagaacctcattggcgccc
tcctgttcgactccggggagacggccgaagccacgcggctcaaaagaaca
gcacggcgcagatatacccgcagaaagaatcggatctgctacctgcagga
gatctttagtaatgagatggctaaggtggatgactctttcttccataggc
tggaggagtcctttttggtggaggaggataaaaagcacgagcgccaccca
atctttggcaatatcgtggacgaggtggcgtaccatgaaaagtacccaac
catatatcatctgaggaagaagcttgtagacagtactgataaggctgact
tgcggttgatctatctcgcgctggcgcatatgatcaaatttcggggacac
ttcctcatcgagggggacctgaacccagacaacagcgatgtcgataaact
ctttatccaactggttcagacttacaatcagcttttcgaagagaacccga
tcaacgcatccggagttgacgccaaagcaatcctgagcgctaggctgtcc
aaatcccggcggctcgaaaacctcatcgcacagctccctggggagaagaa
gaacggcctgtttggtaatcttatcgccctgtcactcgggctgaccccca
actttaaatctaacttcgacctggccgaagatgccaagcttcaactgagc
aaagacacctacgatgatgatctcgacaatctgctggcccagatcggcga
ccagtacgcagacctttttttggcggcaaagaacctgtcagacgccattc
tgctgagtgatattctgcgagtgaacacggagatcaccaaagctccgctg
agcgctagtatgatcaagcgctatgatgagcaccaccaagacttgacttt
gctgaaggcccttgtcagacagcaactgcctgagaagtacaaggaaattt
tcttcgatcagtctaaaaatggctacgccggatacattgacggcggagca
agccaggaggaattttacaaatttattaagcccatcttggaaaaaatgga
cggcaccgaggagctgctggtaaagcttaacagagaagatctgttgcgca
aacagcgcactttcgacaatggaagcatcccccaccagattcacctgggc
gaactgcacgctatcctcaggcggcaagaggatttctacccctttttgaa
agataacagggaaaagattgagaaaatcctcacatttcggataccctact
atgtaggccccctcgcccggggaaattccagattcgcgtggatgactcgc
aaatcagaagagaccatcactccctggaacttcgaggaagtcgtggataa
gggggcctctgcccagtccttcatcgaaaggatgactaactttgataaaa
atctgcctaacgaaaaggtgcttcctaaacactctctgctgtacgagtac
ttcacagtttataacgagctcaccaaggtcaaatacgtcacagaagggat
gagaaagccagcattcctgtctggagagcagaagaaagctatcgtggacc
tcctcttcaagacgaaccggaaagttaccgtgaaacagctcaaagaagac
tatttcaaaaagattgaatgtttcgactctgttgaaatcagcggagtgga
ggatcgcttcaacgcatccctgggaacgtatcacgatctcctgaaaatca
ttaaagacaaggacttcctggacaatgaggagaacgaggacattcttgag
gacattgtcctcacccttacgttgtttgaagatagggagatgattgaaga
acgcttgaaaacttacgctcatctcttcgacgacaaagtcatgaaacagc
tcaagaggcgccgatatacaggatgggggcggctgtcaagaaaactgatc
aatgggatccgagacaagcagagtggaaagacaatcctggattttcttaa
gtccgatggatttgccaaccggaacttcatgcagttgatccatgatgact
ctctcacctttaaggaggacatccagaaagcacaagtttctggccagggg
gacagtcttcacgagcacatcgctaatcttgcaggtagcccagctatcaa
aaagggaatactgcagaccgttaaggtcgtggatgaactcgtcaaagtaa
tgggaaggcataagcccgagaatatcgttatcgagatggcccgagagaac
caaactacccagaagggacagaagaacagtagggaaaggatgaagaggat
tgaagagggtataaaagaactggggtcccaaatccttaaggaacacccag
ttgaaaacacccagcttcagaatgagaagctctacctgtactacctgcag
aacggcagggacatgtacgtggatcaggaactggacatcaatcggctctc
cgactacgacgtggctgctatcgtgccccagtcttttctcaaagatgatt
ctattgataataaagtgttgacaagatccgataaagctagagggaagagt
gataacgtcccctcagaagaagttgtcaagaaaatgaaaaattattggcg
gcagctgctgaacgccaaactgatcacacaacggaagttcgataatctga
ctaaggctgaacgaggtggcctgtctgagttggataaagccggcttcatc
aaaaggcagcttgttgagacacgccagatcaccaagcacgtggcccaaat
tctcgattcacgcatgaacaccaagtacgatgaaaatgacaaactgattc
gagaggtgaaagttattactctgaagtctaagctggtctcagatttcaga
aaggactttcagttttataaggtgagagagatcaacaattaccaccatgc
gcatgatgcctacctgaatgcagtggtaggcactgcacttatcaaaaaat
atcccaagcttgaatctgaatttgtttacggagactataaagtgtacgat
gttaggaaaatgatcgcaaagtctgagcaggaaataggcaaggccaccgc
taagtacttcttttacagcaatattatgaattttttcaagaccgagatta
cactggccaatggagagattcggaagcgaccacttatcgaaacaaacgga
gaaacaggagaaatcgtgtgggacaagggtagggatttcgcgacagtccg
gaaggtcctgtccatgccgcaggtgaacatcgttaaaaagaccgaagtac
agaccggaggcttctccaaggaaagtatcctcccgaaaaggaacagcgac
aagctgatcgcacgcaaaaaagattgggaccccaagaaatacggcggatt
cgattctcctacagtcgcttacagtgtactggttgtggccaaagtggaga
aagggaagtctaaaaaactcaaaagcgtcaaggaactgctgggcatcaca
atcatggagcgatcaagcttcgaaaaaaaccccatcgactttctcgaggc
gaaaggatataaagaggtcaaaaaagacctcatcattaagcttcccaagt
actctctctttgagcttgaaaacggccggaaacgaatgctcgctagtgcg
ggcgagctgcagaaaggtaacgagctggcactgccctctaaatacgttaa
tttcttgtatctggccagccactatgaaaagctcaaagggtctcccgaag
ataatgagcagaagcagctgttcgtggaacaacacaaacactaccttgat
gagatcatcgagcaaataagcgaattctccaaaagagtgatcctcgccga
cgctaacctcgataaggtgctttctgcttacaataagcacagggataagc
ccatcagggagcaggcagaaaacattatccacttgtttactctgaccaac
ttgggcgcgcctgcagccttcaagtacttcgacaccaccatagacagaaa
gcggtacacctctacaaaggaggtcctggacgccacactgattcatcagt
caattacggggctctatgaaacaagaatcgacctctctcagctcggtgga
gacagcagggctgaccccaagaagaagaggaaggtgtcgccagggatccg
tcgacttgacgcgttgatatcaacaagtttgtacaaaaaagcaggctaca
aagaggccagcggttccggacgggctgacgcattggacgattttgatctg
gatatgctgggaagtgacgccctcgatgattttgaccttgacatgcttgg
ttcggatgcccttgatgactttgacctcgacatgctcggcagtgacgccc
ttgatgatttcgacctggacatgctgattaactctagaagttccggatct
ccgaaaaagaaacgcaaagttggtagccagtacctgcccgacaccgacga
ccggcaccggatcgaggaaaagcggaagcggacctacgagacattcaaga
gcatcatgaagaagtcccccttcagcggccccaccgaccctagacctcca
cctagaagaatcgccgtgcccagcagatccagcgccagcgtgccaaaacc
tgccccccagccttaccccttcaccagcagcctgagcaccatcaactacg
acgagttccctaccatggtgttccccagcggccagatctctcaggcctct
gctctggctccagcccctcctcaggtgctgcctcaggctcctgctcctgc
accagctccagccatggtgtctgcactggctcaggcaccagcacccgtgc
ctgtgctggctcctggacctccacaggctgtggctccaccagcccctaaa
cctacacaggccggcgagggcacactgtctgaagctctgctgcagctgca
gttcgacgacgaggatctgggagccctgctgggaaacagcaccgatcctg
ccgtgttcaccgacctggccagcgtggacaacagcgagttccagcagctg
ctgaaccagggcatccctgtggcccctcacaccaccgagcccatgctgat
ggaataccccgaggccatcacccggctcgtgacaggcgctcagaggcctc
ctgatccagctcctgcccctctgggagcaccaggcctgcctaatggactg
ctgtctggcgacgaggacttcagctctatcgccgatatggatttctcagc
cttgctgggctctggcagcggcagccgggattccagggaagggatgtttt
tgccgaagcctgaggccggctccgctattagtgacgtgtttgagggccgc
gaggtgtgccagccaaaacgaatccggccatttcatcctccaggaagtcc
atgggccaaccgcccactccccgccagcctcgcaccaacaccaaccggtc
cagtacatgagccagtcgggtcactgaccccggcaccagtccctcagcca
ctggatccagcgcccgcagtgactcccgaggccagtcacctgttggagga
tcccgatgaagagacgagccaggctgtcaaagcccttcgggagatggccg
atactgtgattccccagaaggaagaggctgcaatctgtggccaaatggac
ctttcccatccgcccccaaggggccatctggatgagctgacaaccacact
tgagtccatgaccgaggatctgaacctggactcacccctgaccccggaat
tgaacgagattctggataccttcctgaacgacgagtgcctcttgcatgcc
atgcatatcagcacaggactgtccatcttcgacacatctctgttttg

1. A genetic circuit for the regulated expression of a target gene, the circuit comprising an inducible nucleolytically-inactive CRISPR-associated protein comprising an activation or repression domain operably linked at the N-terminus of the protein and a sequence specific guide RNA, where the gRNA regulates the expression of the target gene.

2. The genetic circuit of claim 1, wherein the CRISPR-associated protein is dCas9 or dCpf1.

3. The genetic circuit of claim 1, wherein the circuit regulates expression of an endogenous gene in response to stimuli, thereby emulating a logic gate.

4. The genetic circuit of claim 3, wherein the endogenous gene encodes a BMP.

5. The genetic circuit of claim 1, wherein the activation/repression domain is VPR, SAM, or KRAB.

6. The genetic circuit of claim 1, wherein the target gene encodes an endogenous or recombinant protein.

7. A cell comprising the genetic circuit of claim 1.

8. The cell of claim 5, wherein the cell is a fibroblast, adipose-derived stem cell, myocyte, cartilaginous cell, or osteoblast.

9. A system for regulating a biological process, the system comprising
a cell comprising a nucleolytically-inactive CRISPR-associated protein comprising an inducible activation or repression domain operably linked at the N-terminus of the protein and a sequence specific guide RNA, where the gRNA regulates the expression of the target gene; and
a substrate comprising an agent capable of inducing the activation or repression domain.

10. The system of claim 9, wherein the substrate is an electrospun template.

11. The system of claim 9, wherein the induction is by a small molecule drug, light, cytokine, antibiotic, pH, oxygen level, superoxide, cumate or doxycycline.

12. The system of claim 9, wherein the cell is genetically modified to express a blue light-inducible sleeping beauty transposon.

13. The system of claim 9, wherein the substrate is modified to generate in a cell in contact with the substrate a gradient of growth factors.

14. A method for regenerating a tissue, the method comprising contacting a cell comprising a nucleolytically-inactive CRISPR-associated protein comprising an inducible activation or repression domain operably linked at the N-terminus of the protein and a sequence specific guide RNA, where the gRNA regulates the expression of the target gene; and a substrate comprising an agent capable of inducing the activation or repression domain.

15. The method of claim 14, wherein the target gene is BMP-2 or PDGF-BB.

16. The method of claim 14, wherein the biological process is tissue regeneration.

17. The method of claim 16, wherein the method generates an enthesis.
